SEQUENCE LISTING (137 sequences)

SEQ ID NO: 1 - protein, 475 aa, Artificial sequence
  synthetic sequence
MGWSLILLFLVAVATRVHSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGHHHHHH

SEQ ID NO: 2 - protein, 450 aa, Artificial sequence
  synthetic sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGG

SEQ ID NO: 3 - protein, 234 aa, Artificial sequence
  synthetic sequence
MRVPAQLLGLLLLWLPGARCDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 214 aa, Artificial sequence
  synthetic sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 476 aa, Artificial sequence
  synthetic sequence
MGWSCIILFLVATATGVHSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGHHHHHH

SEQ ID NO: 6 - protein, 451 aa, Artificial sequence
  synthetic sequence
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGG

SEQ ID NO: 7 - protein, 232 aa, Artificial sequence
  synthetic sequence
MGWSCIILFLVATATGVHSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 213 aa, Artificial sequence
  synthetic sequence
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 474 aa, Artificial sequence
  synthetic sequence
MGWSCIILFLVATATGVHSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGHHHHHH

SEQ ID NO: 10 - protein, 449 aa, Artificial sequence
  synthetic sequence
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGG

SEQ ID NO: 11 - protein, 233 aa, Artificial sequence
  synthetic sequence
MGWSCIILFLVATATGVHSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 214 aa, Artificial sequence
  synthetic sequence
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 595 aa, Artificial sequence
  synthetic sequence
MLLLLLLPLLWGRERVEGQKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGGGGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 14 - protein, 568 aa, Artificial sequence
  synthetic sequence
QKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGGGGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGG

SEQ ID NO: 15 - protein, 331 aa, Artificial sequence
  synthetic sequence
QKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVS

SEQ ID NO: 16 - protein, 594 aa, Artificial sequence
  synthetic sequence
MLLLLLLPLLWGRERVEGQKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFAMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGGGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 17 - protein, 567 aa, Artificial sequence
  synthetic sequence
QKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFAMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGGGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGG

SEQ ID NO: 18 - protein, 331 aa, Artificial sequence
  synthetic sequence
QKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFAMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVS

SEQ ID NO: 19 - protein, 588 aa, Artificial sequence
  synthetic sequence
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGGGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 20 - protein, 571 aa, Artificial sequence
  synthetic sequence
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGGGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 21 - protein, 326 aa, Artificial sequence
  synthetic sequence
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATS

SEQ ID NO: 22 - protein, 588 aa, Artificial sequence
  synthetic sequence
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFAMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGGGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 23 - protein, 562 aa, Artificial sequence
  synthetic sequence
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFAMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGGGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGG

SEQ ID NO: 24 - protein, 326 aa, Artificial sequence
  synthetic sequence
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFAMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATS

SEQ ID NO: 25 - protein, 595 aa, Artificial sequence
  synthetic sequence
MLLLLLLPLLWGRERVEGQKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFAMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGGGGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 26 - protein, 791 aa, Artificial sequence
  synthetic sequence
MLLPLLLSSLLGGSQAMDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLISTGGGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 27 - protein, 766 aa, Artificial sequence
  synthetic sequence
MDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLISTGGGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGG

SEQ ID NO: 28 - protein, 530 aa, Artificial sequence
  synthetic sequence
MDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLIST

SEQ ID NO: 29 - protein, 475 aa, Artificial sequence
  synthetic sequence
MGWSCIILFLVATATGVHSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGHHHHHH

SEQ ID NO: 30 - protein, 450 aa, Artificial sequence
  synthetic sequence
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGG

SEQ ID NO: 31 - protein, 237 aa, Artificial sequence
  synthetic sequence
MGWSCIILFLVATATGVHSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 218 aa, Artificial sequence
  synthetic sequence
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 468 aa, Artificial sequence
  synthetic sequence
MGWSCIILFLVATATGVHSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGHHHHHH

SEQ ID NO: 34 - protein, 443 aa, Artificial sequence
  synthetic sequence
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGG

SEQ ID NO: 35 - protein, 233 aa, Artificial sequence
  synthetic sequence
MGWSCIILFLVATATGVHSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 214 aa, Artificial sequence
  synthetic sequence
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 595 aa, Artificial sequence
  synthetic sequence
MLLLLLLPLLWGRERVEGQKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 38 - protein, 568 aa, Artificial sequence
  synthetic sequence
QKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGG

SEQ ID NO: 39 - protein, 588 aa, Artificial sequence
  synthetic sequence
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 40 - protein, 562 aa, Artificial sequence
  synthetic sequence
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGG

SEQ ID NO: 41 - protein, 584 aa, Artificial sequence
  synthetic sequence
MLLPLLLSSLLGGSQAMDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSGGGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 42 - protein, 559 aa, Artificial sequence
  synthetic sequence
MDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSGGGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGG

SEQ ID NO: 43 - protein, 323 aa, Artificial sequence
  synthetic sequence
MDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLS

SEQ ID NO: 44 - protein, 393 aa, Artificial sequence
  synthetic sequence
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 45 - protein, 368 aa, Artificial sequence
  synthetic sequence
FSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGG

SEQ ID NO: 46 - protein, 132 aa, Artificial sequence
  synthetic sequence
FSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQ

SEQ ID NO: 47 - protein, 476 aa, Artificial sequence
  synthetic sequence
MGWSCIILFLVATATGVHSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGHHHHHH

SEQ ID NO: 48 - protein, 451 aa, Artificial sequence
  synthetic sequence
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGG

SEQ ID NO: 49 - protein, 476 aa, Artificial sequence
  synthetic sequence
MGWSCIILFLVATATGVHSEVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGHHHHHH

SEQ ID NO: 50 - protein, 451 aa, Artificial sequence
  synthetic sequence
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGG

SEQ ID NO: 51 - protein, 238 aa, Artificial sequence
  synthetic sequence
MGWSCIILFLVATATGVHSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 219 aa, Artificial sequence
  synthetic sequence
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 473 aa, Artificial sequence
  synthetic sequence
MGWSCIILFLVATATGVHSEVQLVESGGGLVQPGGSLRLSCAASGFTFSGYGMSWVRQAPGKGLEWVATITSGGTYTYYPDSVKGRFTISRDNAKNSLYLQMNSLRAEDTAVYYCARSLAGNAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGHHHHHH

SEQ ID NO: 54 - protein, 448 aa, Artificial sequence
  synthetic sequence
EVQLVESGGGLVQPGGSLRLSCAASGFTFSGYGMSWVRQAPGKGLEWVATITSGGTYTYYPDSVKGRFTISRDNAKNSLYLQMNSLRAEDTAVYYCARSLAGNAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGG

SEQ ID NO: 55 - protein, 233 aa, Artificial sequence
  synthetic sequence
MGWSCIILFLVATATGVHSEIVLTQSPATLSLSPGERATLSCRASQTISDYLHWYQQKPGQAPRLLIKFASQSISGIPARFSGSGSGTDFTLTISSLEPEDFAVYYCQNGHGFPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 214 aa, Artificial sequence
  synthetic sequence
EIVLTQSPATLSLSPGERATLSCRASQTISDYLHWYQQKPGQAPRLLIKFASQSISGIPARFSGSGSGTDFTLTISSLEPEDFAVYYCQNGHGFPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 472 aa, Artificial sequence
  synthetic sequence
MGWSCIILFLVATATGVHSQVQLQQPGAELVKPGASVMMSCKASGYTFTNYNMHWVKQTPGQGLEWIGTIYPGNDDTSYNQKFKDKATLTADKSSSAAYMQLSSLTSEDSAVYYCARGGYRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGHHHHHH

SEQ ID NO: 58 - protein, 447 aa, Artificial sequence
  synthetic sequence
QVQLQQPGAELVKPGASVMMSCKASGYTFTNYNMHWVKQTPGQGLEWIGTIYPGNDDTSYNQKFKDKATLTADKSSSAAYMQLSSLTSEDSAVYYCARGGYRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGG

SEQ ID NO: 59 - protein, 238 aa, Artificial sequence
  synthetic sequence
MGWSCIILFLVATATGVHSDVLMTQTPLSLPVSLGDQASISCRSSQSIVYSNGNTYLGWYLQKPGQSPKLLIYKVSNRFSGVPDRFSGSGSGTDFTLKISRVEAEDLGVYHCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 219 aa, Artificial sequence
  synthetic sequence
DVLMTQTPLSLPVSLGDQASISCRSSQSIVYSNGNTYLGWYLQKPGQSPKLLIYKVSNRFSGVPDRFSGSGSGTDFTLKISRVEAEDLGVYHCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 475 aa, Artificial sequence
  synthetic sequence
MGWSCIILFLVATATGVHSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGHHHHHH

SEQ ID NO: 62 - protein, 450 aa, Artificial sequence
  synthetic sequence
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGG

SEQ ID NO: 63 - protein, 234 aa, Artificial sequence
  synthetic sequence
MGWSCIILFLVATATGVHSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 64 - protein, 215 aa, Artificial sequence
  synthetic sequence
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 65 - protein, 473 aa, Artificial sequence
  synthetic sequence
MGWSCIILFLVATATGVHSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGHHHHHH

SEQ ID NO: 66 - protein, 448 aa, Artificial sequence
  synthetic sequence
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGG

SEQ ID NO: 67 - protein, 233 aa, Artificial sequence
  synthetic sequence
MGWSCIILFLVATATGVHSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 214 aa, Artificial sequence
  synthetic sequence
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 932 aa, Artificial sequence
  synthetic sequence
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACARCNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 70 - protein, 904 aa, Artificial sequence
  synthetic sequence
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACARCNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGG

SEQ ID NO: 71 - protein, 668 aa, Artificial sequence
  synthetic sequence
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACARCNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 72 - protein, 571 aa, Artificial sequence
  synthetic sequence
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 73 - protein, 543 aa, Artificial sequence
  synthetic sequence
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGG

SEQ ID NO: 74 - protein, 307 aa, Artificial sequence
  synthetic sequence
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQ

SEQ ID NO: 75 - protein, 584 aa, Artificial sequence
  synthetic sequence
MLLPLLLSSLLGGSQAMDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 76 - protein, 559 aa, Artificial sequence
  synthetic sequence
MDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGG

SEQ ID NO: 77 - protein, 546 aa, Artificial sequence
  synthetic sequence
MIASQFLSALTLVLLIKESGAWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 78 - protein, 516 aa, Artificial sequence
  synthetic sequence
WSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGG

SEQ ID NO: 79 - protein, 280 aa, Artificial sequence
  synthetic sequence
WSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKA

SEQ ID NO: 80 - protein, 566 aa, Artificial sequence
  synthetic sequence
MANCQIAILYQRFQRVVFGISQLLCFSALISELTNQKEVAAWTYHYSTKAYSWNISRKYCQNRYTDLVAIQNKNEIDYLNKVLPYYSSYYWIGIRKNNKTWTWVGTKKALTNEAENWADNEPNNKRNNEDCVEIYIKSPSAPGKWNDEHCLKKKHALCYTASCQDMSCSKQGECLETIGNYTCSCYPGFYGPECEYVRECGELELPQHVLMNCSHPLGNFSFNSQCSFHCTDGYQVNGPSKLECLASGIWTNKPPQCLAAQCPPLKIPERGNMTCLHSAKAFQHQSSCSFSCEEGFALVGPEVVQCTASGVWTAPAPVCKAGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 81 - protein, 516 aa, Artificial sequence
  synthetic sequence
WTYHYSTKAYSWNISRKYCQNRYTDLVAIQNKNEIDYLNKVLPYYSSYYWIGIRKNNKTWTWVGTKKALTNEAENWADNEPNNKRNNEDCVEIYIKSPSAPGKWNDEHCLKKKHALCYTASCQDMSCSKQGECLETIGNYTCSCYPGFYGPECEYVRECGELELPQHVLMNCSHPLGNFSFNSQCSFHCTDGYQVNGPSKLECLASGIWTNKPPQCLAAQCPPLKIPERGNMTCLHSAKAFQHQSSCSFSCEEGFALVGPEVVQCTASGVWTAPAPVCKAGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGG

SEQ ID NO: 82 - protein, 280 aa, Artificial sequence
  synthetic sequence
WTYHYSTKAYSWNISRKYCQNRYTDLVAIQNKNEIDYLNKVLPYYSSYYWIGIRKNNKTWTWVGTKKALTNEAENWADNEPNNKRNNEDCVEIYIKSPSAPGKWNDEHCLKKKHALCYTASCQDMSCSKQGECLETIGNYTCSCYPGFYGPECEYVRECGELELPQHVLMNCSHPLGNFSFNSQCSFHCTDGYQVNGPSKLECLASGIWTNKPPQCLAAQCPPLKIPERGNMTCLHSAKAFQHQSSCSFSCEEGFALVGPEVVQCTASGVWTAPAPVCKA

SEQ ID NO: 83 - protein, 474 aa, Artificial sequence
  synthetic sequence
MGWSCIILFLVATATGVHSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGHHHHHH

SEQ ID NO: 84 - protein, 449 aa, Artificial sequence
  synthetic sequence
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGG

SEQ ID NO: 85 - protein, 232 aa, Artificial sequence
  synthetic sequence
MGWSCIILFLVATATGVHSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 213 aa, Artificial sequence
  synthetic sequence
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 476 aa, Artificial sequence
  synthetic sequence
MGWSCIILFLVATATGVHSEVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGHHHHHH

SEQ ID NO: 88 - protein, 451 aa, Artificial sequence
  synthetic sequence
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGG

SEQ ID NO: 89 - protein, 234 aa, Artificial sequence
  synthetic sequence
MGWSCIILFLVATATGVHSEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 215 aa, Artificial sequence
  synthetic sequence
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 396 aa, Artificial sequence
  synthetic sequence
MLLLLLPLLWGRERAEGQACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTKLPDGYEFKFPNRLNLEAINYMAADGDFKIKCVAFDGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 92 - protein, 370 aa, Artificial sequence
  synthetic sequence
QACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTKLPDGYEFKFPNRLNLEAINYMAADGDFKIKCVAFDGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGG

SEQ ID NO: 93 - protein, 134 aa, Artificial sequence
  synthetic sequence
QACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTKLPDGYEFKFPNRLNLEAINYMAADGDFKIKCVAFD

SEQ ID NO: 94 - protein, 512 aa, Artificial sequence
  synthetic sequence
MLLLLLPLLWGRERAEGQADNFSLHDALSGSGNPNPQGWPGAWGNQPAGAGGYPGASYPGAYPGQAPPGAYPGQAPPGAYHGAPGAYPGAPAPGVYPGPPSGPGAYPSSGQPSAPGAYPATGPYGAPAGPLIVPYNLPLPGGVVPRMLITILGTVKPNANRIALDFQRGNDVAFHFNPRFNENNRRVIVCNTKLDNNWGREERQSVFPFESGKPFKIHVLVEPDHFKVAVNDAHLLQYNHRVKKLNEISKLGISGDIDLTSASYTMIGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGYPYDVPDYA

SEQ ID NO: 95 - protein, 486 aa, Artificial sequence
  synthetic sequence
QADNFSLHDALSGSGNPNPQGWPGAWGNQPAGAGGYPGASYPGAYPGQAPPGAYPGQAPPGAYHGAPGAYPGAPAPGVYPGPPSGPGAYPSSGQPSAPGAYPATGPYGAPAGPLIVPYNLPLPGGVVPRMLITILGTVKPNANRIALDFQRGNDVAFHFNPRFNENNRRVIVCNTKLDNNWGREERQSVFPFESGKPFKIHVLVEPDHFKVAVNDAHLLQYNHRVKKLNEISKLGISGDIDLTSASYTMIGGGGGPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGG

SEQ ID NO: 96 - protein, 250 aa, Artificial sequence
  synthetic sequence
QADNFSLHDALSGSGNPNPQGWPGAWGNQPAGAGGYPGASYPGAYPGQAPPGAYPGQAPPGAYHGAPGAYPGAPAPGVYPGPPSGPGAYPSSGQPSAPGAYPATGPYGAPAGPLIVPYNLPLPGGVVPRMLITILGTVKPNANRIALDFQRGNDVAFHFNPRFNENNRRVIVCNTKLDNNWGREERQSVFPFESGKPFKIHVLVEPDHFKVAVNDAHLLQYNHRVKKLNEISKLGISGDIDLTSASYTMI

SEQ ID NO: 97 - DNA, 1428 bp, Artificial sequence
  synthetic sequence
atgggttggagcctcatcttgctcttccttgtcgctgttgctacgcgtgtccactccgaagtgcagctggtggagtctggcggaggactggtgcagccagggggcagcctgagactgtcttgcgccgcctccggcttcaacatcaaggacacctacatccactgggtccgccaggcaccaggcaagggactggaatgggtggcccggatctaccctaccaacggctacaccagatacgccgactccgtgaagggccggttcaccatctccgccgacacctccaagaacaccgcctacctgcagatgaattccctgagggccgaggacaccgccgtgtactactgctccagatggggaggcgacggcttctacgccatggactactggggccagggcaccctggtcacagtgtcctctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgggcatcaccaccatcaccattga

SEQ ID NO: 98 - DNA, 705 bp, Artificial sequence
  synthetic sequence
atgagggtccccgctcagctcctggggctcctgctgctctggctcccaggtgcacgatgtgacatccagatgacccagtccccctcctccctgtctgcctccgtgggcgacagagtgaccatcacctgtcgggcctcccaggatgtgaacaccgccgtggcctggtatcagcagaagcctggcaaggcccctaagctgctgatctactccgcctccttcctgtactccggcgtgccctcccggttctccggctccagatccggcaccgacttcaccctgaccatctccagcctgcagcctgaggacttcgccacctactactgccagcagcactacaccacccctccaaccttcggccagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 99 - DNA, 1431 bp, Artificial sequence
  synthetic sequence
atgggatggtcatgtatcatcctttttctagtagcaactgcaaccggtgtacattcccaggtacaactgcagcagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgcagacaaatcctccagcacagcctacatgcagctcagcagcctgacatctgaggactctgcggtctattactgtgcaagatcgacttactacggcggtgactggtacttcaatgtctggggcgcagggaccacggtcaccgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgggcatcaccaccatcaccattga

SEQ ID NO: 100 - DNA, 699 bp, Artificial sequence
  synthetic sequence
atgggatggtcatgtatcatcctttttctagtagcaactgcaaccggtgtacattcacaaattgttctctcccagtctccagcaatcctgtctgcatctccaggggagaaggtcacaatgacttgcagggccagctcaagtgtaagttacatccactggttccagcagaagccaggatcctcccccaaaccctggatttatgccacatccaacctggcttctggagtccctgttcgcttcagtggcagtgggtctgggacttcttactctctcacaatcagcagagtggaggctgaagatgctgccacttattactgccagcagtggactagtaacccacccacgttcggaggggggaccaagctggaaatcaaacgtacggtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatgctga

SEQ ID NO: 101 - DNA, 1425 bp, Artificial sequence
  synthetic sequence
atgggatggtcatgtatcatcctttttctagtagcaactgcaaccggtgtacattcccaggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcagagcaacgataccgcgatttattattgcgcgcgcgcgctgacctattatgattatgaatttgcgtattggggccagggcaccctggtgaccgtgagcgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgggcatcaccaccatcaccattga

SEQ ID NO: 102 - DNA, 702 bp, Artificial sequence
  synthetic sequence
atgggatggtcatgtatcatcctttttctagtagcaactgcaaccggtgtacattccgacatcctgctcacccagagcccggtgatcctgtcggtcagccccggcgagcgggtgagcttcagctgccgcgccagccagtcgatcgggacgaacatccactggtaccagcagcggaccaacggcagcccccgcctgctcatcaagtacgcgagcgagagcatcagcgggatcccctcgcggttcagcggcagcgggagcggcaccgacttcaccctgagcatcaacagcgtggagtcggaggacatcgccgactactactgccagcagaacaacaactggccgacgaccttcggcgccgggaccaagctggagctcaagcgcaccgtcgccgcgcccagcgtgttcatcttcccgcccagcgacgagcagctgaagagcggcacggccagcgtggtctgcctgctcaacaacttctacccccgggaggccaaggtgcagtggaaggtggacaacgccctgcagtcggggaacagccaggagagcgtcaccgagcaggacagcaaggacagcacctacagcctgtcgagcaccctcacgctgagcaaggccgactacgagaagcacaaggtgtacgcgtgcgaggtgacccaccagggcctgagcagccccgtcaccaagtcgttcaaccgcggagaatgctga

SEQ ID NO: 103 - DNA, 1788 bp, Artificial sequence
  synthetic sequence
atgctgctgctgctgctgctgcccctgctctgggggagggagagggtggaaggacagaagagtaaccggaaggattactcgctgacgatgcagagttccgtgaccgtgcaagagggcatgtgtgtccatgtgcgctgctccttctcctacccagtggacagccagactgactctgacccagttcatggctactggttccgggcagggaatgatataagctggaaggctccagtggccacaaacaacccagcttgggcagtgcaggaggaaactcgggaccgattccacctccttggggacccacagaccaaaaattgcaccctgagcatcagagatgccagaatgagtgatgcggggagatacttctttcgtatggagaaaggaaatataaaatggaattataaatatgaccagctctctgtgaacgtgacagccttgacccacaggcccaacatccttatccccggtaccctggagtctggctgcttccagaatctgacctgctctgtgccctgggcctgtgagcaggggacgccccctatgatctcctggatggggacctctgtgtcccccctgcacccctccaccacccgctcctcagtgctcaccctcatcccacagccccagcaccacggcaccagcctcacctgtcaggtgaccttgcctggggccggcgtgaccacgaacaggaccatccaactcaatgtgtcctaccctcctcagaacttgactgtgactgtcttccaaggagaaggcacagcatccacagctctggggaacagctcatctctttcagtcctagagggccagtctctgcgcttggtctgtgctgttgacagcaatccccctgccaggctgagctggacctggaggagtctgaccctgtacccctcacagccctcaaaccctctggtactggagctgcaagtgcacctgggggatgaaggggaattcacctgtcgagctcagaactctctgggttcccagcacgtttccctgaacctctccctgcaacaggagtacacaggcaaaatgaggcctgtatcaggaggcggaggtggaggccccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtgggtacccatacgatgttccagattacgcttga

SEQ ID NO: 104 - DNA, 1788 bp, Artificial sequence
  synthetic sequence
atgctgctgctgctgctgctgcccctgctctgggggagggagagggtggaaggacagaagagtaaccggaaggattactcgctgacgatgcagagttccgtgaccgtgcaagagggcatgtgtgtccatgtgcgctgctccttctcctacccagtggacagccagactgactctgacccagttcatggctactggttccgggcagggaatgatataagctggaaggctccagtggccacaaacaacccagcttgggcagtgcaggaggaaactcgggaccgattccacctccttggggacccacagaccaaaaattgcaccctgagcatcagagatgccagaatgagtgatgcggggagatacttctttgccatggagaaaggaaatataaaatggaattataaatatgaccagctctctgtgaacgtgacagccttgacccacaggcccaacatccttatccccggtaccctggagtctggctgcttccagaatctgacctgctctgtgccctgggcctgtgagcaggggacgccccctatgatctcctggatggggacctctgtgtcccccctgcacccctccaccacccgctcctcagtgctcaccctcatcccacagccccagcaccacggcaccagcctcacctgtcaggtgaccttgcctggggccggcgtgaccacgaacaggaccatccaactcaatgtgtcctaccctcctcagaacttgactgtgactgtcttccaaggagaaggcacagcatccacagctctggggaacagctcatctctttcagtcctagagggccagtctctgcgcttggtctgtgctgttgacagcaatccccctgccaggctgagctggacctggaggagtctgaccctgtacccctcacagccctcaaaccctctggtactggagctgcaagtgcacctgggggatgaaggggaattcacctgtcgagctcagaactctctgggttcccagcacgtttccctgaacctctccctgcaacaggagtacacaggcaaaatgaggcctgtatcaggaggcggaggtggaggccccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtgggtacccatacgatgttccagattacgcttga

SEQ ID NO: 105 - DNA, 1767 bp, Artificial sequence
  synthetic sequence
atgctgctgctgctgctgcccctgctctgggggagggagagggcggaaggacagacaagtaaactgctgacgatgcagagttccgtgacggtgcaggaaggcctgtgtgtccatgtgccctgctccttctcctacccctcgcatggctggatttaccctggcccagtagttcatggctactggttccgggaaggggccaatacagaccaggatgctccagtggccacaaacaacccagctcgggcagtgtgggaggagactcgggaccgattccacctccttggggacccacataccaagaattgcaccctgagcatcagagatgccagaagaagtgatgcggggagatacttctttcgtatggagaaaggaagtataaaatggaattataaacatcaccggctctctgtgaatgtgacagccttgacccacaggcccaacatcctcatcccaggcaccctggagtccggctgcccccagaatctgacctgctctgtgccctgggcctgtgagcaggggacaccccctatgatctcctggatagggacctccgtgtcccccctggacccctccaccacccgctcctcggtgctcaccctcatcccacagccccaggaccatggcaccagcctcacctgtcaggtgaccttccctggggccagcgtgaccacgaacaagaccgtccatctcaacgtgtcctacccgcctcagaacttgaccatgactgtcttccaaggagacggcacagtatccacagtcttgggaaatggctcatctctgtcactcccagagggccagtctctgcgcctggtctgtgcagttgatgcagttgacagcaatccccctgccaggctgagcctgagctggagaggcctgaccctgtgcccctcacagccctcaaacccgggggtgctggagctgccttgggtgcacctgagggatgcagctgaattcacctgcagagctcagaaccctctcggctctcagcaggtctacctgaacgtctccctgcagagcaaagccacatcaggcggaggtggaggccccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtgggtacccatacgatgttccagattacgcttga

SEQ ID NO: 106 - DNA, 1767 bp, Artificial sequence
  synthetic sequence
atgctgctgctgctgctgcccctgctctgggggagggagagggcggaaggacagacaagtaaactgctgacgatgcagagttccgtgacggtgcaggaaggcctgtgtgtccatgtgccctgctccttctcctacccctcgcatggctggatttaccctggcccagtagttcatggctactggttccgggaaggggccaatacagaccaggatgctccagtggccacaaacaacccagctcgggcagtgtgggaggagactcgggaccgattccacctccttggggacccacataccaagaattgcaccctgagcatcagagatgccagaagaagtgatgcggggagatacttctttgccatggagaaaggaagtataaaatggaattataaacatcaccggctctctgtgaatgtgacagccttgacccacaggcccaacatcctcatcccaggcaccctggagtccggctgcccccagaatctgacctgctctgtgccctgggcctgtgagcaggggacaccccctatgatctcctggatagggacctccgtgtcccccctggacccctccaccacccgctcctcggtgctcaccctcatcccacagccccaggaccatggcaccagcctcacctgtcaggtgaccttccctggggccagcgtgaccacgaacaagaccgtccatctcaacgtgtcctacccgcctcagaacttgaccatgactgtcttccaaggagacggcacagtatccacagtcttgggaaatggctcatctctgtcactcccagagggccagtctctgcgcctggtctgtgcagttgatgcagttgacagcaatccccctgccaggctgagcctgagctggagaggcctgaccctgtgcccctcacagccctcaaacccgggggtgctggagctgccttgggtgcacctgagggatgcagctgaattcacctgcagagctcagaaccctctcggctctcagcaggtctacctgaacgtctccctgcagagcaaagccacatcaggcggaggtggaggccccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtgggtacccatacgatgttccagattacgcttga

SEQ ID NO: 107 - DNA, 2376 bp, Artificial sequence
  synthetic sequence
atgctactgccactgctgctgtcctcgctgctgggcgggtcccaggctatggatgggagattctggatacgagtgcaggagtcagtgatggtgccggagggcctgtgcatctctgtgccctgctctttctcctacccccgacaggactggacagggtctaccccagcttatggctactggttcaaagcagtgactgagacaaccaagggtgctcctgtggccacaaaccaccagagtcgagaggtggaaatgagcacccggggccgattccagctcactggggatcccgccaaggggaactgctccttggtgatcagagacgcgcagatgcaggatgagtcacagtacttctttcgggtggagagaggaagctatgtgagatataatttcatgaacgatgggttctttctaaaagtaacagccctgactcagaagcctgatgtctacatccccgagaccctggagcccgggcagccggtgacggtcatctgtgtgtttaactgggcctttgaggaatgtccacccccttctttctcctggacgggggctgccctctcctcccaaggaaccaaaccaacgacctcccacttctcagtgctcagcttcacgcccagaccccaggaccacaacaccgacctcacctgccatgtggacttctccagaaagggtgtgagcgcacagaggaccgtccgactccgtgtggcctatgcccccagagaccttgttatcagcatttcacgtgacaacacgccagccctggagccccagccccagggaaatgtcccatacctggaagcccaaaaaggccagttcctgcggctcctctgtgctgctgacagccagccccctgccacactgagctgggtcctgcagaacagagtcctctcctcgtcccatccctggggccctagacccctggggctggagctgcccggggtgaaggctggggattcagggcgctacacctgccgagcggagaacaggcttggctcccagcagcgagccctggacctctctgtgcagtatcctccagagaacctgagagtgatggtttcccaagcaaacaggacagtcctggaaaaccttgggaacggcacgtctctcccagtactggagggccaaagcctgtgcctggtctgtgtcacacacagcagccccccagccaggctgagctggacccagaggggacaggttctgagcccctcccagccctcagaccccggggtcctggagctgcctcgggttcaagtggagcacgaaggagagttcacctgccacgctcggcacccactgggctcccagcacgtctctctcagcctctccgtgcactactccccgaagctgctgggcccctcctgctcctgggaggctgagggtctgcactgcagctgctcctcccaggccagcccggccccctctctgcgctggtggcttggggaggagctgctggaggggaacagcagccaggactccttcgaggtcacccccagctcagccgggccctgggccaacagctccctgagcctccatggagggctcagctccggcctcaggctccgctgtgaggcctggaacgtccatggggcccagagtggatccatcctgcagctgccagataagaagggactcatctcaacgggcggaggtggaggccccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtgggtacccatacgatgttccagattacgcttga

SEQ ID NO: 108 - DNA, 1428 bp, Artificial sequence
  synthetic sequence
atgggatggtcatgtatcatcctttttctagtagcaactgcaaccggtgtacattcccaagtgcaattggtgcagtccggcgttgaagttaaaaagcccggtgcgtccgtgaaggtttcctgtaaggcgagtggatacaccttcacaaactattatatgtattgggtcaggcaagctccgggacagggtttggaatggatgggaggtatcaatccatccaatggtggtacgaattttaatgagaagttcaaaaatagagtgactctgactaccgacagttctacgactacagcctacatggaattgaagtccctgcaattcgacgatacggcagtttattattgtgctaggagggattatagattcgacatgggttttgattattggggccaagggactactgtaacagtatcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgggcatcaccaccatcaccattga

SEQ ID NO: 109 - DNA, 714 bp, Artificial sequence
  synthetic sequence
atgggatggtcatgtatcatcctttttctagtagcaactgcaaccggtgtacattccgaaattgttctcactcagtctccggcaacgttgtcactcagtcctggtgagagagctacgttgtcctgccgggctagcaagggagtttcaacatcaggatattcctacctccactggtaccaacagaaaccgggtcaggctccgcgcctcctcatttacctcgcatcctacctggaatctggggtgcccgcgcggttttctgggtctggctctgggactgatttcactctcactatttcctcattggaacccgaggattttgcggtgtactattgccagcactcacgggatctgcctcttacttttggaggaggtactaaagtggaaataaaacgcaccgtcgccgcgcccagcgtgttcatcttcccgcccagcgacgagcagctgaagagcggcacggccagcgtggtctgcctgctcaacaacttctacccccgggaggccaaggtgcagtggaaggtggacaacgccctgcagtcggggaacagccaggagagcgtcaccgagcaggacagcaaggacagcacctacagcctgtcgagcaccctcacgctgagcaaggccgactacgagaagcacaaggtgtacgcgtgcgaggtgacccaccagggcctgagcagccccgtcaccaagtcgttcaaccgcggagaatgctga

SEQ ID NO: 110 - DNA, 1407 bp, Artificial sequence
  synthetic sequence
atgggatggtcatgtatcatcctttttctagtagcaactgcaaccggtgtacattcccaagtgcagctcgttgaatcaggtgggggtgtggtacaacctgggagatcactgcgactggattgcaaggcttccggtatcacgttttcaaactcaggtatgcattgggtccgacaggcaccaggaaaaggactcgagtgggtagccgtaatctggtatgacggctcaaaacgctactatgcggatagtgtaaaaggaaggttcacgatctcacgcgataactctaagaacaccctgttccttcagatgaactccctgcgagcagaagatacggcagtttattattgtgcaaccaacgatgattattggggccagggaaccttggttaccgtctcatcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgggcatcaccaccatcaccattga

SEQ ID NO: 111 - DNA, 702 bp, Artificial sequence
  synthetic sequence
atgggatggtcatgtatcatcctttttctagtagcaactgcaaccggtgtacattccgagatcgttcttactcaatctcctgcaacgctttccctcagtccaggagaacgcgcgaccctttcatgcagagcctctcagagtgtttcctcatatctcgcctggtatcagcaaaaaccagggcaagcaccccgcttgttgatttacgacgcgagcaatcgggccacaggcattccagccaggttttcaggaagcggatcaggcaccgatttcacgctgaccattagctcccttgagccagaagatttcgcagtctactattgtcagcaatcatcaaactggcctcgaaccttcggccagggtacaaaagttgaaataaagcgcaccgtcgccgcgcccagcgtgttcatcttcccgcccagcgacgagcagctgaagagcggcacggccagcgtggtctgcctgctcaacaacttctacccccgggaggccaaggtgcagtggaaggtggacaacgccctgcagtcggggaacagccaggagagcgtcaccgagcaggacagcaaggacagcacctacagcctgtcgagcaccctcacgctgagcaaggccgactacgagaagcacaaggtgtacgcgtgcgaggtgacccaccagggcctgagcagccccgtcaccaagtcgttcaaccgcggagaatgctga

SEQ ID NO: 112 - DNA, 1788 bp, Artificial sequence
  synthetic sequence
atgctgctgctgctgctgctgcccctgctctgggggagggagagggtggaaggacagaagagtaaccggaaggattactcgctgacgatgcagagttccgtgaccgtgcaagagggcatgtgtgtccatgtgcgctgctccttctcctacccagtggacagccagactgactctgacccagttcatggctactggttccgggcagggaatgatataagctggaaggctccagtggccacaaacaacccagcttgggcagtgcaggaggaaactcgggaccgattccacctccttggggacccacagaccaaaaattgcaccctgagcatcagagatgccagaatgagtgatgcggggagatacttctttcgtatggagaaaggaaatataaaatggaattataaatatgaccagctctctgtgaacgtgacagccttgacccacaggcccaacatccttatccccggtaccctggagtctggctgcttccagaatctgacctgctctgtgccctgggcctgtgagcaggggacgccccctatgatctcctggatggggacctctgtgtcccccctgcacccctccaccacccgctcctcagtgctcaccctcatcccacagccccagcaccacggcaccagcctcacctgtcaggtgaccttgcctggggccggcgtgaccacgaacaggaccatccaactcaatgtgtcctaccctcctcagaacttgactgtgactgtcttccaaggagaaggcacagcatccacagctctggggaacagctcatctctttcagtcctagagggccagtctctgcgcttggtctgtgctgttgacagcaatccccctgccaggctgagctggacctggaggagtctgaccctgtacccctcacagccctcaaaccctctggtactggagctgcaagtgcacctgggggatgaaggggaattcacctgtcgagctcagaactctctgggttcccagcacgtttccctgaacctctccctgcaacaggagtacacaggcaaaatgaggcctgtatcaggaggcggaggtggaggccccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtgcaccctgcccccatcccgggatgagctgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtgggtacccatacgatgttccagattacgcttga

SEQ ID NO: 113 - DNA, 1767 bp, Artificial sequence
  synthetic sequence
atgctgctgctgctgctgcccctgctctgggggagggagagggcggaaggacagacaagtaaactgctgacgatgcagagttccgtgacggtgcaggaaggcctgtgtgtccatgtgccctgctccttctcctacccctcgcatggctggatttaccctggcccagtagttcatggctactggttccgggaaggggccaatacagaccaggatgctccagtggccacaaacaacccagctcgggcagtgtgggaggagactcgggaccgattccacctccttggggacccacataccaagaattgcaccctgagcatcagagatgccagaagaagtgatgcggggagatacttctttcgtatggagaaaggaagtataaaatggaattataaacatcaccggctctctgtgaatgtgacagccttgacccacaggcccaacatcctcatcccaggcaccctggagtccggctgcccccagaatctgacctgctctgtgccctgggcctgtgagcaggggacaccccctatgatctcctggatagggacctccgtgtcccccctggacccctccaccacccgctcctcggtgctcaccctcatcccacagccccaggaccatggcaccagcctcacctgtcaggtgaccttccctggggccagcgtgaccacgaacaagaccgtccatctcaacgtgtcctacccgcctcagaacttgaccatgactgtcttccaaggagacggcacagtatccacagtcttgggaaatggctcatctctgtcactcccagagggccagtctctgcgcctggtctgtgcagttgatgcagttgacagcaatccccctgccaggctgagcctgagctggagaggcctgaccctgtgcccctcacagccctcaaacccgggggtgctggagctgccttgggtgcacctgagggatgcagctgaattcacctgcagagctcagaaccctctcggctctcagcaggtctacctgaacgtctccctgcagagcaaagccacatcaggcggaggtggaggccccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtgcaccctgcccccatcccgggatgagctgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtgggtacccatacgatgttccagattacgcttga

SEQ ID NO: 114 - DNA, 1755 bp, Artificial sequence
  synthetic sequence
atgctactgccactgctgctgtcctcgctgctgggcgggtcccaggctatggatgggagattctggatacgagtgcaggagtcagtgatggtgccggagggcctgtgcatctctgtgccctgctctttctcctacccccgacaggactggacagggtctaccccagcttatggctactggttcaaagcagtgactgagacaaccaagggtgctcctgtggccacaaaccaccagagtcgagaggtggaaatgagcacccggggccgattccagctcactggggatcccgccaaggggaactgctccttggtgatcagagacgcgcagatgcaggatgagtcacagtacttctttcgggtggagagaggaagctatgtgagatataatttcatgaacgatgggttctttctaaaagtaacagccctgactcagaagcctgatgtctacatccccgagaccctggagcccgggcagccggtgacggtcatctgtgtgtttaactgggcctttgaggaatgtccacccccttctttctcctggacgggggctgccctctcctcccaaggaaccaaaccaacgacctcccacttctcagtgctcagcttcacgcccagaccccaggaccacaacaccgacctcacctgccatgtggacttctccagaaagggtgtgagcgcacagaggaccgtccgactccgtgtggcctatgcccccagagaccttgttatcagcatttcacgtgacaacacgccagccctggagccccagccccagggaaatgtcccatacctggaagcccaaaaaggccagttcctgcggctcctctgtgctgctgacagccagccccctgccacactgagctgggtcctgcagaacagagtcctctcctcgtcccatccctggggccctagacccctggggctggagctgcccggggtgaaggctggggattcagggcgctacacctgccgagcggagaacaggcttggctcccagcagcgagccctggacctctctggcggaggtggaggccccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtgggtacccatacgatgttccagattacgcttga

SEQ ID NO: 115 - DNA, 1182 bp, Artificial sequence
  synthetic sequence
atggccttcagcggttcccaggctccctacctgagtccagctgtccccttttctgggactattcaaggaggtctccaggacggacttcagatcactgtcaatgggaccgttctcagctccagtggaaccaggtttgctgtgaactttcagactggcttcagtggaaatgacattgccttccacttcaaccctcggtttgaagatggagggtacgtggtgtgcaacacgaggcagaacggaagctgggggcccgaggagaggaagacacacatgcctttccagaaggggatgccctttgacctctgcttcctggtgcagagctcagatttcaaggtgatggtgaacgggatcctcttcgtgcagtacttccaccgcgtgcccttccaccgtgtggacaccatctccgtcaatggctctgtgcagctgtcctacatcagcttccagggcggaggtggaggccccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtgcaccctgcccccatcccgggatgagctgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtgggtacccatacgatgttccagattacgcttga

SEQ ID NO: 116 - DNA, 1431 bp, Artificial sequence
  synthetic sequence
atgggatggtcatgtatcatcctttttctagtagcaactgcaaccggtgtacattcccaggtacaactgcagcagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgcagacaaatcctccagcacagcctacatgcagctcagcagcctgacatctgaggactctgcggtctattactgtgcaagatcgacttactacggcggtgactggtacttcaatgtctggggcgcagggaccacggtcaccgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgggcatcaccaccatcaccattga

SEQ ID NO: 117 - DNA, 1431 bp, Artificial sequence
  synthetic sequence
atgggatggtcatgtatcatcctttttctagtagcaactgcaaccggtgtacattccgaagttcaactggtggagagcggcggggggcttgtgaaaccaggcgggtccctgaaactgagctgtgctgcgagcggctacaccttcacaagctacgttatgcattgggtgaggcaggcacctggtaagggtcttgagtggatcggctatataaatccctacaatgatggcacgaagtacaacgagaaattccaagggcgcgtgactatttctagcgataagtcaatctctactgcctatatggaactttcttctttgagaagcgaagacacagctatgtactactgcgcccgaggcacctactactatggaacccgcgtatttgattattgggggcaaggcaccttggtgacagtaagctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgggcatcaccaccatcaccattga

SEQ ID NO: 118 - DNA, 717 bp, Artificial sequence
  synthetic sequence
atgggatggtcatgtatcatcctttttctagtagcaactgcaaccggtgtacattccgatatcgtaatgacgcaatccccagcgacgctcagtctcagccccggcgaacgggcgactctttcatgtaggagctccaaaagcctccagaatgtcaacggtaatacgtacctttactggtttcaacagaagcctggtcagagtccacaacttctgatataccgcatgtctaatctgaatagtggcgtcccggacagattttctggaagcggcagcggaaccgaatttactctcacgatcagttcactcgaacctgaggacttcgccgtttactactgtatgcaacatctcgagtatcctatcacgtttggagctgggacgaaattggaaatcaagcgcaccgtcgccgcgcccagcgtgttcatcttcccgcccagcgacgagcagctgaagagcggcacggccagcgtggtctgcctgctcaacaacttctacccccgggaggccaaggtgcagtggaaggtggacaacgccctgcagtcggggaacagccaggagagcgtcaccgagcaggacagcaaggacagcacctacagcctgtcgagcaccctcacgctgagcaaggccgactacgagaagcacaaggtgtacgcgtgcgaggtgacccaccagggcctgagcagccccgtcaccaagtcgttcaaccgcggagaatgctga

SEQ ID NO: 119 - DNA, 1422 bp, Artificial sequence
  synthetic sequence
atgggatggtcatgtatcatcctttttctagtagcaactgcaaccggtgtacattccgaggtgcagctggtggagtctgggggaggcttggtccagcctggggggtccctgagactctcctgtgcagcctctggattcacctttagtggctatggcatgagctgggtccgccaggctccagggaaggggctggagtgggtggccaccataactagtggtggaacttacacctactatccagactctgtgaagggccgattcaccatctccagagacaacgccaagaactcactgtatctgcaaatgaacagcctgagagccgaggacacggctgtgtattactgtgcgagatccctcgcgggaaatgctatggactactggggccaaggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgggcatcaccaccatcaccattga

SEQ ID NO: 120 - DNA, 702 bp, Artificial sequence
  synthetic sequence
atgggatggtcatgtatcatcctttttctagtagcaactgcaaccggtgtacattccgaaattgtgttgacacagtctccagccaccctgtctttgtctccaggggaaagagccaccctctcctgcagggccagtcagactattagcgactacttacactggtaccaacagaaacctggccaggctcccaggctcctcatcaaatttgcatcccaatccatttctggcatcccagccaggttcagtggcagtgggtctgggacagacttcactctcaccatcagcagcctagagcctgaagattttgcagtttattactgtcagaatggtcacggctttcctcggacgttcggccaagggaccaaggtggaaatcaaacgcaccgtcgccgcgcccagcgtgttcatcttcccgcccagcgacgagcagctgaagagcggcacggccagcgtggtctgcctgctcaacaacttctacccccgggaggccaaggtgcagtggaaggtggacaacgccctgcagtcggggaacagccaggagagcgtcaccgagcaggacagcaaggacagcacctacagcctgtcgagcaccctcacgctgagcaaggccgactacgagaagcacaaggtgtacgcgtgcgaggtgacccaccagggcctgagcagccccgtcaccaagtcgttcaaccgcggagaatgctga

SEQ ID NO: 121 - DNA, 1419 bp, Artificial sequence
  synthetic sequence
atgggatggtcatgtatcatcctttttctagtagcaactgcaaccggtgtacattcccaagttcaacttcaacaacccggcgcagagctggttaagcctggggcgagtgtaatgatgagttgcaaagcaagcggatacacgtttaccaactacaatatgcattgggtaaaacagacgcctgggcaaggtcttgaatggatcggcaccatctaccctggcaacgatgatacgtcatacaaccagaagtttaaggataaggccacgctgaccgccgataaatcctcctctgcagcgtacatgcagttgtctagtcttacgtctgaggactctgcggtgtactattgcgccagggggggttatagagctatggactactgggggcaagggacctccgtgaccgtatcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgggcatcaccaccatcaccattga

SEQ ID NO: 122 - DNA, 717 bp, Artificial sequence
  synthetic sequence
atgggatggtcatgtatcatcctttttctagtagcaactgcaaccggtgtacattccgatgtgttgatgacgcaaaccccattgtctctcccagtaagcctgggcgaccaagcgagtatctcttgccgctcatcccaatcaatcgtttattccaatggcaacacatatctgggatggtacctccaaaaaccagggcaaagcccaaagctgcttatatataaagtgtcaaatcgatttagcggagtcccggacagattttcagggtcagggtccggaacagattttacgttgaaaatctctcgcgtagaagcagaggacctgggggtctatcattgctttcagggtagtcatgtcccatatacctttggcggtggtacaaaggtagaaatcaagcgcaccgtcgccgcgcccagcgtgttcatcttcccgcccagcgacgagcagctgaagagcggcacggccagcgtggtctgcctgctcaacaacttctacccccgggaggccaaggtgcagtggaaggtggacaacgccctgcagtcggggaacagccaggagagcgtcaccgagcaggacagcaaggacagcacctacagcctgtcgagcaccctcacgctgagcaaggccgactacgagaagcacaaggtgtacgcgtgcgaggtgacccaccagggcctgagcagccccgtcaccaagtcgttcaaccgcggagaatgctga

SEQ ID NO: 123 - DNA, 1428 bp, Artificial sequence
  synthetic sequence
atgggatggtcatgtatcatcctttttctagtagcaactgcaaccggtgtacattccgaagttcaattgctggagagcggcggaggtttggtccagcccggcggttctttgcgactgtcctgcgcagcctctggtttcacattttcttcctacattatgatgtgggtgcgccaggctcctggaaagggacttgaatgggtaagcagcatttatccctctggtgggatcactttctacgccgataccgtgaaggggagattcactatatcacgagacaactcaaagaataccctctatcttcagatgaatagtttgcgagccgaggatacggctgtctactactgtgccaggatcaagttgggtaccgtgacaactgttgattattggggtcaaggaactctcgtaaccgtcagcagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgggcatcaccaccatcaccattga

SEQ ID NO: 124 - DNA, 705 bp, Artificial sequence
  synthetic sequence
atgggatggtcatgtatcatcctttttctagtagcaactgcaaccggtgtacattcccagagtgccctgacgcagcctgcttctgtctctgggagtccagggcagtccattaccattagttgtactggtacatcctctgacgtgggtgggtataactatgttagttggtatcaacagcatcccggtaaagctcccaaactgatgatctacgacgtgagtaataggcccagcggtgttagtaacaggttttcaggatctaagagtggtaacacagcgtcattgactataagtgggctgcaagctgaggatgaagctgattactactgctcatcatatacgtcatcaagtacaagagtatttgggaccggtacgaaagtcacagtccttggacagccaaaagccaatccgacagtaactttgttcccgccaagctcagaggagctgcaagcgaataaagcaactcttgtgtgtctgataagtgatttttacccgggagcggtcactgtggcgtggaaggcagacgggtcaccagttaaggctggagtggagaccacgaaaccgagcaagcaaagcaacaacaaatatgccgcatcatcatatctgtctctgacccccgagcagtggaagtcccatcgaagttactcttgccaggtcacccatgagggctctactgttgagaagacggtcgctcccacggagtgttaa

SEQ ID NO: 125 - DNA, 1422 bp, Artificial sequence
  synthetic sequence
atgggatggtcatgtatcatcctttttctagtagcaactgcaaccggtgtacattccgaagtgcaactggtcgagtcagggggcgggcttgttcagccggggggtagcttgcgactgtcctgtgccgcttcaggattcacattcagcgattcctggattcattgggtccggcaagccccagggaagggactcgaatgggtggcatggatttctccttatggcggtagcacctactatgcggactcagtgaaaggacgctttacgatatcagcagatacttcaaaaaatacggcgtatcttcaaatgaattcactccgagctgaggatactgccgtgtattactgcgcgagacggcattggccgggaggttttgattattggggtcagggaaccctcgttaccgtgtcatcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgggcatcaccaccatcaccattga

SEQ ID NO: 126 - DNA, 702 bp, Artificial sequence
  synthetic sequence
atgggatggtcatgtatcatcctttttctagtagcaactgcaaccggtgtacattccgacatacaaatgacccaaagtccaagctctctttcagcatcagttggagatcgcgttactatcacctgccgcgcgagtcaagacgtaagcactgctgtagcatggtatcaacagaagccagggaaggccccaaagctcctgatatacagcgccagtttcctgtatagtggtgtacccagccgattttctggcagtggaagtggcacggactttacactcaccatcagtagcttgcagccagaagattttgcgacgtattactgtcaacaatatttgtaccacccagcgacttttgggcagggaaccaaggtcgagataaagcgcaccgtcgccgcgcccagcgtgttcatcttcccgcccagcgacgagcagctgaagagcggcacggccagcgtggtctgcctgctcaacaacttctacccccgggaggccaaggtgcagtggaaggtggacaacgccctgcagtcggggaacagccaggagagcgtcaccgagcaggacagcaaggacagcacctacagcctgtcgagcaccctcacgctgagcaaggccgactacgagaagcacaaggtgtacgcgtgcgaggtgacccaccagggcctgagcagccccgtcaccaagtcgttcaaccgcggagaatgctga

SEQ ID NO: 127 - DNA, 2799 bp, Artificial sequence
  synthetic sequence
atgcatctgctggggccctggctgctgctgctggtgctggagtacttggctttctctgactcaagtaaatgggtttttgagcaccctgaaaccctctacgcctgggagggggcctgcgtctggatcccctgcacctacagagccctagatggtgacctggaaagcttcatcctgttccacaatcctgagtataacaagaacacctcgaagtttgatgggacaagactctatgaaagcacaaaggatgggaaggttccttctgagcagaaaagggtgcaattcctgggagacaagaataagaactgcacactgagtatccacccggtgcacctcaatgacagtggtcagctggggctgaggatggagtccaagactgagaaatggatggaacgaatacacctcaatgtctctgaaaggccttttccacctcatatccagctccctccagaaattcaagagtcccaggaagtcactctgacctgcttgctgaatttctcctgctatgggtatccgatccaattgcagtggctcctagagggggttccaatgaggcaggctgctgtcacctcgacctccttgaccatcaagtctgtcttcacccggagcgagctcaagttctccccacagtggagtcaccatgggaagattgtgacctgccagcttcaggatgcagatgggaagttcctctccaatgacacggtgcagctgaacgtgaagcacaccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaagtactgctgtcaggtctccaatgacgtgggcccgggaaggtcggaagaagtgttcctgcaagtgcagtatgccccggaaccttccacggttcagatcctccactcaccggctgtggagggaagtcaagtcgagtttctttgcatgtcactggccaatcctcttccaacaaattacacgtggtaccacaatgggaaagaaatgcagggaaggacagaggagaaagtccacatcccaaagatcctcccctggcacgctgggacttattcctgtgtggcagaaaacattcttggtactggacagaggggcccgggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcaaggtgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccgggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagaccatcggcaggcgaggcggaggtggaggccccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtgcaccctgcccccatcccgggatgagctgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtgggtacccatacgatgttccagattacgcttga

SEQ ID NO: 128 - DNA, 1716 bp, Artificial sequence
  synthetic sequence
atgcatctgctggggccctggctgctgctgctggtgctggagtacttggctttctctgactcaagtaaatgggtttttgagcaccctgaaaccctctacgcctgggagggggcctgcgtctggatcccctgcacctacagagccctagatggtgacctggaaagcttcatcctgttccacaatcctgagtataacaagaacacctcgaagtttgatgggacaagactctatgaaagcacaaaggatgggaaggttccttctgagcagaaaagggtgcaattcctgggagacaagaataagaactgcacactgagtatccacccggtgcacctcaatgacagtggtcagctggggctgaggatggagtccaagactgagaaatggatggaacgaatacacctcaatgtctctgaaaggccttttccacctcatatccagctccctccagaaattcaagagtcccaggaagtcactctgacctgcttgctgaatttctcctgctatgggtatccgatccaattgcagtggctcctagagggggttccaatgaggcaggctgctgtcacctcgacctccttgaccatcaagtctgtcttcacccggagcgagctcaagttctccccacagtggagtcaccatgggaagattgtgacctgccagcttcaggatgcagatgggaagttcctctccaatgacacggtgcagctgaacgtgaagcacaccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaagtactgctgtcaggtctccaatgacgtgggcccgggaaggtcggaagaagtgttcctgcaaggcggaggtggaggccccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtgcaccctgcccccatcccgggatgagctgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtgggtacccatacgatgttccagattacgcttga

SEQ ID NO: 129 - DNA, 1755 bp, Artificial sequence
  synthetic sequence
atgctactgccactgctgctgtcctcgctgctgggcgggtcccaggctatggatgggagattctggatacgagtgcaggagtcagtgatggtgccggagggcctgtgcatctctgtgccctgctctttctcctacccccgacaggactggacagggtctaccccagcttatggctactggttcaaagcagtgactgagacaaccaagggtgctcctgtggccacaaaccaccagagtcgagaggtggaaatgagcacccggggccgattccagctcactggggatcccgccaaggggaactgctccttggtgatcagagacgcgcagatgcaggatgagtcacagtacttctttcgggtggagagaggaagctatgtgagatataatttcatgaacgatgggttctttctaaaagtaacagccctgactcagaagcctgatgtctacatccccgagaccctggagcccgggcagccggtgacggtcatctgtgtgtttaactgggcctttgaggaatgtccacccccttctttctcctggacgggggctgccctctcctcccaaggaaccaaaccaacgacctcccacttctcagtgctcagcttcacgcccagaccccaggaccacaacaccgacctcacctgccatgtggacttctccagaaagggtgtgagcgcacagaggaccgtccgactccgtgtggcctatgcccccagagaccttgttatcagcatttcacgtgacaacacgccagccctggagccccagccccagggaaatgtcccatacctggaagcccaaaaaggccagttcctgcggctcctctgtgctgctgacagccagccccctgccacactgagctgggtcctgcagaacagagtcctctcctcgtcccatccctggggccctagacccctggggctggagctgcccggggtgaaggctggggattcagggcgctacacctgccgagcggagaacaggcttggctcccagcagcgagccctggacctctctggcggaggtggaggccccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtgcaccctgcccccatcccgggatgagctgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtgggtacccatacgatgttccagattacgcttga

SEQ ID NO: 130 - DNA, 1641 bp, Artificial sequence
  synthetic sequence
atgattgcttcacagtttctctcagctctcactttggtgcttctcattaaagagagtggagcctggtcttacaacacctccacggaagctatgacttatgatgaggccagtgcttattgtcagcaaaggtacacacacctggttgcaattcaaaacaaagaagagattgagtacctaaactccatattgagctattcaccaagttattactggattggaatcagaaaagtcaacaatgtgtgggtctgggtaggaacccagaaacctctgacagaagaagccaagaactgggctccaggtgaacccaacaataggcaaaaagatgaggactgcgtggagatctacatcaagagagaaaaagatgtgggcatgtggaatgatgagaggtgcagcaagaagaagcttgccctatgctacacagctgcctgtaccaatacatcctgcagtggccacggtgaatgtgtagagaccatcaataattacacttgcaagtgtgaccctggcttcagtggactcaagtgtgagcaaattgtgaactgtacagccctggaatcccctgagcatggaagcctggtttgcagtcacccactgggaaacttcagctacaattcttcctgctctatcagctgtgataggggttacctgccaagcagcatggagaccatgcagtgtatgtcctctggagaatggagtgctcctattccagcctgcaatgtggttgagtgtgatgctgtgacaaatccagccaatgggttcgtggaatgtttccaaaaccctggaagcttcccatggaacacaacctgtacatttgactgtgaagaaggatttgaactaatgggagcccagagccttcagtgtacctcatctgggaattgggacaacgagaagccaacgtgtaaagctggcggaggtggaggccccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtgcaccctgcccccatcccgggatgagctgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtgggtacccatacgatgttccagattacgcttga

SEQ ID NO: 131 - DNA, 1701 bp, Artificial sequence
  synthetic sequence
atggccaactgccaaatagccatcttgtaccagagattccagagagtggtctttggaatttcccaactcctttgcttcagtgccctgatctctgaactaacaaaccagaaagaagtggcagcatggacttatcattacagcacaaaagcatactcatggaatatttcccgtaaatactgccagaatcgctacacagacttagtggccatccagaataaaaatgaaattgattacctcaataaggtcctaccctactacagctcctactactggattgggatccgaaagaacaataagacatggacatgggtgggaaccaaaaaggctctcaccaacgaggctgagaactgggctgataatgaacctaacaacaaaaggaacaacgaggactgcgtggagatatacatcaagagtccgtcagcccctggcaagtggaatgatgagcactgcttgaagaaaaagcacgcattgtgttacacagcctcctgccaggacatgtcctgcagcaaacaaggagagtgcctcgagaccatcgggaactacacctgctcctgttaccctggattctatgggccagaatgtgaatacgtgagagagtgtggagaacttgagctccctcaacacgtgctcatgaactgcagccaccctctgggaaacttctcttttaactcgcagtgcagcttccactgcactgacgggtaccaagtaaatgggcccagcaagctggaatgcttggcttctggaatctggacaaataagcctccacagtgtttagctgcccagtgcccacccctgaagattcctgaacgaggaaacatgacctgccttcattctgcaaaagcattccagcatcagtctagctgcagcttcagttgtgaagagggatttgcattagttggaccggaagtggtgcaatgcacagcctcgggggtatggacagccccagccccagtgtgtaaagctggcggaggtggaggccccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtgcaccctgcccccatcccgggatgagctgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtgggtacccatacgatgttccagattacgcttga

SEQ ID NO: 132 - DNA, 1425 bp, Artificial sequence
  synthetic sequence
atgggatggtcatgtatcatcctttttctagtagcaactgcaaccggtgtacattcccaagttcaactggtccagagtggaggcggagtagtgcaacccggtaggtcactccggctgagttgtaaggcttcagggtatactttcacgcgctacacgatgcactgggtacgacaagcccccggaaaagggctggaatggataggctacatcaatccgagtagaggctacacaaactataatcagaaggtcaaagataggtttactatttcccgcgataacagcaaaaatacggcttttctccaaatggactctttgaggcccgaagacactggggtttatttttgtgccaggtattacgatgaccactattgtttggactactggggacagggcaccccagtcacggttagcagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgggcatcaccaccatcaccattga

SEQ ID NO: 133 - DNA, 699 bp, Artificial sequence
  synthetic sequence
atgggatggtcatgtatcatcctttttctagtagcaactgcaaccggtgtacattccgatatacaaatgacccaatcaccatctagcctctctgcctcagtaggtgaccgggttacgataacctgttctgcctcctctagcgtctcctatatgaattggtatcaacaaacaccaggcaaagcgcccaaacgatggatctacgacacgtccaagttggcatctggagtgccctcacgcttctcaggaagcggatcagggacggattacacctttaccattagcagcctgcaaccagaggacattgcaacttattattgccagcaatggtcatcaaatccattcaccttcggtcaaggcactaagctccagataactcgcaccgtcgccgcgcccagcgtgttcatcttcccgcccagcgacgagcagctgaagagcggcacggccagcgtggtctgcctgctcaacaacttctacccccgggaggccaaggtgcagtggaaggtggacaacgccctgcagtcggggaacagccaggagagcgtcaccgagcaggacagcaaggacagcacctacagcctgtcgagcaccctcacgctgagcaaggccgactacgagaagcacaaggtgtacgcgtgcgaggtgacccaccagggcctgagcagccccgtcaccaagtcgttcaaccgcggagaatgctga

SEQ ID NO: 134 - DNA, 1425 bp, Artificial sequence; identical to SEQ ID NO: 132
  synthetic sequence

SEQ ID NO: 135 - DNA, 699 bp, Artificial sequence; identical to SEQ ID NO: 133
  synthetic sequence

SEQ ID NO: 136 - DNA, 1194 bp, Artificial sequence
  synthetic sequence
atgctgctgctgctgctgcccctgctctgggggagggagagggcggaaggacaggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgacggcggaggtggaggccccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtgggtacccatacgatgttccagattacgcttga

SEQ ID NO: 137 - DNA, 1539 bp, Artificial sequence
  synthetic sequence
atgctgctgctgctgctgcccctgctctgggggagggagagggcggaaggacaggcagacaatttttcgctccatgatgcgttatctgggtctggaaacccaaaccctcaaggatggcctggcgcatgggggaaccagcctgctggggcagggggctacccaggggcttcctatcctggggcctaccccgggcaggcacccccaggggcttatcctggacaggcacctccaggcgcctaccatggagcacctggagcttatcccggagcacctgcacctggagtctacccagggccacccagcggccctggggcctacccatcttctggacagccaagtgcccccggagcctaccctgccactggcccctatggcgcccctgctgggccactgattgtgccttataacctgcctttgcctgggggagtggtgcctcgcatgctgataacaattctgggcacggtgaagcccaatgcaaacagaattgctttagatttccaaagagggaatgatgttgccttccactttaacccacgcttcaatgagaacaacaggagagtcattgtttgcaatacaaagctggataataactggggaagggaagaaagacagtcggttttcccatttgaaagtgggaaaccattcaaaatacatgtactggttgaacctgaccacttcaaggttgcagtgaatgatgctcacttgttgcagtacaatcatcgggttaaaaaactcaatgaaatcagcaaactgggaatttctggtgacatagacctcaccagtgcttcatataccatgataggcggaggtggaggccccaaatcttgtgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtgggtacccatacgatgttccagattacgcttga

(19) United States
(12) Patent Application Publication
(10) Pub. No.: US 2024/0182584 A1
(43) Pub. Date: Jun. 6, 2024

(54) Bispecific Molecules and Related Compositions and Methods

(71) Applicant: The Board of Trustees of the Leland Stanford Junior University, Stanford, CA (US)

(72) Inventors: Carolyn R. Bertozzi, Menlo Park, CA (US); Jessica Stark, Redwood City, CA (US); Melissa Gray, Stanford, CA (US); Simon Wisnovsky, Redwood City, CA (US)

(21) Appl. No.: 18/284,750

(22) PCT Filed: Apr. 1, 2022
(86) PCT No.: PCT/US2022/023166

§ 371 Date: Sep. 28, 2023

Related U.S. Application Data
(60) Provisional application No. 63/170,297, filed on Apr. 2, 2021.

(51) Int. Cl. C07K 16/28; A61K 39/00; A61P 35/00

(52) U.S. Cl. CPC C07K 16/2854; A61P 35/00; C07K 16/2803; C07K 16/2851; A61K 39/00; C07K 2317/31

(57) ABSTRACT

Aspects of the present disclosure include bispecific molecules. The bispecific molecules comprise a cell-targeting moiety and a glycan-binding moiety. According to some embodiments, the cell-targeting moiety is a cancer cell-targeting moiety or an immune cell-targeting moiety. In certain embodiments, the glycan-binding moiety comprises the sialoglycan-binding domain of a lectin, non-limiting examples of which are sialic acid-binding immunoglobulin-like lectins (Siglecs). The bispecific molecules may take a variety of forms including heterodimeric molecules, fusion proteins, conjugates, and the like. Compositions, kits and methods of using the bifunctional molecules, e.g., for therapeutic purposes, are also provided.

### CROSS-REFERENCE TO RELATED APPLICATIONS

This application claims the benefit of U.S. Provisional Patent Application No. 63/170,297, filed Apr. 2, 2021, which application is incorporated herein by reference in its entirety.

### STATEMENT OF GOVERNMENT SUPPORT

This invention was made with Government support under contracts CA226051, CA250324, and GM058867 awarded by the National Institutes of Health. The Government has certain rights in the invention.

### INCORPORATION BY REFERENCE OF SEQUENCE LISTING PROVIDED AS A TEXT FILE

A Sequence Listing is provided herewith in a text file, (S21-091_STAN-1838WO_SEQ_LIST_ST25), created on Apr. 1, 2022 and having a size of 425,000 bytes of file. The contents of the text file are incorporated herein by reference in its entirety.

### INTRODUCTION

Despite the remarkable benefits of cancer immunotherapies observed in select cases, many tumors remain unresponsive to existing treatments. There is thus an unmet need for therapies targeting additional immune checkpoints that drive cancer progression. Hypersialylation, or upregulation of the sialic acid monosaccharide on cell surfaces is an established hallmark of cancer associated with increased aggressiveness and rates of metastasis. Emerging evidence suggests that hypersialylation allows tumors to engage inhibitory glycan-binding receptors called Siglecs on immune cells. Siglec receptors are expressed by every immune cell class and the intracellular domains of eight of the Siglec family members bear homology to the established PD-1 immune checkpoint. These studies have established Siglecs and their sialoglycan ligands as immune checkpoints that contribute to cancer progression.

The discovery and characterization of Siglec-sialoglycan immune checkpoints has spurred interest in targeting Siglec receptors for checkpoint blockade. However, the lack of glycan-binding reagents with high affinity and selectivity has prevented targeting of tumor-associated sialoglycan ligands for checkpoint blockade to date. The weak immunogenicity of mammalian glycan structures has historically impeded the development of anti-glycan antibodies. Even if glycan-binding antibodies were available, the identities of sialoglycans used by tumors to engage Siglecs are not fully understood, precluding their use as targets. Soluble Siglec-Fc chimeras have been shown to maintain native sialoglycan binding specificities, but their binding affinities are too low to be used as decoy-receptor therapeutics. Agents effective for targeting tumor-associated sialoglycans for checkpoint blockade are therefore needed.

### SUMMARY

Aspects of the present disclosure include bispecific molecules. The bispecific molecules comprise a cell-targeting moiety and a glycan-binding moiety. According to some embodiments, the cell-targeting moiety is a cancer cell-targeting moiety or an immune cell-targeting moiety. In certain embodiments, the glycan-binding moiety comprises the sialoglycan-binding domain of a lectin, non-limiting examples of which are sialic acid-binding immunoglobulin-like lectins (Siglecs). The bispecific molecules may take a variety of forms including heterodimeric molecules, fusion proteins, conjugates, and the like. Compositions, kits and methods of using the bifunctional molecules, e.g., for therapeutic purposes, are also provided.

### BRIEF DESCRIPTION OF THE FIGURES

FIG. 1a-1e: Schematic illustrations and data demonstrating that antibody-lectin (AbLec) bispecifics enable use of lectin decoy receptors for checkpoint blockade.

FIG. 2a-2f: Schematic illustrations and data demonstrating that AbLecs block binding of targeted glycan-binding immunoreceptors.

FIG. 3a-3e: Schematic illustrations and data demonstrating that AbLecs enhance antibody-dependent cellular phagocytosis and cytotoxicity in vitro.

FIG. 4: Data demonstrating that AbLec enhancement of in vitro ADCP is dependent on expression of the targeted antigen (in this example, HER2 targeted by the trastuzumab arm).

FIG. 5: Data demonstrating that AbLecs bind to human tumor cell lines and block Siglec receptor binding.

FIG. 6a-6d: Schematic illustrations and data demonstrating that AbLecs outperform combination immunotherapy via Siglec-dependent enhancement of anti-tumor immune responses in vitro.

FIG. 7a-7d: Schematic illustrations and data demonstrating that the AbLec platform enables blockade of diverse glyco-immune checkpoint targets.

FIG. 8: Data demonstrating that R7 AbLecs enhance ADCC of CD20+ Raji cells compared to rituximab.

FIG. 9: Schematic illustrations and data demonstrating the expression of diverse AbLec molecules.

FIG. 10: Schematic illustration of AbLecs as modular agents for targeting glycan immune checkpoints.

FIG. 11: The amino acid sequence of an example Rituximab-Siglec-7 AbLec heterodimer (“Ritux-Sig7 AbLec”) including knobs-into-holes modified CH3 domains to facilitate heterodimer formation as confirmed by mass spectrometry.

FIG. 12: The amino acid sequence of an example Rituximab-Siglec-9 AbLec heterodimer (“Ritux-Sig9 AbLec”) including knobs-into-holes modified CH3 domains to facilitate heterodimer formation as confirmed by mass spectrometry.

FIG. 13: The amino acid sequence of an example Trastuzumab-Siglec-9 AbLec heterodimer (“Tras-Sig9 AbLec”) including knobs-into-holes modified CH3 domains to facilitate heterodimer formation as confirmed by mass spectrometry.

FIG. 14: The amino acid sequence of an example Trastuzumab-Siglec-7 AbLec heterodimer (“Tras-Sig7 AbLec”) including knobs-into-holes modified CH3 domains to facilitate heterodimer formation as confirmed by mass spectrometry.

### DETAILED DESCRIPTION

Before the bispecific molecules, compositions and methods of the present disclosure are described in greater detail, it is to be understood that the bispecific molecules, compositions and methods are not limited to particular embodiments described, as such may, of course, vary. It is also to be understood that the terminology used herein is for the purpose of describing particular embodiments only, and is not intended to be limiting, since the scope of the bispecific molecules, compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the bispecific molecules, compositions and methods. The upper and lower limits of these smaller ranges may independently be included in the smaller ranges and are also encompassed within the bispecific molecules, compositions and methods, subject to any specifically excluded limit in the stated range. Where the stated range includes one or both of the limits, ranges excluding either or both of those included limits are also included in the bispecific molecules, compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the bispecific molecules, compositions and methods belong. Although any bispecific molecules, compositions and methods similar or equivalent to those described herein can also be used in the practice or testing of the bispecific molecules, compositions and methods, representative illustrative bispecific molecules, compositions and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present bispecific molecules, compositions and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the bispecific molecules, compositions and methods, which are, for clarity, described in the context of separate embodiments, may also be provided in combination in a single embodiment. Conversely, various features of the bispecific molecules, compositions and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present bispecific molecules, compositions and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Bispecific Molecules

The present disclosure provides bispecific molecules. The bispecific molecules comprise a cell-targeting moiety (e.g., a cancer cell-targeting moiety or an immune cell-targeting moiety) and a glycan-binding moiety. In certain embodiments, the glycan-binding moiety comprises the sialoglycan-binding domain of a lectin, non-limiting examples of which are sialic acid-binding immunoglobulin-like lectins (Siglecs). The bispecific molecules may take a variety of forms including heterodimeric molecules, fusion proteins, conjugates, and the like. As demonstrated herein, the bispecific molecules of the present disclosure comprising cancer cell-targeting moieties and glycan-binding moieties are effective in enhancing anti-tumor immune responses, e.g., by enhanced antibody-dependent cellular phagocytosis (ADCP) and/or cytotoxicity (ADCC). Moreover, the bispecific format was required for the enhancement, and the results demonstrate a therapeutic synergy that arises from combining the tumor cell-targeting and glycan-binding arms in a single bispecific molecule. As used herein, “synergy” or “synergistic effect” with regard to an effect produced by two or more individual components refers to a phenomenon in which the total effect produced by these components, when utilized in combination (here, present in a single bispecific molecule), is greater than the sum of the individual effects of each component acting alone. Further details regarding bispecific molecules according to embodiments of the present disclosure will now be described.

### Cell-Targeting Moieties

A variety of cell-targeting moieties may be employed in the bispecific molecules of the present disclosure. In certain embodiments, the cell-targeting moiety is a cancer cell-targeting moiety. By “cancer cell” is meant a cell exhibiting a neoplastic cellular phenotype, which may be characterized by one or more of the following exemplary characteristics: abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a hematological malignancy (e.g., a leukemia cell, a lymphoma cell, a myeloma cell, etc.), a primary tumor, a metastatic tumor, and the like.

In certain embodiments, when the cell-targeting moiety is a cancer cell-targeting moiety, the cancer cell-targeting moiety specifically binds to a molecule (e.g., a protein) expressed on the surface of a cancer cell. Non-limiting examples of cancer cell surface molecules to which the cancer cell-targeting moiety may specifically bind include 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD20, CD22, CD25, CD27L, CD30, CD33, CD37, CD44, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvIII, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), GD2 ganglioside, glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), programmed cell death receptor ligand 1 (PD-L1), programmed cell death receptor ligand 2 (PD-L2), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), Tn antigen, trophoblast cell-surface antigen (TROP-2), and Wilms' tumor 1 (WT1).

According to some embodiments, the cell-targeting moiety is an immune cell-targeting moiety. In certain embodiments, when the cell-targeting moiety is an immune cell-targeting moiety, the immune cell-targeting moiety specifically binds to a molecule (e.g., a protein) expressed on the surface of an immune cell. The immune cell-targeting moiety may be selected to target any desired immune cell, non-limiting examples of which include T cells, B cells, natural killer (NK) cells, a macrophages, monocytes, neutrophils, dendritic cells, mast cells, basophils, and eosinophils. In some embodiments, the immune cells are T cells. Exemplary T cell types include naive T cells (T_{N}), cytotoxic T cells (T_{CTL}), memory T cells (T_{MEM}), T memory stem cells (T_{SCM}), central memory T cells (T_{CM}), effector memory T cells (T_{EM}), tissue resident memory T cells (T_{RM}), effector T cells (T_{EFF}), regulatory T cells (T_{REGs}), helper T cells (T_{H}, T_{H}1, T_{H}2, T_{H}17) CD4+ T cells, CD8+ T cells, virus-specific T cells, alpha beta T cells (T_{αβ}), and gamma delta T cells (T_{γδ}).

Non-limiting examples of immune cell surface molecules to which the immune cell-targeting moiety may specifically bind include PD-1, PD-L1, PD-L2, CLTA-4, VISTA, LAG-3, TIM-3, CD24, CD47, SIRPalpha, CD3, CD8, CD4, CD28, CD80, CD86, CD19, ICOS, OX40, OX40L, GD3 ganglioside, TIGIT, Siglec-2, Siglec-3, Siglec-7, Siglec-8, Siglec-9, Siglec-10, Siglec-15, galectin-9, B7-H3, B7-H4, CD40, CD40L, B7RP1, CD70, CD27, BTLA, HVEM, KIR, 4-1BB, 4-1BBL, CD226, CD155, CD112, GITR, GITRL, A2aR, CD137, CD137L, CD45, CD206, CD163, TRAIL, NKG2D, CD16, and TGF-beta.

According to some embodiments, the cell-targeting moiety comprises a small molecule that binds to a cell surface molecule on a target cell. By “small molecule” is meant a compound having a molecular weight of 1000 atomic mass units (amu) or less. In certain embodiments, the small molecule is 750 amu or less, 500 amu or less, 400 amu or less, 300 amu or less, or 200 amu or less. According to some embodiments, the small molecule is not made of repeating molecular units such as are present in a polymer. In certain embodiments, the target cell surface molecule is a receptor for which the ligand is a small molecule, and the small molecule of the cell-targeting moiety is the small molecule ligand (or a derivative thereof) of the receptor. Small molecules that find use in targeting a conjugate to a target cell of interest are known. As just one example, folic acid (FA) derivatives have been shown to effectively target certain types of cancer cells by binding to the folate receptor, which is overexpressed, e.g., in many epithelial tumors. See, e.g., Vergote et al. (2015) Ther. Adv. Med. Oncol. 7(4):206-218. In another example, the small molecule sigma-2 has proven to be effective in targeting cancer cells. See, e.g., Hashim et al. (2014) Molecular Oncology 8(5):956-967. Sigma-2 is the small molecule ligand for sigma-2 receptors, which are overexpressed in many proliferating tumor cells including pancreatic cancer cells. In certain aspects, the cell-targeting moiety of a bispecific molecule of the present disclosure comprises a small molecule, in which it has been demonstrated in the context of a small molecule drug conjugate (SMDC) that the small molecule is effective at targeting a conjugate to a target cell of interest by binding to a cell surface molecule on the target cell.

According to certain embodiments, the cell-targeting moiety comprises a ligand. As used herein, a “ligand” is a substance that forms a complex with a biomolecule to serve a biological purpose. The ligand may be a substance selected from a circulating factor, a secreted factor, a cytokine, a growth factor, a hormone, a peptide, a polypeptide, a small molecule, and a nucleic acid, that forms a complex with the cell surface molecule on the surface of the target cell. In certain embodiments, when the cell-targeting moiety comprises a ligand, the ligand is modified in such a way that complex formation with the cell surface molecule occurs, but the normal biological result of such complex formation does not occur. In certain aspects, the ligand is the ligand of a cell surface receptor present on a target cell.

In certain embodiments, the cell-targeting moiety comprises an aptamer. By “aptamer” is meant a nucleic acid (e.g., an oligonucleotide) that has a specific binding affinity for a target cell surface molecule. Aptamers exhibit certain desirable properties for targeted delivery of the bispecific molecule, such as ease of selection and synthesis, high binding affinity and specificity, low immunogenicity, and versatile synthetic accessibility. Aptamers that bind to cell surface molecules are known and include, e.g., TTA1 (a tumor targeting aptamer to the extracellular matrix protein tenascin-C). Aptamers that find use in the bispecific molecules of the present disclosure include those described in Zhu et al. (2015) ChemMedChem 10(1):39-45; Sun et al. (2014) Mol. Ther. Nucleic Acids 3:e182; and Zhang et al. (2011) Curr. Med. Chem. 18(27):4185-4194.

According to some embodiments, the cell-targeting moiety comprises a nanoparticle. As used herein, a “nanoparticle” is a particle having at least one dimension in the range of from 1 nm to 1000 nm, from 20 nm to 750 nm, from 50 nm to 500 nm, including 100 nm to 300 nm, e.g., 120-200 nm. The nanoparticle may have any suitable shape, including but not limited to spherical, spheroid, rod-shaped, disk-shaped, pyramid-shaped, cube-shaped, cylinder-shaped, nanohelical-shaped, nanospring-shaped, nanoring-shaped, arrow-shaped, teardrop-shaped, tetrapod-shaped, prism-shaped, or any other suitable geometric or non-geometric shape. In certain embodiments, the nanoparticle includes on its surface one or more of the other targeting moieties described herein, e.g., antibodies, ligands, aptamers, small molecules, etc. Nanoparticles that find use in the bispecific molecules of the present disclosure include those described in Wang et al. (2010) Pharmacol. Res. 62(2):90-99; Rao et al. (2015) ACS Nano 9(6):5725-5740; and Byrne et al. (2008) Adv. Drug Deliv. Rev. 60(15):1615-1626.

In some embodiments the cell targeting moiety specifically binds a receptor expressed on the surface of a target cell. Such a cell-targeting moiety may comprise, e.g., an antigen-binding domain of an antibody that specifically binds the receptor, or a ligand for the receptor. Non-limiting examples of such cell surface receptors include stem cell receptors, immune cell receptors (e.g., T cell receptors, B cell receptors, and the like), growth factor receptors, cytokine receptors, hormone receptors, receptor tyrosine kinases, immune receptors such as CD28, CD80, ICOS, CTLA4, PD1, PD-L1, BTLA, HVEM, CD27, 4-1BB, 4-1BBL, OX40, OX40L, DR3, GITR, CD30, SLAM, CD2, 2B4, TIM1, TIM2, TIM3, TIGIT, CD226, CD160, LAG3, LAIR1, B7-1, B7-H1, and B7-H3, a type I cytokine receptor such as Interleukin-1 receptor, Interleukin-2 receptor, Interleukin-3 receptor, Interleukin-4 receptor, Interleukin-5 receptor, Interleukin-6 receptor, Interleukin-7 receptor, Interleukin-9 receptor, Interleukin-11 receptor, Interleukin-12 receptor, Interleukin-13 receptor, Interleukin-15 receptor, Interleukin-18 receptor, Interleukin-21 receptor, Interleukin-23 receptor, Interleukin-27 receptor, Erythropoietin receptor, GM-CSF receptor, G-CSF receptor, Growth hormone receptor, Prolactin receptor, Leptin receptor, Oncostatin M receptor, Leukemia inhibitory factor, a type II cytokine receptor such as interferon-alpha/beta receptor, interferon-gamma receptor, Interferon type III receptor, Interleukin-10 receptor, Interleukin-20 receptor, Interleukin-22 receptor, Interleukin-28 receptor, a receptor in the tumor necrosis factor receptor superfamily such as Tumor necrosis factor receptor 2 (1B), Tumor necrosis factor receptor 1, Lymphotoxin beta receptor, OX40, CD40, Fas receptor, Decoy receptor 3, CD27, CD30, 4-1BB, Decoy receptor 2, Decoy receptor 1, Death receptor 5, Death receptor 4, RANK, Osteoprotegerin, TWEAK receptor, TACI, BAFF receptor, Herpesvirus entry mediator, Nerve growth factor receptor, B-cell maturation antigen, Glucocorticoid-induced TNFR-related, TROY, Death receptor 6, Death receptor 3, Ectodysplasin A2 receptor, a chemokine receptor such as CCR1, CCR2, CCR3, CCR4, CCR5, CCR6, CCR7, CCR8, CCR9, CCR10, CXCR1, CXCR2, CXCR3, CXCR4, CXCR5, CXCR6, CX3CR1, XCR1, ACKR1, ACKR2, ACKR3, ACKR4, CCRL2, a receptor in the epidermal growth factor receptor (EGFR) family, a receptor in the fibroblast growth factor receptor (FGFR) family, a receptor in the vascular endothelial growth factor receptor (VEGFR) family, a receptor in the rearranged during transfection (RET) receptor family, a receptor in the Eph receptor family, a receptor that can induce cell differentiation (e.g., a Notch receptor), a cell adhesion molecule (CAM), an adhesion receptor such as integrin receptor, cadherin, selectin, and a receptor in the discoidin domain receptor (DDR) family, transforming growth factor beta receptor 1, and transforming growth factor beta receptor 2. In some embodiments, such a receptor is an immune cell receptor selected from a T cell receptor, a B cell receptor, a natural killer (NK) cell receptor, a macrophage receptor, a monocyte receptor, a neutrophil receptor, a dendritic cell receptor, a mast cell receptor, a basophil receptor, and an eosinophil receptor.

In certain embodiments, the cell-targeting moiety comprises an antigen-binding domain of an antibody. By “antibody” is meant an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the cell surface molecule of the target cell, including, but not limited to single chain Fv (scFv), Fab, (Fab′)₂, (scFv′)₂, and diabodies; chimeric antibodies; monoclonal antibodies, human antibodies, humanized antibodies (e.g., humanized whole antibodies, humanized half antibodies, or humanized antibody fragments, e.g., humanized scFv); and fusion proteins comprising an antigen-binding portion of an antibody and a non-antibody protein. In certain embodiments, the antibody is selected from an IgG, Fv, single chain antibody, scFv, Fab, F(ab′)₂, or Fab′. The antibody may be detectably labeled, e.g., with an in vivo imaging agent, a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like.

According to some embodiments, when the cell-targeting moiety comprises an antigen-binding domain of an antibody, the antigen-binding domain is of an antibody approved by the United States Food and Drug Administration and/or the European Medicines Agency (EMA) for use as a therapeutic antibody, e.g., for inducing antibody-dependent cellular cytotoxicity (ADCC), inducing antibody-dependent cellular phagocytosis (ADCP), and/or the like, of certain disease-associated cells in a patient, etc. Non-limiting examples of antigen-binding domains which may be employed in the bispecific molecules of the present disclosure include those from an antibody selected from Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sofituzumab, Catumaxomab, Ertumaxomab, Depatuxizumab, Ontuxizumab, Blontuvetmab, Tamtuvetmab, or an antigen-binding variant thereof, e.g., a single-chain version (e.g., an scFv version).

In certain embodiments, the cell-targeting moiety comprises an antibody heavy chain comprising a γ, α, δ, ε, or μ antibody heavy chain or fragment thereof. According to some embodiments, the antibody heavy chain or fragment thereof is an IgG heavy chain or fragment thereof, e.g., a human IgG1 heavy chain or fragment thereof. In certain embodiments, the antibody heavy chain or fragment thereof comprises a heavy chain variable region (V_{H}). Such an antibody heavy chain or fragment thereof may further include a heavy chain constant region or fragment thereof. For example, when a heavy chain constant region or fragment thereof is included in the cell-targeting moiety, the antibody heavy chain constant region or fragment thereof may include one or more of a C_{H}1 domain, C_{H}2 domain, and/or C_{H}3 domain. According to some embodiments, the cell-targeting moiety comprises a full-length antibody heavy chain—that is, an antibody heavy chain that includes a V_{H}, a C_{H}1 domain, a C_{H}2 domain, and a C_{H}3 domain.

In certain embodiments, the cell-targeting moiety comprises an antibody light chain or fragment thereof. According to some embodiments, the antibody light chain or fragment thereof comprises a kappa (κ) light chain or fragment thereof or a lambda (λ) light chain or fragment thereof. According to some embodiments, the antibody light chain or fragment thereof includes a light chain variable region (V_{L}). Such an antibody light chain or fragment thereof may further include an antibody light chain constant region (C_{L}) or fragment thereof. In certain embodiments, the cell-targeting moiety comprises a full-length antibody light chain—that is, an antibody light chain that includes a V_{L} and a C_{L}.

According to some embodiments, the cell-targeting moiety comprises an antibody heavy chain comprising a variable heavy chain (V_{H}) region and an antibody light chain comprising a variable light chain (V_{L}) region. In certain embodiments, the cell-targeting moiety comprises a C_{H}1 domain, a hinge region, a C_{H}2 domain, a C_{H}3 domain, or any combination thereof. For example, the cell-targeting moiety may comprise an antibody heavy chain comprising a C_{H}2 domain, a C_{H}3 domain, or both. Examples of such cell-targeting moieties include those that comprise a fragment crystallizable (Fc) region.

The cell-targeting moieties and/or glycan-binding moieties of the bispecific molecules of the present disclosure may specifically bind to their respective targets. As used herein, a cell-targeting moiety or glycan-binding moiety “specifically binds” or “preferentially binds” to a target if it binds with greater affinity, avidity, more readily, and/or with greater duration than it binds to other substances, e.g., in a sample and/or in vivo. In certain embodiments, a cell-targeting moiety or glycan-binding moiety “specifically binds” a target if it binds to or associates with the target with an affinity or Ka (that is, an association rate constant of a particular binding interaction with units of 1/M) of, for example, greater than or equal to about 10⁴ M⁻¹. Alternatively, affinity may be defined as an equilibrium dissociation constant (KD) of a particular binding interaction with units of M (e.g., 10⁻⁵ M to 10⁻¹³ M, or less). In certain aspects, specific binding means the cell-targeting moiety or glycan-binding moiety binds to the target with a KD of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the cell-targeting moiety or glycan-binding moiety for the target can be readily determined using conventional techniques, e.g., by competitive ELISA (enzyme-linked immunosorbent assay), equilibrium dialysis, by using surface plasmon resonance (SPR) technology (e.g., the BIAcore 2000 or BIAcore T200 instrument, using general procedures outlined by the manufacturer); by radioimmunoassay; or the like.

### Glycan-Binding Moieties

A variety of glycan-binding moieties may be employed in the bispecific molecules of the present disclosure. In certain embodiments, the glycan-binding moiety comprises the glycan-binding domain of a lectin. For example, the glycan-binding moiety may comprise the sialoglycan-binding domain of a sialoglycan-binding lectin. Non-limiting examples of sialoglycan-binding moieties include those that comprise the sialoglycan-binding domain of a sialic acid-binding immunoglobulin-like lectin (Siglec).

Siglecs are a family of immunomodulatory receptors whose functions are regulated by their glycan ligands. The Siglec family consists of 15 family members in humans that are expressed on a restricted set of cells in the hematopoietic lineage, with known exceptions including Siglec-4 (MAG) on oligodendrocytes and Schwann cells and Siglec-6 on placental trophoblasts. Through their outermost N-terminal V-set domain, Siglecs recognize sialic acid-containing glycan ligands on glycoproteins and glycolipids with unique, yet overlapping, specificities. Recognition of their ligands can affect cellular signaling through immunoreceptor tyrosine-based inhibitory motifs (ITIMs) on their cytoplasmic tails. For the majority of the Siglecs, these ITIMs have the capacity of recruiting phosphatases, therefore, these members are referred to as inhibitory-type Siglecs. Exceptions include Siglec-1 and MAG, which lack such a motif, and the activatory-type Siglecs (Siglecs-14 to -16), which are associated with immunoreceptor tyrosine-based activatory motif (ITAM)-bearing adapter proteins through a positively charge amino acid in their transmembrane region.

Siglecs can be divided into two groups based on their genetic homology among mammalian species. The first group is present in all mammals and consists of Siglec-1 (Sialoadhesin), Siglec-2 (CD22), Siglec-4, and Siglec-15. The second group consists of the CD33-related Siglecs which include Siglec-3 (CD33), -5, -6, -7, -8, -9, -10, -11, -14 and -16. Monocytes, monocyte-derived macrophages, and monocyte-derived dendritic cells have largely the same Siglec profile, namely high expression of Siglec-3, -7, -9, low Siglec-10 expression and upon stimulation with IFN-α, expression of Siglec-1. In contrast, macrophages have primarily expression of Siglec-1, -3, -8, -9, -11, -15, and -16 depending on their differentiation status. Conventional dendritic cells express Siglec-3, -7, and -9, similar to monocyte-derived dendritic cells, but in addition also express low levels of Siglec-2 and Siglec-15. Plasmacytoid dendritic cells express Siglec-1 and Siglec-5. Downregulation of Siglec-7 and Siglec-9 expression on monocyte-derived dendritic cells is observed after stimulation for 48 hours with LPS, however, on monocyte-derived macrophages Siglec expression is not changed upon LPS triggering. Siglecs are also present on other immune cells, such as B cells, basophils, neutrophils, and NK cells. Further details regarding Siglecs may be found, e.g., in Angata et al. (2015) Trends Pharmacol Sci. 36(10): 645-660; Lübbers et al. (2018) Front. Immunol. 9:2807; Bochner et al. (2016) J Allergy Clin Immunol. 135(3):598-608; and Duan et al. (2020) Annu. Rev. Immunol. 38(1): 365-395; the disclosures of which are incorporated herein by reference in their entireties for all purposes.

The glycan-binding moiety may comprise the sialoglycan-binding domain of any of the 15 human Siglec family members. In certain embodiments, the glycan-binding moiety comprises the sialoglycan-binding domain of a CD33-related Siglec. According to some embodiments, the CD33-related Siglec is Siglec-7 (UniProtKB-Q9Y286). In some embodiments, the glycan-binding moiety binds to a Siglec-7 ligand, where the glycan-binding moiety comprises the amino acid sequence set forth in SEQ ID NO: 15, or a Siglec-7 ligand-binding variant thereof comprising 70% or greater, 75% or greater, 80% or greater, 85% or greater, 90% or greater, 95% or greater, 96% or greater, 97% or greater, 98% or greater, or 99% or greater amino acid sequence identity to the amino acid sequence set forth in SEQ ID NO: 15, or a fragment thereof which retains the ability to bind the Siglec-7 ligand. In certain embodiments, the CD33-related Siglec is Siglec-9 (UniProtKB-Q9Y336). In some embodiments, the glycan-binding moiety binds to a Siglec-9 ligand, where the glycan-binding moiety comprises the amino acid sequence set forth in SEQ ID NO: 21, or a Siglec-9 ligand-binding variant thereof comprising 70% or greater, 75% or greater, 80% or greater, 85% or greater, 90% or greater, 95% or greater, 96% or greater, 97% or greater, 98% or greater, or 99% or greater amino acid sequence identity to the amino acid sequence set forth in SEQ ID NO: 21, or a fragment thereof which retains the ability to bind the Siglec-9 ligand. According to some embodiments, the CD33-related Siglec is Siglec-10 (UniProtKB-Q96LC7). In certain embodiments, the glycan-binding moiety comprises the sialoglycan-binding domain of Siglec-15 (UniProtKB-Q6ZMC9). According to some embodiments, the glycan-binding moiety comprises the sialoglycan-binding domain of a Siglec-like adhesin. See, e.g., Deng et al. (2014) PLOS Pathog 10(12):e1004540, and Bensing et al. (2018) Glycobiology 28(8):601-611, the disclosures of which are incorporated herein by reference in their entireties for all purposes.

In certain embodiments, the glycan-binding moiety comprises the glycan-binding domain of a C-type lectin. The C-type lectins are a superfamily of proteins defined by the presence of at least one C-type lectin-like domain (CTLD) and that recognize a broad repertoire of ligands and regulate a diverse range of physiological functions. Most research attention has focused on the ability of C-type lectins to function in innate and adaptive antimicrobial immune responses, but these proteins are increasingly being recognized to have a major role in autoimmune diseases and to contribute to many other aspects of multicellular existence. The term C-type lectin was introduced to distinguish between Ca²⁺-dependent and Ca²⁺-independent carbohydrate-binding lectins. C-type lectins share at least one carbohydrate recognition domain, which is a compact structural module that contains conserved residue motifs and determines the carbohydrate specificity of the CLR. Of particular interest for their role in coupling both innate and adaptive immunity, are the genes of the Dectin-1 and Dectin-2 families localized on the telomeric region of the natural killer cluster of genes. These two groups of C-type lectins are expressed mostly by cells of myeloid lineage such as monocytes, macrophages, dendritic cells (DCs), and neutrophils. C-type lectins not only serve as antigen-uptake receptors for internalization and presentation to T cells but also trigger multiple signaling pathways leading to NF-κB, type I interferon (IFN), and/or inflammasome activation. This leads, in turn, to the production of pro- or anti-inflammatory cytokines and chemokines, subsequently fine tuning adaptive immune responses. Further details regarding C-type lectins may be found, e.g., in Zelensky et al. (2005) FEBS J. 272:6179-6217; Geijtenbeek & Grinhuis (2009) Nature Reviews Immunology 9:465-479; Brown et al. (2018) Nature Reviews Immunology 18:374-389; Dambuza & Brown (2015) Curr. Opin. Immunol. 32:21-7; and Chiffoleau (2018) Front. Immunol. 9:227; the disclosures of which are incorporated herein by reference in their entireties for all purposes. According to some embodiments, the glycan-binding moiety comprises the glycan-binding domain of a C-type lectin selected from DECTIN-1, lectin-like oxidized low-density lipoprotein receptor-1 (LOX-1), C-type lectin-like receptor-1 (CLEC-1), C-type lectin-like receptor 2 (CLEC-2), myeloid inhibitory C-type lectin-like receptor (MICL), CLEC9A, DC immunoreceptor (DCIR), DECTIN-2, blood DC antigen-2 (BDCA-2), macrophage-inducible C-type lectin (MINCLE), macrophage galactose lectin (MGL), and asialoglycoprotein receptor (ASGPR).

In certain embodiments, the glycan-binding moiety comprises the glycan-binding domain of a selectin. Selectins are C-type transmembrane lectins that mediate leukocyte trafficking and specific adhesive interactions of leukocytes, platelets, and endothelial cells with tumor cells. These lectins are present on endothelial cells (E-Selectin), leukocytes (L-Selectin), and platelets (P-Selectin), and preferentially bind glycans containing SLe^{X} and SLe^{A} glycoepitopes, which are abundantly expressed in several tumor types. In the TME, selectins are functionally relevant in the context of leukocyte recruitment, tumor-promoting inflammation, and acquisition of metastatic potential. P-Selectin (CD62P) is involved in tumor growth and metastasis, as it mediates interactions between activated platelets and cancer cells contributing to tumorigenesis. E-Selectin (CD62E) also play major roles in cancer cell adhesiveness at different events of the metastatic cascade, promoting tumor cell extravasation. Finally, L-Selectin (CD62L), constitutively expressed on leukocytes, regulates tumor-leukocyte interactions and promotes cell adhesion and hematogenous metastasis by favoring emboli formation. Further details regarding selectins may be found, e.g., in Cagnoni et al. (2016) Front Oncol. 6:109; Barthel et al. (2007) Expert Opin Ther Targets 11(11):1473-91; and Chen & Geng (2006) Arch Immunol Ther Exp 54(2):75-84; the disclosures of which are incorporated herein by reference in their entireties for all purposes. According to some embodiments, the glycan-binding moiety comprises the glycan-binding domain of a selectin selected from P-Selectin (CD62P), E-Selectin (CD62E), and L-Selectin (CD62L).

According to some embodiments, the glycan-binding moiety comprises the glycan-binding domain of a galectin. Galectins are a family of highly conserved glycan-binding soluble lectins, are defined by a conserved carbohydrate recognition domain (CRD) and a common structural fold. Vasta G R (2012) Adv Exp Med Biol 946:21-36. Based on structural features, mammalian galectins have been classified into three types: prototype galectins (Gal-1, -2, -5, -7, -10, -11, -13, -14, and -15, containing one CRD and existing as monomers or dimerizing through non-covalent interactions), tandem repeat-type galectins (Gal-4, -6, -8, -9, and -12), which exist as bivalent galectins containing two different CRDs connected by a linker peptide, and finally, Gal-3, the only chimera-type member of the galectin family. Galectins modulate different events in tumorigenesis and metastasis. Galectins contribute to immune tolerance and escape through apoptosis of effector T cells, regulation of clonal expansion, function of regulatory T cells (Tregs), and control of cytokine secretion. Expression levels for some galectins also change during malignant transformation, confirming their roles in cancer progression. Gal-1, abundantly secreted by almost all malignant tumor cells, has been characterized as a major promoter of an immunosuppressive protumorigenic microenvironment. Gal-3, another member of the family, has shown prominent protumorigenic effects in a multiplicity of tumors. Similar to Gal-1, Gal-3 signaling contributes to tilt the balance toward immunosuppressive TMEs by interacting with specific glycans, and impairing anti-tumor responses. In this regard, Gal-3 has been shown to promote anergy of tumor infiltrating lymphocytes (TILs). According to some embodiments, the glycan-binding moiety comprises the glycan-binding domain of a galectin selected from Gal-1, Gal-2, Gal-3, Gal-4, Gal-5, Gal-6, Gal-7, Gal-8, Gal-9, Gal-10, Gal-11, Gal-12, Gal-13, Gal-14, and Gal-15. In certain embodiments, the glycan-binding moiety comprises the glycan-binding domain of Gal-1. According to some embodiments, the glycan-binding moiety comprises the glycan-binding domain of Gal-3.

By “glycan-binding domain” or “sialoglycan-binding domain” of a lectin is meant the domain of a lectin or a glycan/sialoglycan-binding variant (e.g., glycan/sialoglycan-binding fragment) thereof responsible for binding to the respective glycan(s). Siglecs, for example, comprise an extracellular N-terminal V-set Ig (Ig-V) domain responsible for the binding of sialoside ligands. In certain embodiments, a “variant” of any of the polypeptides or domains thereof of the present disclosure contains one or more amino acid substitutions. According to some embodiments, the one or more amino acid substitutions are conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides contemplated in particular embodiments, polypeptides include polypeptides having at least about and still obtain a functional molecule that encodes a variant or derivative polypeptide with desirable characteristics. When it is desired to alter the amino acid sequence of a polypeptide to create an equivalent, or even an improved, variant polypeptide, one skilled in the art, for example, can change one or more of the codons of the encoding DNA sequence.

In addition to the glycan-binding domain of the lectin, the glycan-binding moiety may comprise one or more additional domains or variants (e.g., fragments) thereof of the lectin. By way of example, in the context of a Siglec, in addition to the sialoglycan-binding domain of the Siglec (e.g., Siglec-7, Siglec-9, Siglec-10, Siglec-15, etc.), the glycan-binding moiety may further comprise one or more (e.g., 1, 2, 3, or more) Ig-like domains or fragments thereof of the Siglec. The amino acid sequences and domains (e.g., extracellular domains) of Siglecs and other lectins are known, and any such domains may be included in the glycan-binding moiety as desired and/or useful.

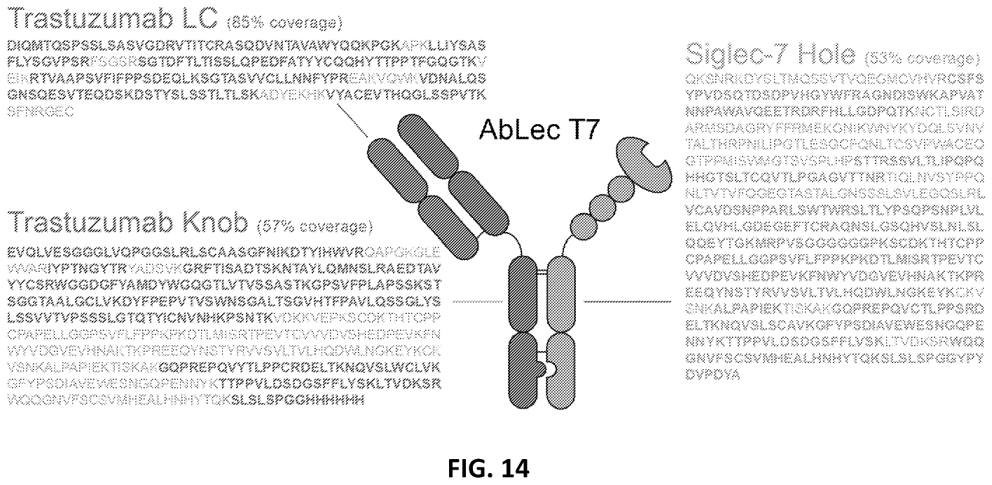

In certain embodiments, the glycan-binding moiety comprises an antibody heavy chain comprising a γ, α, δ, ε, or μ antibody heavy chain or fragment thereof. According to some embodiments, the antibody heavy chain or fragment thereof is an IgG heavy chain or fragment thereof, e.g., a human IgG1 heavy chain or fragment thereof. In certain embodiments, the antibody heavy chain or fragment thereof comprises a heavy chain constant region or fragment thereof. For example, when a heavy chain constant region or fragment thereof is included in the glycan-binding moiety, the antibody heavy chain constant region or fragment thereof may include one or more of a C_{H}1 domain, C_{H}2 domain, and/or C_{H}3 domain, e.g., a C_{H}2 domain and a C_{H}3 domain. According to some embodiments, the glycan-binding moiety comprises a C_{H}1 domain, a C_{H}2 domain, and a C_{H}3 domain. According to some embodiments, the glycan-binding moiety comprises a fragment crystallizable (Fc) region. According to some embodiments, each of the cell-targeting moiety and glycan-binding moiety comprises a C_{H}2 domain and/or a C_{H}3 domain (e.g., an Fc region), and the bispecific molecule is a heterodimer comprising knobs-into-holes modified domains, e.g., modified C_{H}3 domains. Non-limiting examples of such bispecific molecules of the present disclosure are schematically illustrated (with amino acid sequences) in FIGS. 14-17.

The “knob-in-hole” strategy (a non-limiting example of which is described, e.g., in WO 2006/028936) may be used to facilitate heterodimerization of the moieties of the bispecific molecules. Briefly, selected amino acids forming the interface of the CH3 domains in human IgG can be mutated at positions affecting CH3 domain interactions to promote heterodimer formation. An amino acid with a small side chain (hole) is introduced into a heavy chain of a moiety specifically binding a first target and an amino acid with a large side chain (knob) is introduced into a heavy chain of a moiety specifically binding a second target. After co-expression of the two moieties, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y7F405A, T366W/F405W, F405W/Y407A, T394W/Y407T, T3945/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface may be used, as described in US2010/0015133; US2009/0182127; US2010/028637 or US2011/0123532. In other strategies. heterodimerization may be promoted by the following substitutions (expressed as modified position in the first CH3 domain of the first heavy chain/modified position in the second CH3 domain of the second heavy chain): L351 Y_F405A_Y407V T394W, T3661_K392M_T394W/F405A_Y407V, T366L_K392M_T394W/F405A_Y407V, L351 Y_Y407A′T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849.

According to some embodiments, a bispecific molecule of the present disclosure is a fusion protein comprising the cell-targeting moiety fused to the glycan-binding moiety. When the bispecific molecule is a fusion protein, the cell-targeting moiety may be fused directly to the glycan-binding moiety (e.g., at the N- or C-terminus of the glycan binding moiety), or the cell-targeting moiety may be fused indirectly to the glycan-binding moiety via a linker. Any useful linkers may be employed, including but not limited to, a serine-glycine linker, or the like. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long. Also provided are nucleic acids that encode the fusion proteins of the present disclosure, as well as expression vectors comprising such nucleic acids, and host cells comprising such nucleic acids and/or expression vectors.

In certain embodiments, a bispecific molecule of the present disclosure is a conjugate comprising the cell-targeting moiety conjugated to the glycan-binding moiety via a linker. Non-limiting examples of linkers that may be employed in the conjugates of the present disclosure include ester linkers, amide linkers, maleimide or maleimide-based linkers; valine-citrulline linkers; hydrazone linkers; N-succinimidyl-4-(2-pyridyldithio)butyrate (SPDB) linkers; Succinimidyl-4-(N-maleimidomethyl)cyclohexane-1-carboxylate (SMCC) linkers; vinylsulfone-based linkers; linkers that include polyethylene glycol (PEG), such as, but not limited to tetraethylene glycol; linkers that include propanoic acid; linkers that include caproleic acid, and linkers including any combination thereof. In certain aspects, the linker is a chemically-labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell (e.g., a cancer cell). Chemically-labile linkers include, but are not limited to, hydrazone-based linkers, oxime-based linkers, carbonate-based linkers, ester-based linkers, etc. According to certain embodiments, the linker is an enzyme-labile linker, such as an enzyme-labile linker that is stable in the bloodstream but undergoes enzymatic cleavage upon internalization into a target cell, e.g., by a lysosomal protease (such as cathepsin or plasmin) in a lysosome of the target cell (e.g., a cancer cell). Enzyme-labile linkers include, but are not limited to, linkers that include peptidic bonds, e.g., dipeptide-based linkers such as valine-citrulline linkers, such as a maleimidocaproyl-valine-citruline-p-aminobenzyl (MC-vc-PAB) linker, a valyl-alanyl-para-aminobenzyloxy (Val-Ala-PAB) linker, and the like. Chemically-labile linkers, enzyme-labile, and non-cleavable linkers are known and described in detail, e.g., in Ducry & Stump (2010) Bioconjugate Chem. 21:5-13.

In certain embodiments, a conjugate of the present disclosure includes a linker that includes a valine-citrulline dipeptide, a valine-alanine dipeptide, or both. According to some embodiments, the linker is a valine-citruline-paraaminobenzyloxy (Val-Cit-PAB) linker. In certain embodiments, the linker is a valylalanylparaaminobenzyloxy (Val-Ala-PAB) linker.

The cell-targeting moiety may be conjugated to the glycan-binding moiety using any convenient approach. For example, the conjugating may include site-specifically conjugating the glycan-binding moiety to a pre-selected amino acid of the cell-targeting moiety (or vice versa). In certain aspects, the pre-selected amino acid is at the N-terminus or C-terminus of the cell-targeting moiety. In other aspects, the pre-selected amino acid is internal to the cell-targeting moiety—that is, between the N-terminal and C-terminal amino acid of the cell-targeting moiety. In some embodiments, the pre-selected amino acid is a non-natural amino acid. Non-limiting examples of non-natural amino acids which may be provided to the cell-targeting moiety (or glycan-binding moiety) to facilitate conjugation include those having a functional group selected from an azide, alkyne, alkene, amino-oxy, hydrazine, aldehyde (e.g., formylglycine, e.g., SMARTag™ technology from Catalent Pharma Solutions), nitrone, nitrile oxide, cyclopropene, norbornene, iso-cyanide, aryl halide, and boronic acid functional group. Unnatural amino acids which may be incorporated and selected to provide a functional group of interest are known and described in, e.g., Maza et al. (2015) Bioconjug. Chem. 26(9):1884-9; Patterson et al. (2014) ACS Chem. Biol. 9:592-605; Adumeau et al. (2016) Mol. Imaging Biol. (2):153-65; and elsewhere.

Numerous strategies are available for conjugating the cell-targeting moiety and glycan-binding moiety through a linker. For example, the glycan-binding moiety may be derivatized by covalently attaching the linker to the glycan-binding moiety, where the linker has a functional group capable of reacting with a “chemical handle” on the cell-targeting moiety. Also by way of example, the cell-targeting moiety may be derivatized by covalently attaching the linker to the cell-targeting moiety, where the linker has a functional group capable of reacting with a “chemical handle” on the glycan-binding moiety. The functional group on the linker may vary and may be selected based on compatibility with the chemical handle on the cell-targeting moiety or glycan-binding moiety. According to one embodiment, the chemical handle is provided by incorporation of an unnatural amino acid having the chemical handle into the cell-targeting moiety or glycan-binding moiety. In some embodiments, conjugating the cell-targeting moiety and glycan-binding moiety is by copper-free, strain-promoted cycloaddition, alkyne-azide cycloaddition, or the like.

Using the information provided herein, the cell-targeting and glycan-binding moieties and fusion proteins of the present disclosure may be prepared using standard techniques known to those of skill in the art. For example, a nucleic acid sequence(s) encoding the amino acid sequences of the cell-targeting and glycan-binding moieties of the bispecific molecules of the present disclosure can be used to express the cell-targeting and glycan-binding moieties. The nucleic acid sequence(s) can be optimized to reflect particular codon “preferences” for various expression systems according to standard methods known to those of skill in the art. Using the sequence information provided, the nucleic acids may be synthesized according to a number of standard methods known to those of skill in the art.

Once a nucleic acid(s) encoding a subject cell-targeting and/or glycan-binding moiety is synthesized, it can be amplified and/or cloned according to standard methods. Molecular cloning techniques to achieve these ends are known in the art. A wide variety of cloning and in vitro amplification methods suitable for the construction of recombinant nucleic acids are known to persons of skill in the art and are the subjects of numerous textbooks and laboratory manuals.

Expression of natural or synthetic nucleic acids encoding the cell-targeting and/or glycan-binding moieties of the present disclosure can be achieved by operably linking a nucleic acid encoding the cell-targeting and/or glycan-binding moieties to a promoter (which is either constitutive or inducible), and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the cell-targeting and/or glycan-binding moieties. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid it is common to construct expression plasmids which typically contain a strong promoter to direct transcription, a ribosome binding site for translational initiation, and a transcription/translation terminator, each in functional orientation to each other and to the protein-encoding sequence. Examples of regulatory regions suitable for this purpose in E. coli are the promoter and operator region of the E. coli tryptophan biosynthetic pathway, the leftward promoter of phage lambda (P_{L}), and the L-arabinose (araBAD) operon. The inclusion of selection markers in DNA vectors transformed in E. coli is also useful. Examples of such markers include genes specifying resistance to ampicillin, tetracycline, or chloramphenicol. Expression systems for expressing antibodies are available using, for example, E. coli, Bacillus sp. and Salmonella. E. coli systems may also be used.

The cell-targeting and/or glycan-binding moiety gene(s) may also be subcloned into an expression vector that allows for the addition of a tag (e.g., FLAG, hexahistidine, and the like) at the C-terminal end or the N-terminal end of the cell-targeting and/or glycan-binding moiety to facilitate purification. Methods of transfecting and expressing genes in mammalian cells are known in the art. Transducing cells with nucleic acids can involve, for example, incubating lipidic microparticles containing nucleic acids with cells or incubating viral vectors containing nucleic acids with cells within the host range of the vector. The culture of cells used in the present disclosure, including cell lines and cultured cells from tissue (e.g., tumor) or blood samples is known in the art.

Once the nucleic acid encoding a subject cell-targeting and/or glycan-binding moiety is isolated and cloned, one can express the nucleic acid in a variety of recombinantly engineered cells known to those of skill in the art. Examples of such cells include bacteria, yeast, filamentous fungi, insect (e.g. those employing baculoviral vectors), and mammalian cells.

Isolation and purification of a subject cell-targeting and/or glycan-binding moiety can be accomplished according to methods known in the art. For example, a protein can be isolated from a lysate of cells genetically modified to express the protein constitutively and/or upon induction, or from a synthetic reaction mixture, by immunoaffinity purification (or precipitation using Protein L or A), washing to remove non-specifically bound material, and eluting the specifically bound cell-targeting and/or glycan-binding moiety. The isolated cell-targeting and/or glycan-binding moiety can be further purified by dialysis and other methods normally employed in protein purification methods. In one embodiment, the cell-targeting and/or glycan-binding moiety may be isolated using metal chelate chromatography methods. Cell-targeting and/or glycan-binding moieties of the present disclosure may contain modifications to facilitate isolation, as discussed above.

The cell-targeting and/or glycan-binding moieties may be prepared in substantially pure or isolated form (e.g., free from other polypeptides). The protein can be present in a composition that is enriched for the polypeptide relative to other components that may be present (e.g., other polypeptides or other host cell components). Purified cell-targeting and/or glycan-binding moieties may be provided such that the cell-targeting and/or glycan-binding moiety is present in a composition that is substantially free of other expressed proteins, e.g., less than 90%, usually less than 60% and more usually less than 50% of the composition is made up of other expressed proteins.

The cell-targeting and/or glycan-binding moieties produced by prokaryotic cells may require exposure to chaotropic agents for proper folding. During purification from E. coli, for example, the expressed protein can be optionally denatured and then renatured. This can be accomplished, e.g., by solubilizing the bacterially produced cell-targeting and/or glycan-binding moieties in a chaotropic agent such as guanidine HCl. The cell-targeting and/or glycan-binding moiety is then renatured, either by slow dialysis or by gel filtration. Alternatively, nucleic acid encoding the cell-targeting and/or glycan-binding moieties may be operably linked to a secretion signal sequence such as pelB so that the cell-targeting and/or glycan-binding moieties are secreted into the periplasm in correctly-folded form.

### Nucleic Acids, Expression Vectors and Cells

In view of the section above regarding methods of producing the cell-targeting and/or glycan-binding moieties of the bispecific molecules of the present disclosure, it will be appreciated that the present disclosure also provides nucleic acids, expression vectors and cells.

In certain embodiments, provided is a nucleic acid encoding any of the cell-targeting moieties of the bispecific molecules of the present disclosure, any of the glycan-binding moieties of the bispecific molecules of the present disclosure, or both. Non-limiting examples of nucleotide sequences encoding cell-targeting and glycan-binding moieties of bispecific molecules according to embodiments of the present disclosure are provided in the Experimental section below.

Also provided are expression vectors comprising any of the nucleic acids of the present disclosure. Expression of natural or synthetic nucleic acids encoding the cell-targeting and/or glycan-binding moieties can be achieved by operably linking a nucleic acid encoding the cell-targeting and/or glycan-binding moieties to a promoter (which is either constitutive or inducible) and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the cell-targeting and/or glycan-binding moieties. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

Cells that comprise any of the nucleic acids and/or expression vectors of the present disclosure are also provided. According to some embodiments, a cell of the present disclosure comprises a nucleic acid that encodes any of the cell-targeting moieties of the bispecific molecules of the present disclosure, any of the glycan-binding moieties of the bispecific molecules of the present disclosure, or both. In certain such embodiments, the bispecific molecule is a fusion protein (as described above) and the nucleic acid encodes the fusion protein. According to some embodiments, provided is a cell comprising a first nucleic acid encoding any of the cell-targeting moieties of the bispecific molecules of the present disclosure, and a second nucleic acid encoding any of the glycan-binding moieties of the bispecific molecules of the present disclosure. In certain embodiments, such as cell comprises a first expression vector comprising the first nucleic acid, and a second expression vector comprising the second nucleic acid.

Also provided are methods of making the bispecific molecule of the present disclosure, the methods comprising culturing a cell of the present disclosure under conditions suitable for the cell to express the cell-targeting moiety and/or the glycan-binding moiety, wherein the cell-targeting moiety and/or the glycan-binding moiety is produced. The conditions for culturing the cell such that the cell-targeting moiety and/or the glycan-binding moiety is expressed may vary. Such conditions may include culturing the cell in a suitable container (e.g., a cell culture plate or well thereof), in suitable medium (e.g., cell culture medium, such as DMEM, RPMI, MEM, IMDM, DMEM/F-12, or the like) at a suitable temperature (e.g., 32° C.-42° C., such as 37° C.) and pH (e.g., pH 7.0-7.7, such as pH 7.4) in an environment having a suitable percentage of CO₂, e.g., 3% to 10%, such as 5%).

### Additional Bispecific Molecules

Also provided by the present disclosure are bispecific molecules as described elsewhere herein, but where the second moiety binds to a target other than a glycan. That is, with the benefit of the present disclosure, it will be understood that the AbLecs of the present disclosure provide proof of concept that the technology may be applied to contexts beyond the glycan-binding context. In certain embodiments, provided are bispecific molecules that comprise a cell-targeting moiety (e.g., any of the cell-targeting moieties described elsewhere herein) fused to an Fc region, and a moiety comprising a ligand-binding domain of a receptor, where the moiety comprising a ligand-binding domain of a receptor is also fused to an Fc region. In certain embodiments, the two moieties are heterodimerized via the Fc regions. In some embodiments, heterodimerization via the Fc regions is via a knobs-in-holes strategy as described elsewhere herein.

In some embodiments, the moiety comprising a ligand-binding domain of a receptor comprises the ligand-binding domain of a receptor that binds to a cell surface ligand. That is, the ligand-binding domain binds to a cell surface ligand. In certain embodiments, the cell surface ligand is present on the surface of a cell that also displays the target for the cell targeting moiety, such that the cell-targeting moiety and second moiety (the moiety comprising a ligand-binding domain of a receptor) bind to different types of molecules present on the surface of the same cell.

In certain embodiments, the moiety comprising a ligand-binding domain of a receptor comprises the ligand-binding domain of a stem cell receptor, immune cell receptor, growth factor receptor, cytokine receptor, hormone receptor, receptor tyrosine kinase, a receptor in the epidermal growth factor receptor (EGFR) family (e.g., HER2 (human epidermal growth factor receptor 2), etc.), a receptor in the fibroblast growth factor receptor (FGFR) family, a receptor in the vascular endothelial growth factor receptor (VEGFR) family, a receptor in the platelet derived growth factor receptor (PDGFR) family, a receptor in the rearranged during transfection (RET) receptor family, a receptor in the Eph receptor family, a receptor in the discoidin domain receptor (DDR) family, and a mucin protein (e.g., MUC1). In some embodiments, the moiety comprising a ligand-binding domain of a receptor comprises the ligand-binding domain of CD71 (transferrin receptor). In certain embodiments, the moiety comprising a ligand-binding domain of a receptor comprises the ligand-binding domain of an immune cell receptor, non-limiting examples of which include a T cell receptor, a B cell receptor, a natural killer (NK) cell receptor, a macrophage receptor, a monocyte receptor, a neutrophil receptor, a dendritic cell receptor, a mast cell receptor, a basophil receptor, and an eosinophil receptor.

### Compositions

Aspects of the present disclosure further include compositions. According to some embodiments, a composition of the present disclosure includes a bispecific molecule of the present disclosure. For example, the bispecific molecule may be any of the bispecific molecules described in the Bispecific Molecule section hereinabove, which descriptions are incorporated but not reiterated herein for purposes of brevity.

In certain aspects, a composition of the present disclosure includes the bispecific molecule present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid salt sodium (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

Aspects of the present disclosure further include pharmaceutical compositions. In some embodiments, a pharmaceutical composition of the present disclosure comprises a bispecific molecule of the present disclosure, and a pharmaceutically acceptable carrier.

The bispecific molecules can be incorporated into a variety of formulations for therapeutic administration. More particularly, the bispecific molecules can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the bispecific molecules for administration to an individual (e.g., suitable for human administration) are generally sterile and may further be free of detectable pyrogens or other contaminants contraindicated for administration to a patient according to a selected route of administration.

In pharmaceutical dosage forms, the bispecific molecules can be administered in the form of their pharmaceutically acceptable salts, or they may also be used alone or in appropriate association, as well as in combination, with other pharmaceutically active compounds. The following methods and carriers/excipients are merely examples and are in no way limiting.

For oral preparations, the bispecific molecules can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The bispecific molecules can be formulated for parenteral (e.g., intravenous, intra-arterial, intraosseous, intramuscular, intracerebral, intracerebroventricular, intrathecal, subcutaneous, etc.) administration. In certain aspects, the bispecific molecules are formulated for injection by dissolving, suspending or emulsifying the bispecific molecules in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the bispecific molecules may be prepared by mixing the bispecific molecules having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methyIglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the bispecific molecules may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the bispecific molecule against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included, e.g., in an amount of about 10 mM to 500 nM.

In some embodiments, the pharmaceutical composition includes the bispecific molecule, and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Kits

Aspects of the present disclosure further include kits. In certain embodiments, the kits find use in practicing the methods of the present disclosure, e.g., methods comprising administering a pharmaceutical composition of the present disclosure to an individual to enhance anti-tumor immunity in the individual, administering a pharmaceutical composition of the present disclosure to an individual to enhance or suppress an immune response in an individual, or the like.

Accordingly, in certain embodiments, a kit of the present disclosure comprises one or more unit dosages of a pharmaceutical composition of the present disclosure, and instructions for administering the pharmaceutical composition to an individual in need thereof. The pharmaceutical composition included in the kit may include any of the bispecific molecules of the present disclosure, e.g., any of the bispecific molecules described hereinabove, which are not reiterated herein for purposes of brevity.

The kits of the present disclosure may include a quantity of the compositions, present in unit dosages, e.g., ampoules, or a multi-dosage format. As such, in certain embodiments, the kits may include one or more (e.g., two or more) unit dosages (e.g., ampoules) of a composition that includes bispecific molecule of the present disclosure. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular bispecific molecule employed, the effect to be achieved, and the pharmacodynamics associated with the bispecific molecule, in the individual. In yet other embodiments, the kits may include a single multi dosage amount of the composition.

In certain embodiments, a kit of the present disclosure includes instructions for administering the one or more unit dosages of the pharmaceutical composition to an individual in need of enhancement of anti-tumor immunity. According to some embodiments, a kit of the present disclosure includes instructions for administering the one or more unit dosages of the pharmaceutical composition to an individual in need of enhancement or suppression of an immune response.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

### Methods

Aspects of the present disclosure include methods of using the bispecific molecules of the present disclosure. The methods are useful in a variety of contexts, including in vitro and/or in vivo research and/or clinical applications.

In certain aspects, provided are methods of enhancing anti-tumor immunity in an individual in need thereof. Such methods comprise administering an effective amount of a pharmaceutical composition of the present disclosure to the individual, e.g., a pharmaceutical composition comprising a bispecific molecule of the present disclosure comprising a cancer cell-targeting moiety and a glycan-binding moiety. In certain embodiments, the methods are for enhancing antibody-dependent cellular phagocytosis (ADCP) and/or cytotoxicity (ADCC) in the individual.

In certain aspects, provided are methods of enhancing or suppressing an immune response in an individual in need thereof. Such methods comprise administering an effective amount of a pharmaceutical composition of the present disclosure to the individual, e.g., a pharmaceutical composition comprising a bispecific molecule of the present disclosure comprising an immune cell-targeting moiety and a glycan-binding moiety.

The individual in need thereof may have a cell proliferative disorder. By “cell proliferative disorder” is meant a disorder wherein unwanted cell proliferation of one or more subset(s) of cells in a multicellular organism occurs, resulting in harm, for example, pain or decreased life expectancy to the organism. Cell proliferative disorders include, but are not limited to, cancer, pre-cancer, benign tumors, blood vessel proliferative disorders (e.g., arthritis, restenosis, and the like), fibrotic disorders (e.g., hepatic cirrhosis, atherosclerosis, and the like), psoriasis, epidermic and dermoid cysts, lipomas, adenomas, capillary and cutaneous hemangiomas, lymphangiomas, nevi lesions, teratomas, nephromas, myofibromatosis, osteoplastic tumors, dysplastic masses, mesangial cell proliferative disorders, and the like.

In some embodiments, the individual has cancer. The subject methods may be employed for the treatment of a large variety of cancers. “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancers that may be treated using the subject methods include, but are not limited to, carcinoma, lymphoma, blastoma, and sarcoma. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bile duct cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, various types of head and neck cancer, and the like. In certain embodiments, the individual has a cancer selected from a solid tumor, recurrent glioblastoma multiforme (GBM), non-small cell lung cancer, metastatic melanoma, melanoma, peritoneal cancer, epithelial ovarian cancer, glioblastoma multiforme (GBM), metastatic colorectal cancer, colorectal cancer, pancreatic ductal adenocarcinoma, squamous cell carcinoma, esophageal cancer, gastric cancer, neuroblastoma, fallopian tube cancer, bladder cancer, metastatic breast cancer, pancreatic cancer, soft tissue sarcoma, recurrent head and neck cancer squamous cell carcinoma, head and neck cancer, anaplastic astrocytoma, malignant pleural mesothelioma, breast cancer, squamous non-small cell lung cancer, rhabdomyosarcoma, metastatic renal cell carcinoma, basal cell carcinoma (basal cell epithelioma), and gliosarcoma. In certain aspects, the individual has a cancer selected from melanoma, Hodgkin lymphoma, renal cell carcinoma (RCC), bladder cancer, non-small cell lung cancer (NSCLC), and head and neck squamous cell carcinoma (HNSCC).

The bispecific molecules of the present disclosure may be administered via a route of administration selected from oral (e.g., in tablet form, capsule form, liquid form, or the like), parenteral (e.g., by intravenous, intra-arterial, subcutaneous, intramuscular, or epidural injection), topical, intra-nasal, or intra-tumoral administration.

The bispecific molecules of the present disclosure may be administered in a pharmaceutical composition in a therapeutically effective amount. By “therapeutically effective amount” is meant a dosage sufficient to produce a desired result, e.g., an amount sufficient to effect beneficial or desired therapeutic (including preventative) results, such as a reduction in a symptom of a cancer and/or immune disorder, as compared to a control. With respect to cancer, in some embodiments, the therapeutically effective amount is sufficient to slow the growth of a tumor, reduce the size of a tumor, and/or the like. An effective amount can be administered in one or more administrations.

Aspects of the present disclosure include methods for treating a cancer and/or immune disorder of an individual. By treatment is meant at least an amelioration of one or more symptoms associated with the cancer and/or immune disorder of the individual, where amelioration is used in a broad sense to refer to at least a reduction in the magnitude of a parameter, e.g., symptom, associated with the cancer and/or immune disorder being treated. As such, treatment also includes situations where the cancer and/or immune disorder, or at least one or more symptoms associated therewith, are completely inhibited, e.g., prevented from happening, or stopped, e.g., terminated, such that the individual no longer suffers from the cancer and/or immune disorder, or at least the symptoms that characterize the cancer and/or immune disorder.

A bispecific molecule of the present disclosure may be administered to the individual alone or in combination with a second agent. Second agents of interest include, but are not limited to, agents approved by the United States Food and Drug Administration and/or the European Medicines Agency (EMA) for use in treating cancer. In some embodiments, the second agent is an immune checkpoint inhibitor. Immune checkpoint inhibitors of interest include, but are not limited to, a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, a T cell immunoreceptor with Ig and ITIM domains (TIGIT) inhibitor, a V-domain Ig suppressor of T cell activation (VISTA) inhibitor, a B7-H3 inhibitor, and any combination thereof.

When a bispecific molecule of the present disclosure is administered with a second agent, the bispecific molecule and the second agent may be administered to the individual according to any suitable administration regimen. According to certain embodiments, the bispecific molecule and the second agent are administered according to a dosing regimen approved for individual use. In some embodiments, the administration of the bispecific molecule permits the second agent to be administered according to a dosing regimen that involves one or more lower and/or less frequent doses, and/or a reduced number of cycles as compared with that utilized when the second agent is administered without administration of the bispecific molecule. In certain aspects, the administration of the second agent permits the bispecific molecule to be administered according to a dosing regimen that involves one or more lower and/or less frequent doses, and/or a reduced number of cycles as compared with that utilized when the bispecific molecule is administered without administration of the second agent.

In some embodiments, one or more doses of the bispecific molecule and the second agent are administered concurrently to the individual. By “concurrently” is meant the bispecific molecule and the second agent are either present in the same pharmaceutical composition, or the bispecific molecule and the second agent are administered as separate pharmaceutical compositions within 1 hour or less, 30 minutes or less, or 15 minutes or less.

In some embodiments, one or more doses of the bispecific molecule and the second agent are administered sequentially to the individual.

In some embodiments, the bispecific molecule and the second agent are administered to the individual in different compositions and/or at different times. For example, the bispecific molecule may be administered prior to administration of the second agent, e.g., in a particular cycle. Alternatively, the second agent may be administered prior to administration of the bispecific molecule, e.g., in a particular cycle. The second agent to be administered may be administered a period of time that starts at least 1 hour, 3 hours, 6 hours, 12 hours, 24 hours, 48 hours, 72 hours, or up to 5 days or more after the administration of the first agent to be administered.

In one example, the second agent is administered to the individual for a desirable period of time prior to administration of the bispecific molecule. In certain aspects, when the individual has cancer, such a regimen “primes” the cancer cells to potentiate the anti-cancer effect of the bispecific molecule. Such a period of time separating a step of administering the second agent from a step of administering the bispecific molecule is of sufficient length to permit priming of the cancer cells, desirably so that the anti-cancer effect of the bispecific molecule is increased.

In some embodiments, administration of one agent is specifically timed relative to administration of the other agent. For example, in some embodiments, the bispecific molecule is administered so that a particular effect is observed (or expected to be observed, for example based on population studies showing a correlation between a given dosing regimen and the particular effect of interest).

In certain aspects, desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular individual of interest.

In some embodiments, the bispecific molecule and the second agent are administered according to an intermittent dosing regimen including at least two cycles. Where two or more agents are administered in combination, and each by such an intermittent, cycling, regimen, individual doses of different agents may be interdigitated with one another. In certain aspects, one or more doses of a second agent is administered a period of time after a dose of the first agent. In some embodiments, each dose of the second agent is administered a period of time after a dose of the first agent. In certain aspects, each dose of the first agent is followed after a period of time by a dose of the second agent. In some embodiments, two or more doses of the first agent are administered between at least one pair of doses of the second agent; in certain aspects, two or more doses of the second agent are administered between at least one pair of doses of the first agent. In some embodiments, different doses of the same agent are separated by a common interval of time; in some embodiments, the interval of time between different doses of the same agent varies. In certain aspects, different doses of the bispecific molecule and the second agent are separated from one another by a common interval of time; in some embodiments, different doses of the different agents are separated from one another by different intervals of time.

One exemplary protocol for interdigitating two intermittent, cycled dosing regimens may include: (a) a first dosing period during which a therapeutically effective amount the bispecific molecule is administered to the individual; (b) a first resting period; (c) a second dosing period during which a therapeutically effective amount of the second agent is administered to the individual; and (d) a second resting period. A second exemplary protocol for interdigitating two intermittent, cycled dosing regimens may include: (a) a first dosing period during which a therapeutically effective amount the second agent is administered to the individual; (b) a first resting period; (c) a second dosing period during which a therapeutically effective amount of the bispecific molecule is administered to the individual; and (d) a second resting period.

In some embodiments, the first resting period and second resting period may correspond to an identical number of hours or days. Alternatively, in some embodiments, the first resting period and second resting period are different, with either the first resting period being longer than the second one or, vice versa. In some embodiments, each of the resting periods corresponds to 120 hours, 96 hours, 72 hours, 48 hours, 24 hours, 12 hours, 6 hours, 30 hours, 1 hour, or less. In some embodiments, if the second resting period is longer than the first resting period, it can be defined as a number of days or weeks rather than hours (for instance 1 day, 3 days, 5 days, 1 week, 2, weeks, 4 weeks or more).

If the first resting period's length is determined by existence or development of a particular biological or therapeutic event, then the second resting period's length may be determined on the basis of different factors, separately or in combination. Exemplary such factors may include type and/or stage of a cancer against which the therapy is administered; properties (e.g., pharmacokinetic properties) of the bispecific molecule, and/or one or more features of the patient's response to therapy with the bispecific molecule. In some embodiments, length of one or both resting periods may be adjusted in light of pharmacokinetic properties (e.g., as assessed via plasma concentration levels) of one or the other of the administered agents. For example, a relevant resting period might be deemed to be completed when plasma concentration of the relevant agent is below a pre-determined level, optionally upon evaluation or other consideration of one or more features of the individual's response.

In certain aspects, the number of cycles for which a particular agent is administered may be determined empirically. Also, in some embodiments, the precise regimen followed (e.g., number of doses, spacing of doses (e.g., relative to each other or to another event such as administration of another therapy), amount of doses, etc.) may be different for one or more cycles as compared with one or more other cycles.

The bispecific molecule and the second agent may be administered together or independently via any suitable route of administration. The bispecific molecule and the second agent may be administered via a route of administration independently selected from oral, parenteral (e.g., by intravenous, intra-arterial, subcutaneous, intramuscular, or epidural injection), topical, or intra-nasal administration. According to certain embodiments, the bispecific molecule and the second agent are both administered orally (e.g., in tablet form, capsule form, liquid form, or the like) either concurrently (in the same pharmaceutical composition or separate pharmaceutical compositions) or sequentially.

Notwithstanding the appended claims, the present disclosure is also defined by the following embodiments:

1. A bispecific molecule comprising:
a cell-targeting moiety; and
a glycan-binding moiety.
   2. The bispecific molecule of embodiment 1, wherein the cell-targeting moiety is a cancer cell-targeting moiety.
   3. The bispecific molecule of embodiment 2, wherein the cancer cell-targeting moiety binds to a cancer cell surface molecule selected from the group consisting of: 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD20, CD22, CD25, CD27L, CD30, CD33, CD37, CD44, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvIII, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), GD2 ganglioside, glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), programmed cell death receptor ligand 1 (PD-L1), programmed cell death receptor ligand 2 (PD-L2), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), Tn antigen, trophoblast cell-surface antigen (TROP-2), and Wilms' tumor 1 (WT1).
   4. The bispecific molecule of embodiment 1, wherein the cell-targeting moiety is an immune cell-targeting moiety.
   5. The bispecific molecule of embodiment 4, wherein the immune cell-targeting moiety binds to an immune cell surface molecule selected from the group consisting of: PD-1, PD-L1, PD-L2, CLTA-4, VISTA, LAG-3, TIM-3, CD24, CD47, SIRPalpha, CD3, CD8, CD4, CD28, CD80, CD86, CD19, ICOS, OX40, OX40L, GD3 ganglioside, TIGIT, Siglec-2, Siglec-3, Siglec-7, Siglec-8, Siglec-9, Siglec-10, Siglec-15, galectin-9, B7-H3, B7-H4, CD40, CD40L, B7RP1, CD70, CD27, BTLA, HVEM, KIR, 4-1BB, 4-1BBL, CD226, CD155, CD112, GITR, GITRL, A2aR, CD137, CD137L, CD45, CD206, CD163, TRAIL, NKG2D, CD16, and TGF-beta.
   6. The bispecific molecule of any one of embodiments 1 to 5, wherein the glycan-binding moiety comprises a sialoglycan-binding moiety.
   7. The bispecific molecule of embodiment 6, wherein the sialoglycan-binding moiety comprises the sialoglycan-binding domain of a lectin.
   8. The bispecific molecule of embodiment 7, wherein the lectin is a sialic acid-binding immunoglobulin-like lectin (Siglec).
   9. The bispecific molecule of embodiment 8, wherein the Siglec is a CD33-related Siglec.
   10. The bispecific molecule of embodiment 9, wherein the CD33-related Siglec is selected from the group consisting of: Siglec-7, Siglec-9, and Siglec-10.
   11. The bispecific molecule of embodiment 10, wherein the CD33-related Siglec is Siglec-7.
   12. The bispecific molecule of embodiment 10, wherein the CD33-related Siglec is Siglec-9.
   13. The bispecific molecule of embodiment 8, wherein the Siglec is Siglec-15.
   14. The bispecific molecule of embodiment 6, wherein the sialoglycan-binding moiety comprises the sialoglycan-binding domain of a Siglec-like adhesin.
   15. The bispecific molecule of any one of embodiments 1 to 5, wherein the glycan-binding moiety comprises the glycan-binding domain of a C-type lectin.
   16. The bispecific molecule of embodiment 15, wherein the C-type lectin is DECTIN-1, lectin-like oxidized low-density lipoprotein receptor-1 (LOX-1), C-type lectin-like receptor-1 (CLEC-1), C-type lectin-like receptor 2 (CLEC-2), myeloid inhibitory C-type lectin-like receptor (MICL), CLEC9A, DC immunoreceptor (DCIR), DECTIN-2, blood DC antigen-2 (BDCA-2), macrophage-inducible C-type lectin (MINCLE), macrophage galactose lectin (MGL), or asialoglycoprotein receptor (ASGPR).
   17. The bispecific molecule of any one of embodiments 1 to 5, wherein the glycan-binding moiety comprises the glycan-binding domain of a galectin.
   18. The bispecific molecule of embodiment 17, wherein the galectin is Gal-1, Gal-2, Gal-3, Gal-4, Gal-5, Gal-6, Gal-7, Gal-8, Gal-9, Gal-10, Gal-11, Gal-12, Gal-13, Gal-14, or Gal-15.
   19. The bispecific molecule of embodiment 17, wherein the galectin is Gal-1.
   20. The bispecific molecule of embodiment 17, wherein the galectin is Gal-3.
   21. The bispecific molecule of any one of embodiments 1 to 5, wherein the glycan-binding moiety comprises the glycan-binding domain of a selectin.
   22. The bispecific molecule of embodiment 21, wherein the selectin is P-Selectin (CD62P), E-Selectin (CD62E), or L-Selectin (CD62L).
   23. The bispecific molecule of any one of embodiments 1 to 22, wherein the cell-targeting moiety comprises a ligand for a receptor on the surface of a target cell, or a small molecule that binds to a cell surface molecule on a target cell.
   24. The bispecific molecule of any one of embodiments 1 to 22, wherein the cell-targeting moiety comprises the antigen-binding domain of an antibody.
   25. The bispecific molecule of any one of embodiments 1 to 22, wherein the cell-targeting moiety comprises an antibody heavy chain comprising a variable heavy chain (V_{H}) region and an antibody light chain comprising a variable light chain (V_{L}) region.
   26. The bispecific molecule of embodiment 25, wherein the antibody heavy chain comprises a CH1 domain, a hinge region, a CH2 domain, a CH3 domain, or any combination thereof.
   27. The bispecific molecule of embodiment 25 or embodiment 26, wherein the antibody heavy chain comprises a CH2 domain, a CH3 domain, or both.
   28. The bispecific molecule of any one of embodiments 25 to 27, wherein the antibody heavy chain comprises a fragment crystallizable (Fc) region.
   29. The bispecific molecule of any one of embodiments 1 to 28, wherein the glycan-binding moiety comprises an antibody heavy chain domain.
   30. The bispecific molecule of embodiment 29, wherein the antibody heavy chain domain of the glycan-binding moiety comprises a CH1 domain, a hinge region, a CH2 domain, a CH3 domain, or any combination thereof.
   31. The bispecific molecule of embodiment 29 or embodiment 30, wherein the antibody heavy chain domain of the glycan-binding moiety comprises a CH2 domain, a CH3 domain, or both.
   32. The bispecific molecule of any one of embodiments 29 to 31, wherein the antibody heavy chain domain of the glycan-binding moiety comprises a fragment crystallizable (Fc) region.
   33. The bispecific molecule of embodiment 31 or embodiment 32, wherein the cell-targeting moiety comprises an antibody heavy chain comprising a CH3 domain, and wherein the bispecific molecule is a heterodimer comprising knobs-into-holes modified CH3 domains.
   34. The bispecific molecule of any one of embodiments 1 to 32, wherein the bispecific molecule is a fusion protein comprising the cell-targeting moiety fused to the glycan-binding moiety.
   35. The bispecific molecule of embodiment 34, wherein the cell-targeting moiety is fused directly to the glycan-binding moiety.
   36. The bispecific molecule of embodiment 34, wherein the cell-targeting moiety is fused indirectly to the glycan-binding moiety via a linker.
   37. The bispecific molecule of any one of embodiments 1 to 32, wherein the bispecific molecule is a conjugate comprising the cell-targeting moiety conjugated to the glycan-binding moiety.
   38. A nucleic acid that encodes:
a cell-targeting moiety of the bispecific molecule of any one of embodiments 1 to 36;
a glycan-binding moiety of the bispecific molecule of any one of embodiments 1 to 36; or
both.
   39. An expression vector comprising the nucleic acid of embodiment 38.
   40. A pharmaceutical composition comprising:
the bispecific molecule of any one of embodiments 1 to 37; and
a pharmaceutically-acceptable carrier.
   41. The pharmaceutical composition of embodiment 40, wherein the cell-targeting moiety is a cancer cell-targeting moiety.
   42. The pharmaceutical composition of embodiment 40, wherein the cell-targeting moiety is an immune cell-targeting moiety.
   43. A kit comprising:
one or more unit dosages of the pharmaceutical composition of any one of embodiments 40 to 42; and
instructions for administering the one or more unit dosages of the pharmaceutical composition to an individual in need thereof.
   44. The kit of embodiment 43, comprising two or more unit dosages of the pharmaceutical composition.
   45. The kit of embodiment 43 or embodiment 44, wherein the cell-targeting moiety is a cancer cell-targeting moiety, and wherein the instructions comprise instructions for administering the one or more unit dosages of the pharmaceutical composition to an individual in need of enhancement of anti-tumor immunity.
   46. The kit of embodiment 43 or embodiment 44, wherein the cell-targeting moiety is an immune cell-targeting moiety, and wherein the instructions comprise instructions for administering the one or more unit dosages of the pharmaceutical composition to an individual in need of enhancement or suppression of an immune response.
   47. A method of enhancing anti-tumor immunity in an individual in need thereof, comprising: administering an effective amount of the pharmaceutical composition of embodiment 41 to the individual.
   48. A method of enhancing or suppressing an immune response in an individual in need thereof, comprising:

administering an effective amount of the pharmaceutical composition of embodiment 42 to the individual.

49. The method according to embodiment 47 or embodiment 48, wherein the administering is by parenteral administration.
50. A bispecific molecule comprising:
a cell-targeting moiety fused to an Fc region; and
a moiety comprising a ligand-binding domain of a receptor fused to an Fc region,
wherein the cell-targeting moiety and the moiety comprising a ligand-binding domain of a receptor are heterodimerized via the Fc regions.
   51. The bispecific molecule of embodiment 50, wherein the cell targeting moiety is as defined in any one of embodiments 2 to 5.
   52. The bispecific molecule of embodiment 50 or embodiment 51, wherein the moiety comprising a ligand-binding domain of a receptor comprises the ligand-binding domain of a receptor that binds to a cell surface ligand.
   53. The bispecific molecule of any one of embodiments 50 to 52, wherein the bispecific molecule is a heterodimer comprising knobs-into-holes modified CH3 domains.

The following examples are offered by way of illustration and not by way of limitation.

### EXPERIMENTAL

### Example 1—Enhancement of Anti-Tumor Immune Responses Using Bispecific Molecules Comprising Cancer Cell-Targeting Moieties and Glycan-Binding Moieties

Described herein are bispecific molecules comprising a cell-targeting moiety and a glycan-binding moiety, and the demonstration that such molecules are effective for enhancing anti-tumor immune responses, e.g., by enhanced antibody-dependent cellular phagocytosis (ADCP) and/or cytotoxicity (ADCC).

As proof-of-principle, described herein is a new class of antibody-lectin bispecific molecules (sometimes referred to herein as “AbLecs”) targeting tumor-associated sialoglycans for checkpoint blockade. In this approach, recombinant Siglec binding domains with sialoglycan binding specificity are coupled to high-affinity tumor-targeting antibody binding domains. The AbLecs are expected to accumulate with high effective molarity on the cancer cell surface, permitting otherwise low affinity recombinant Siglecs to bind cell surface sialoglycans at therapeutically relevant concentrations.

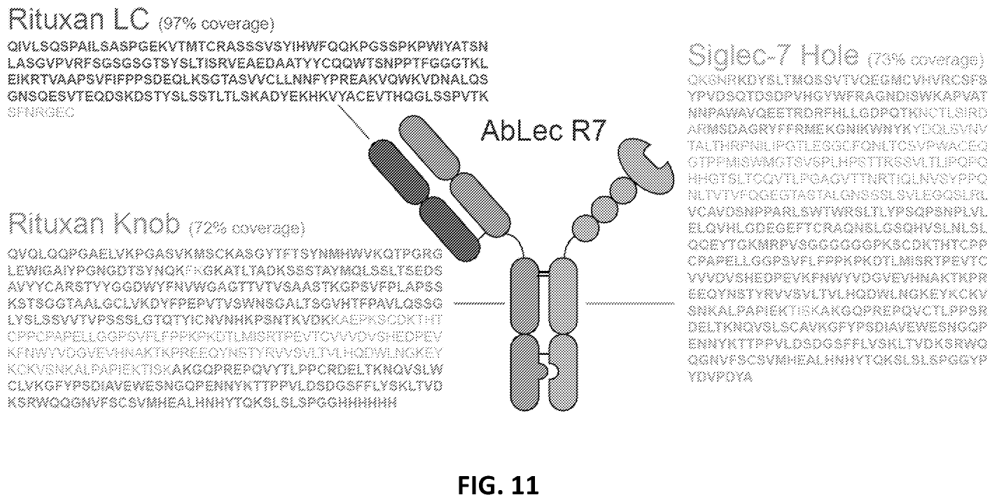

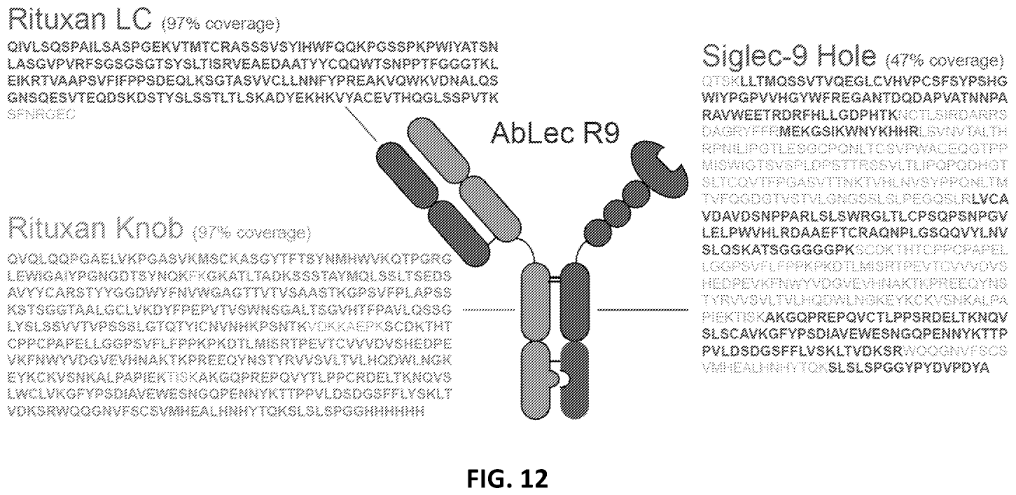

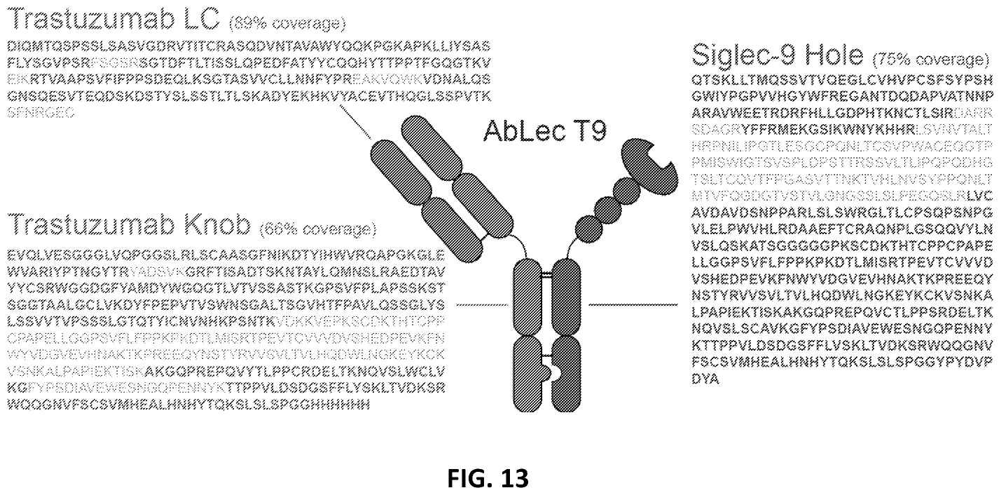

As initial proof of concept, a knobs-in-holes approach was used to generate a panel of recombinant Siglec-7 and -9-Fc chimeras that dimerize with monovalent antibody chains derived from FDA-approved antibodies (trastuzumab and rituximab) targeting the common cancer antigens HER2 and CD20, respectively. These constructs are schematically illustrated, and their amino acid sequences are provided, in FIGS. 11-14.

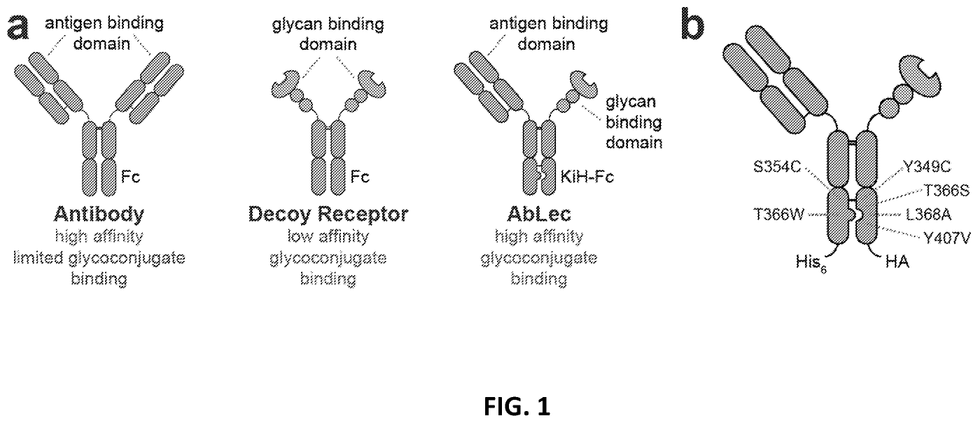

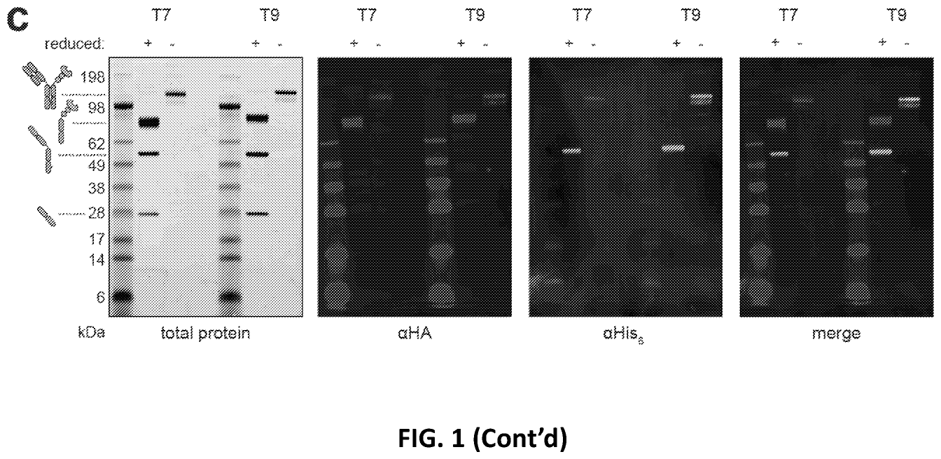

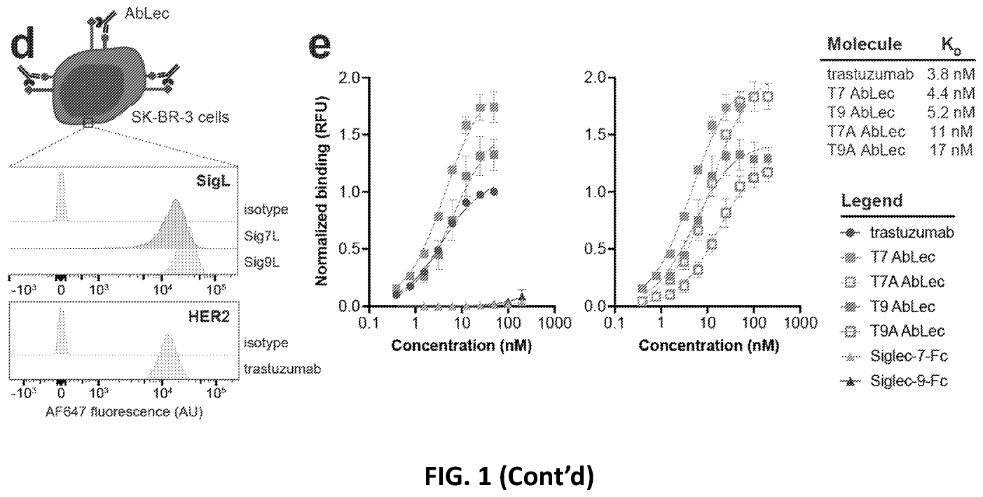

Provided in FIG. 1a-1e are schematic illustrations and data demonstrating that antibody-lectin (AbLec) bispecifics enable use of lectin decoy receptors for checkpoint blockade. (a) AbLecs combine the beneficial properties of monoclonal antibodies (high affinity and selectivity for desired tumor, immune cell, or tissue targets) with lectin decoy receptors (selectivity and specificity for cognate glycoconjugate ligands) while overcoming the limitations of each platform. (b) AbLecs were designed using a modified knobs-into-holes strategy using an IgG1 antibody framework. (c) Coexpression of trastuzumab heavy and light chains with Siglec-7-Fc or Siglec-9-Fc chains in Expi293 cells resulted in expression of a single protein product. Reducing SDS-PAGE analysis showed that these products were composed of 3 disulfide bonded protein chains consistent with the molecular weights of the Siglec-Fc chain, as well as the trastuzumab heavy and light chains. Western blotting against HA and His₆ tags on the Siglec-Fc and antibody heavy chains, respectively, further demonstrated that the full-length protein product was composed of both antibody and decoy receptor arms. Taken together, this evidence suggests that AbLecs are bifunctional heterodimers composed of both antibody and decoy receptor arms. (d) Dissociation constant (K_{D}) values for trastuzumab×Siglec-7 (T7) and trastuzumab×Siglec-9 AbLecs were measured by quantifying binding to SK-BR-3 cells at various concentrations via flow cytometry. SK-BR-3 cells express the HER2 antigen bound by trastuzumab as well as ligands for Siglecs-7 and -9 by flow cytometry. (e) The decoy receptor molecules (Siglec-7/9-Fc) bind only at low levels to SK-BR-3 cells, even at the highest concentrations tested (200 nM) and despite the fact that SK-BR-3 cells express Siglec-7 and -9 ligands (FIG. 1d). However, by combining the decoy receptor arm with the high affinity trastuzumab antibody arm, AbLecs can bind to SK-BR-3 cells at therapeutically relevant concentrations (left). AbLecs exhibit low nM K_{D} values similar to that of the parent antibody, trastuzumab (right). Further, AbLec binding is cooperative. By mutating a conserved arginine residue in the Siglec-Fc binding site to an alanine, created were T7A and T9A AbLec mutants with Siglec-Fc arms that exhibit significantly reduced affinities for Siglec ligands, as previously reported. The mutant AbLecs exhibited reduced binding to SK-BR-3 cells (middle), and resulted in a ˜2-3 fold increase in apparent K_{D} compared to WT AbLecs (right). This suggests that despite the low affinity of the Siglec-Fc decoy receptor molecule, when it accumulates at high local concentrations on the cell surface by virtue of the high affinity antibody-antigen interaction, it can bind to Siglec ligands. The observed contribution of the Siglec-Fc arm to binding may explain why AbLec dissociation constants are of the same order of magnitude as the divalent parent antibody trastuzumab, despite having only one antibody arm.

### Example 2—AbLecs Block Binding of Targeted Glycan-Binding Immunoreceptors

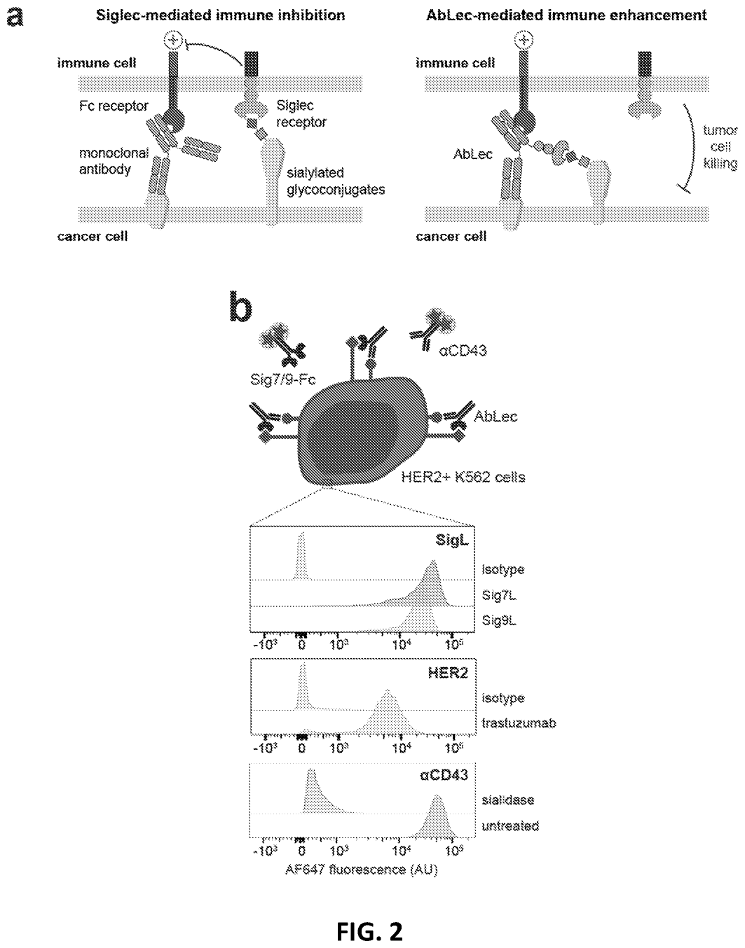

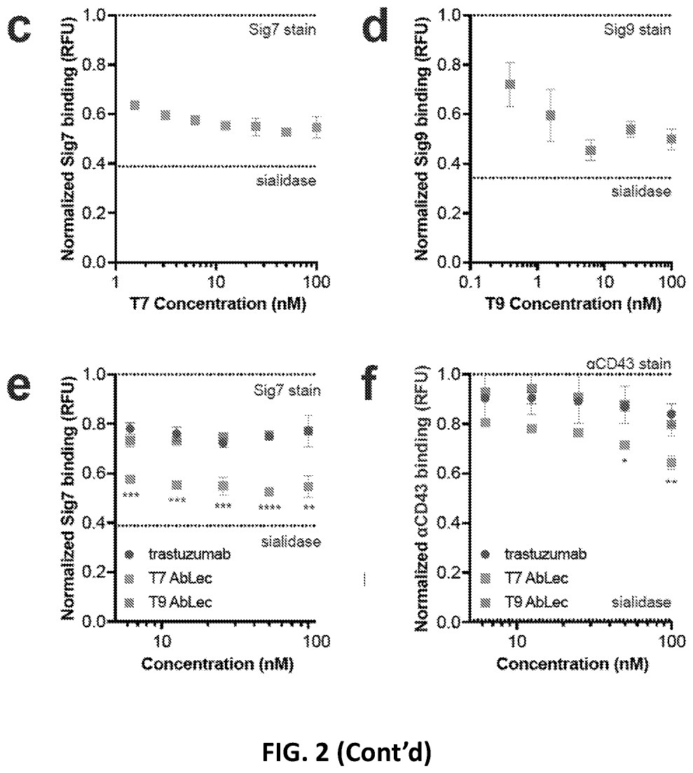

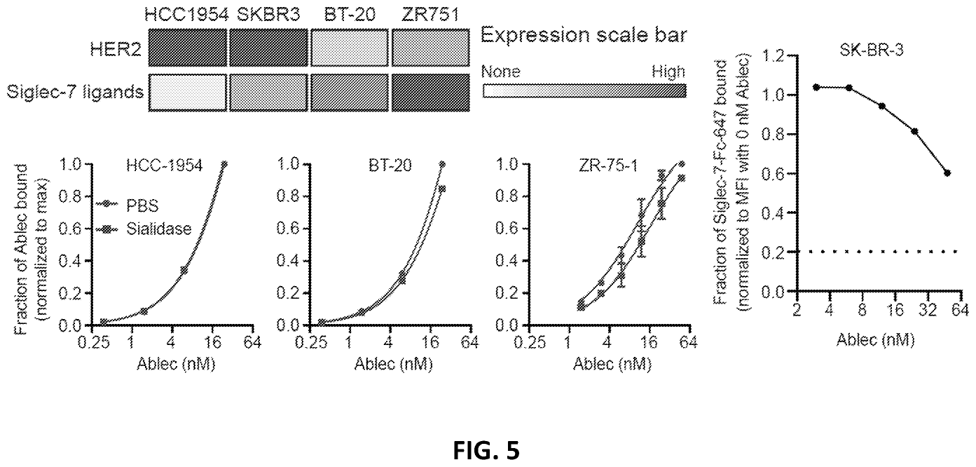

Demonstrated in this example is that AbLecs block binding of targeted glycan-binding immunoreceptors. Schematic illustrations and data are provided in FIG. 2a-2f and FIG. 5. (a) It was hypothesized that if AbLecs are able to relieve inhibitory signaling via the Siglec axis by blocking Siglec ligands on tumor cells in addition to recruiting immune cells via antibody effector functions, they have the potential to enhance anti-tumor immune responses compared to parent monoclonal antibodies. (b) Dissociation constant (K_{D}) values for trastuzumab/Siglec-7 (T7) and trastuzumab/Siglec-9 AbLecs were measured by quantifying binding to SK-BR-3 cells at various concentrations via flow cytometry. SK-BR-3 cells express the HER2 antigen bound by trastuzumab as well as ligands for Siglecs-7 and -9 by flow cytometry. Tested was the ability of T7 and T9 AbLecs to compete with AF647-labeled Siglec-Fc reagents for binding to HER2+K562 cells, which express the targeted HER2 antigen as well as ligands for Siglecs-7 and -9. It was observed that treatment of cells with increasing concentrations of T7 or T9 AbLec enhanced our ability to block binding of Siglec-7-Fc-AF647 (c) or Siglec-9-Fc-AF647 (d), respectively by flow cytometry. AbLecs reduced Siglec-Fc binding nearly to the level of the sialidase control, suggesting that they are able to block the vast majority of sialic acid-dependent binding of Siglec receptors to cells. (e) AbLecs further appear to only block binding of the targeted Siglec. Treatment with the T7 AbLec was able to block binding of the cognate Siglec-7-Fc to a greater extent compared to treatment with trastuzumab or the non-cognate T9 AbLec at all concentrations tested. (f) AbLecs bind and block the same epitopes bound by endogenous receptors. Tested was the ability of AbLecs to compete with the anti-CD43 antibody MEM59 for binding to HER2+K562 cells. It has previously been shown that the predominant ligand for Siglec-7 expressed on K562 cells is the mucin glycoprotein CD43, and that MEM59 can block binding of Siglec-7 to a sialylated epitope on CD43. It was observed that the T7 AbLec was able to block binding of MEM59, suggesting that the T7 AbLec binds and blocks the same CD43 epitope bound by the endogenous Siglec-7 immunoreceptor. Further, the T7 AbLec was able to block binding of MEM59 to a greater extent than trastuzumab or the non-cognate T9 AbLec.

FIG. 5 shows that trastuzumab hybrid AbLecs bind to diverse HER2+ human tumor cell lines and block binding of Siglec receptors. The plots on the left hand side of the figure show that the T7 AbLec binds to HCC-1954, SK-BR-3, BT-20, and, ZR-75-1 cell lines that express varying levels of the targeted HER2 antigen and ligands for Siglec-7. The graph on the right hand side of the figure shows that as we treat SK-BR-3 cells with increasing concentrations of T7 AbLec enhanced our ability to block binding of Siglec-7-Fc-AF647 to cells. The T7 AbLec reduced Siglec-Fc binding nearly to the level of the sialidase control, suggesting that AbLecs are able to block the vast majority of sialic acid-dependent binding of Siglec receptors to cells.

### Example 3—AbLecs Enhance Antibody-Dependent Cellular Phagocytosis and Cytotoxicity In Vitro

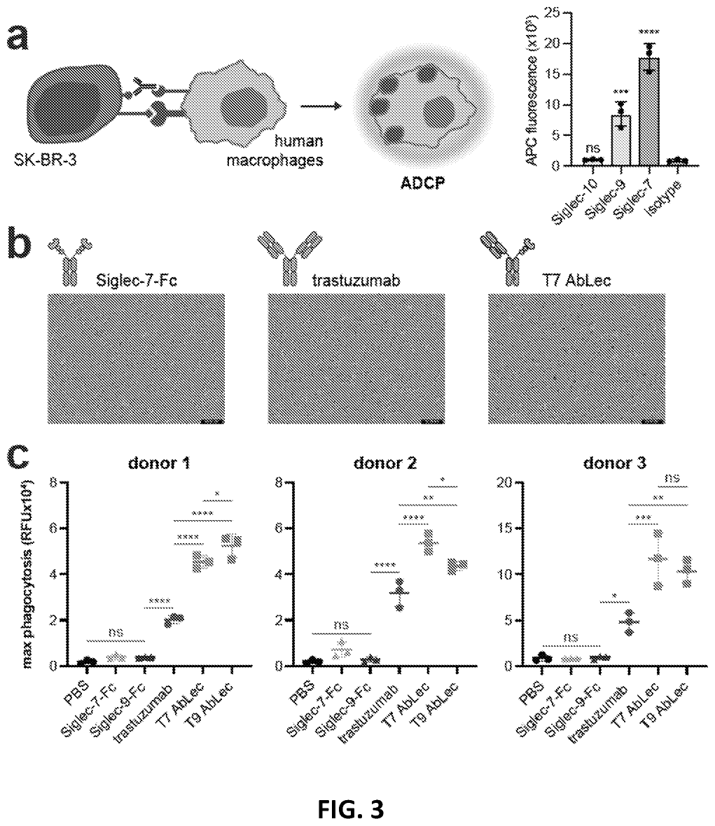

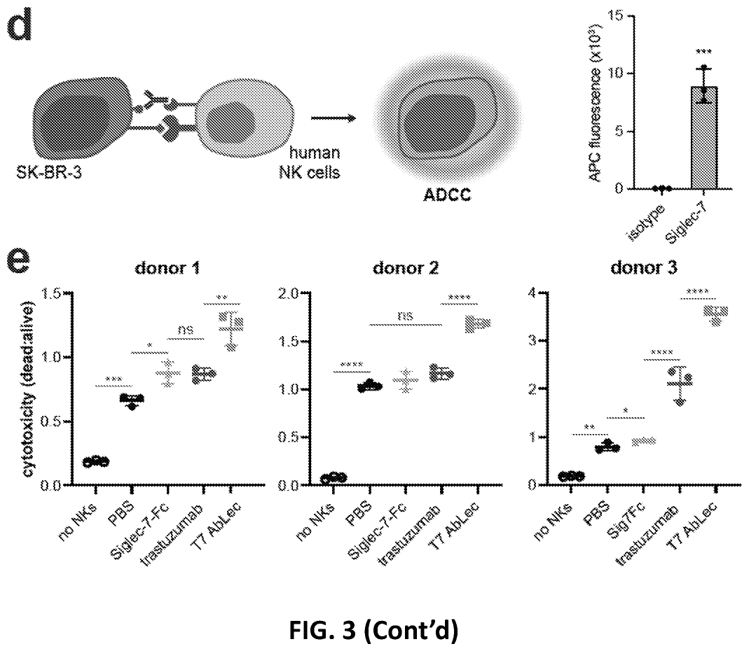

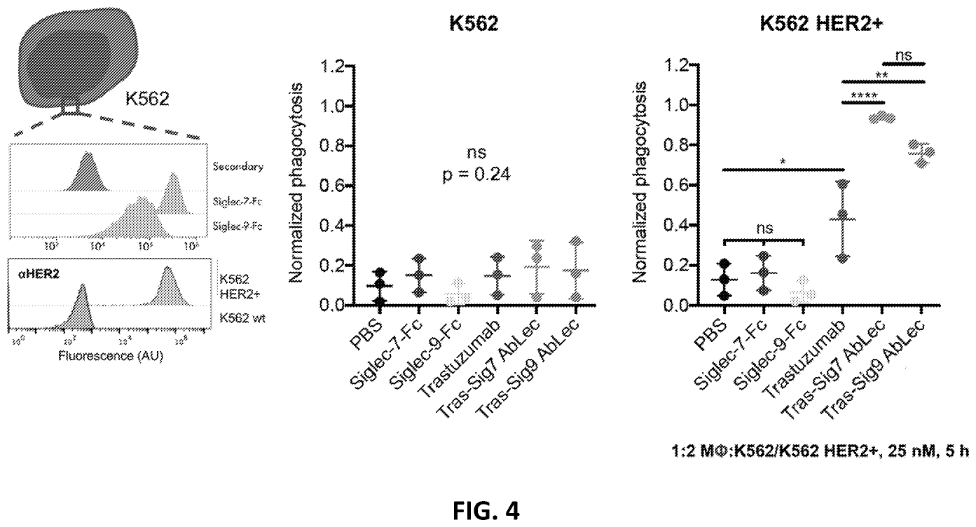

Demonstrated in this example is that AbLecs enhance antibody-dependent cellular phagocytosis and cytotoxicity in vitro. Schematic illustrations and data are provided in FIGS. 3a-3e and FIG. 4. (a) In vitro antibody-dependent cellular phagocytosis (ADCP) assays were performed using human macrophages isolated and differentiated from healthy donor peripheral blood. At the time of the assay, macrophages expressed Siglecs-7 and -9. SK-BR-3 target cells were labeled with pHrodo red to enable quantification of ADCP via time lapse fluorescence microscopy using an Incucyte instrument. (b) Images of macrophage/SK-BR-3 co-culture experiments after 5 h of incubation show levels of red fluorescence as an indicator of phagocytosis. (c) Across n=3 unique donors, T7 and T9 AbLecs significantly enhance ADCP of SK-BR-3 cells compared to trastuzumab or Siglec-Fcs alone. (d) In vitro antibody-dependent cellular cytotoxicity (ADCC) assays were performed using human NK cells isolated from healthy donor peripheral blood. At the time of the assay, NK cells expressed Siglec-7. SK-BR-3 target cells were labeled with celltracker red and co-cultured with NK cells in the presence of Sytox green to enable assessment of ADCC activity via quantification of target cell death by flow cytometry. (e) Across n=3 unique donors, the T7 AbLec significantly enhanced ADCC of SK-BR-3 cells compared to trastuzumab or Siglec-7-Fc alone.

FIG. 4 shows that AbLec-mediated enhancement of ADCP is dependent on expression of the targeted antigen (e.g., HER2). Across n=3 unique donors, the T7 and T9 AbLecs significantly enhanced ADCP of HER2+K562 cells compared to trastuzumab or Siglec-7/9-Fc alone. However, for WT K562 cells that do not express HER2, we observed no ADCP activity with either trastuzumab or AbLecs. This suggests that AbLec immunotherapy can be directed specifically to cells expressing antigens of interest (e.g., tumor antigens, immune cell markers, etc.).

### Example 4—AbLecs Outperform Combination Immunotherapy Via Siglec-Dependent Enhancement of Anti-Tumor Immune Responses In Vitro

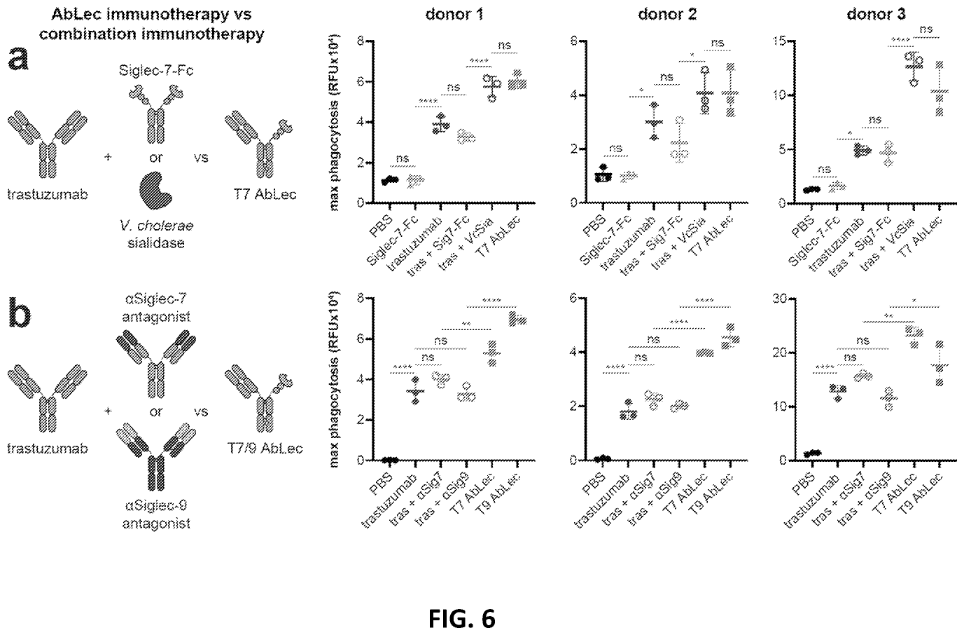

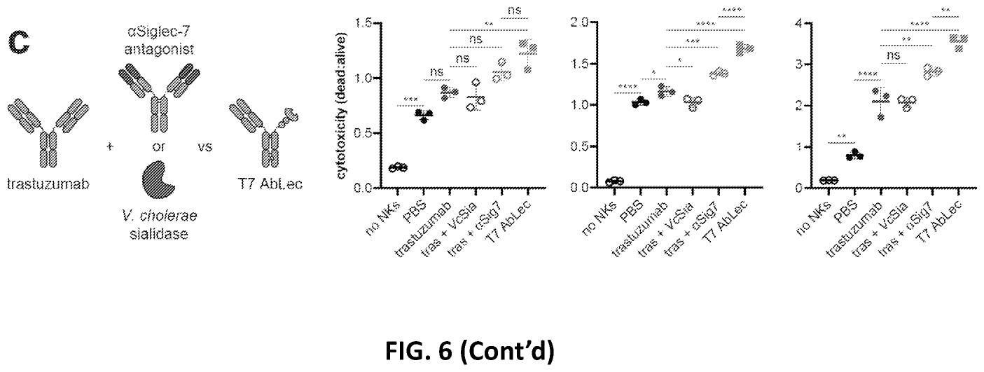

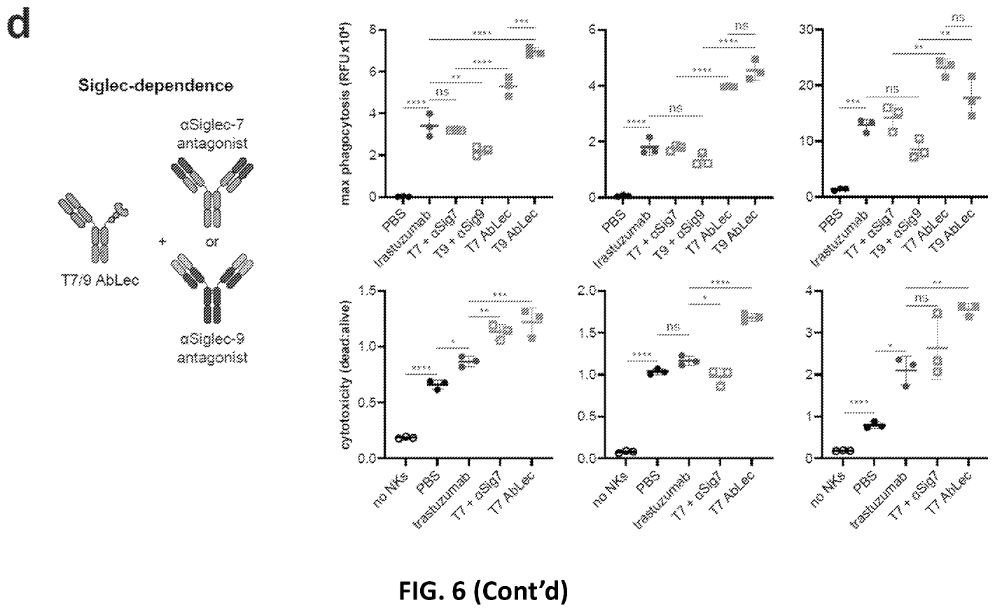

Demonstrated in this example is that AbLecs outperform combination immunotherapy via Siglec-dependent enhancement of anti-tumor immune responses in vitro. Schematic illustrations and data are provided in FIGS. 6a-6d. (a) T7 and T9 AbLecs elicited enhanced ADCP of HER2+K562 cells compared to the combination of trastuzumab with Siglec-7-Fc, Siglec-9-Fc, or V. cholerae sialidase across n=3 unique donors. T7 and T9 AbLecs elicited enhanced ADCP (b) and ADCC (c) of SK-BR-3 cells compared to the combination of trastuzumab with Siglec-7 or -9 antagonist antibodies, or V. cholerae sialidase across n=3 unique donors. (d) AbLec-mediated enhancement of ADCP and ADCC was Siglec-dependent. If macrophages (top) or NK cells (bottom) were incubated with Siglec-7 or -9 antagonist antibodies prior to co-culture with SK-BR-3 cells, essentially functioning as a Siglec-7/9 knockout in the immune cells, ADCP or ADCC levels observed upon AbLec treatment were reduced to similar levels as those observed with trastuzumab treatment. However, if Siglec immunoreceptors were not blocked, AbLecs enhanced in vitro ADCP and ADCC. This Siglec-dependence was observed across n=3 unique donors and suggests that the enhancement of ADCP and ADCC observed with AbLec treatment is a result of blockade of the targeted Siglec axis.

### Example 5—the AbLec Platform Enables Blockade of Diverse Glyco-Immune Checkpoint Targets

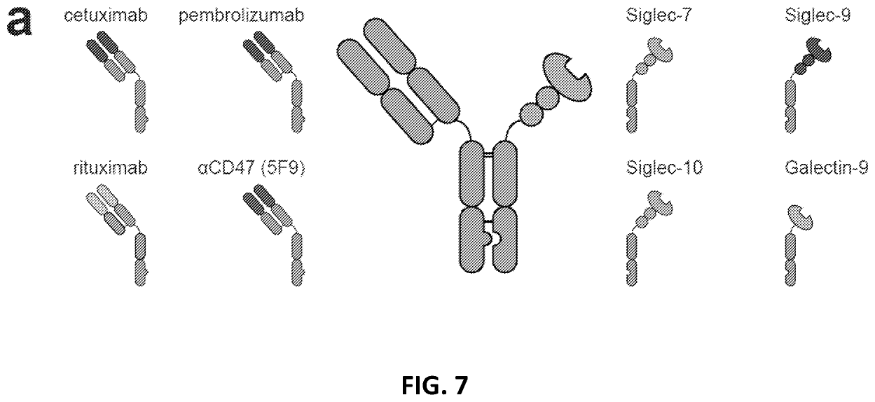

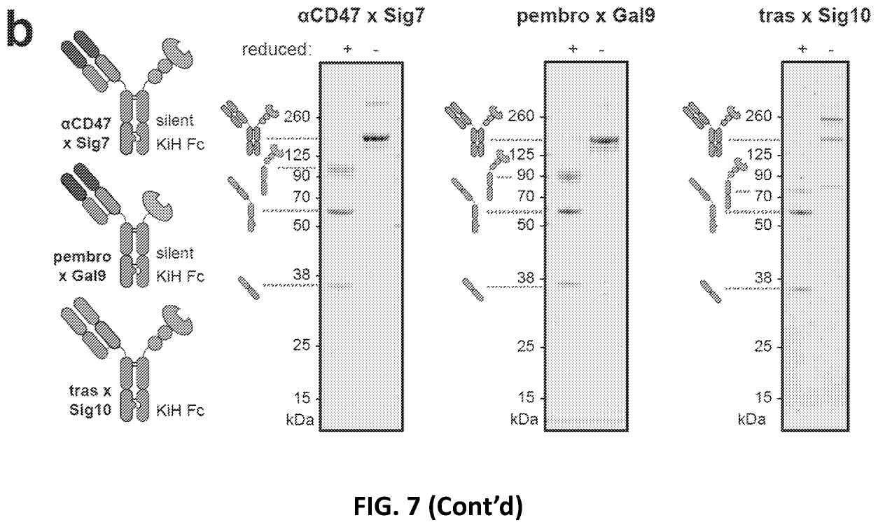

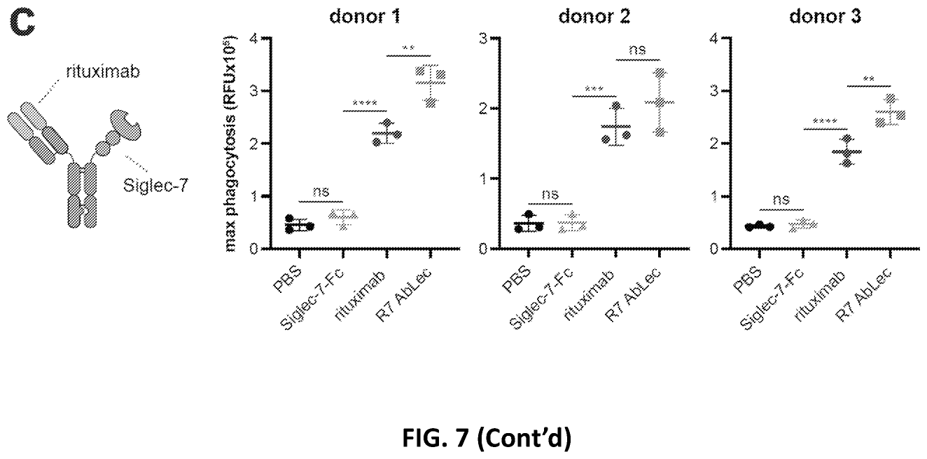

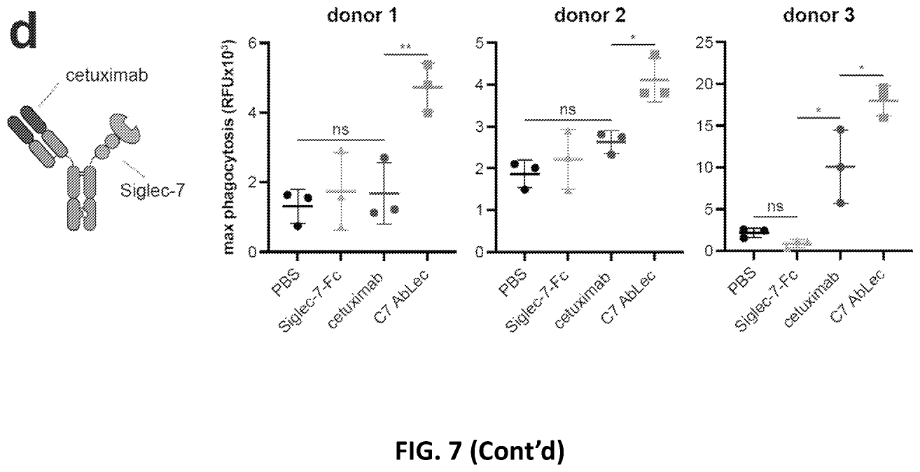

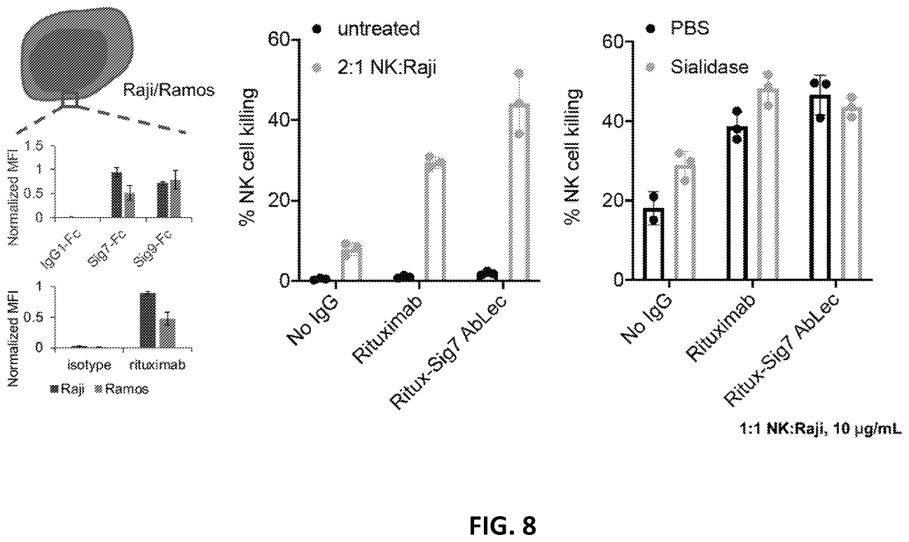

Demonstrated in this example is that the AbLec platform enables blockade of diverse glyco-immune checkpoint targets. Schematic illustrations and data are provided in FIGS. 7a-7d and FIG. 8. (a) Due to the modular architecture of knobs-into-holes bispecific scaffold used to make AbLecs, antibody and decoy receptor components can be readily exchanged for production of additional checkpoint inhibitors. (b) SDS-PAGE characterization of additional AbLec candidates with diverse mechanisms of action. The magrolimab×Siglec-7 AbLec is designed for dual checkpoint blockade of CD47 and Siglec-7 ligands on tumor cells. Magrolimab is an anti-CD47 antibody (Liu et al. 2015) currently in phase III clinical trials for hematological cancers (Garcia-Manero et al. 2021). The pembrolizumab×Galectin-9 AbLec is designed for dual checkpoint blockade of PD-1 and Galectin-9 ligands on exhausted T cells. Galectin-9 has been shown to contribute to immune evasion by binding TIM-3 checkpoint on T cells and contributing to T cell exhaustion (Yang et al. 2021). The trastuzumab×Siglec-10 AbLec is designed to simultaneously target HER2+ tumors and block the Siglec-10 immune checkpoint, which was recently shown to play roles in immune evasion in breast and ovarian cancers (Barkal et al. 2019). Functional characterization of rituximab×Siglec-7 (R7) and cetuximab×Siglec-7 (C7) AbLecs demonstrates the utility of the AbLec platform for combination tumor targeting and glyco-immune checkpoint blockade in diverse tumor types. Across n=3 unique donors, R7 (c) and C7 (d) AbLecs significantly enhance ADCP of CD20+ Ramos cells or EGFR+K562 cells compared to the parent antibody or Siglec-7-Fc alone.

FIG. 8 shows that, across n=3 unique donors, the R7 AbLec also enhances ADCC of CD20+ Raji cells compared to rituximab (left). Enhancement of ADCC was a sialic acid-dependent effect, as there was no significant enhancement of ADCC with the R7 AbLec compared to rituximab following treatment with sialidase (right).

### Example 6—Expression of Diverse AbLec Molecules

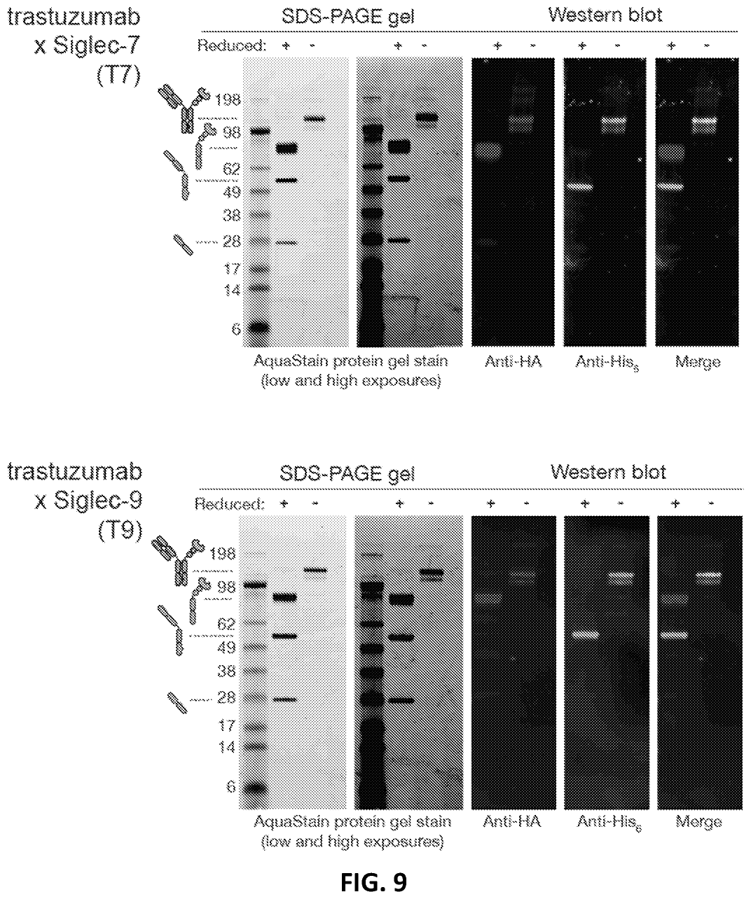

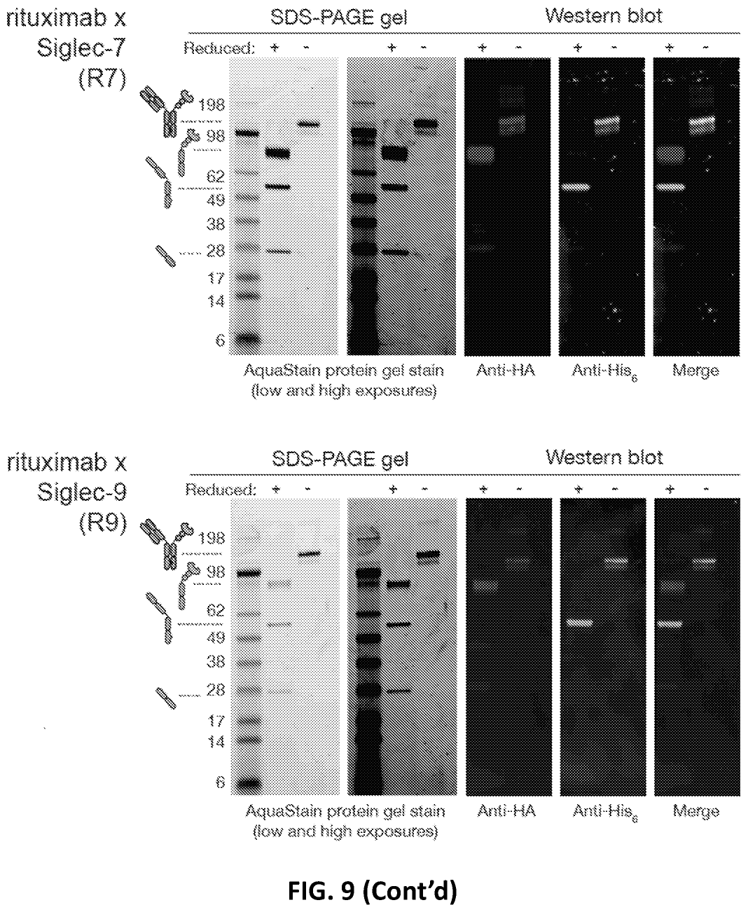

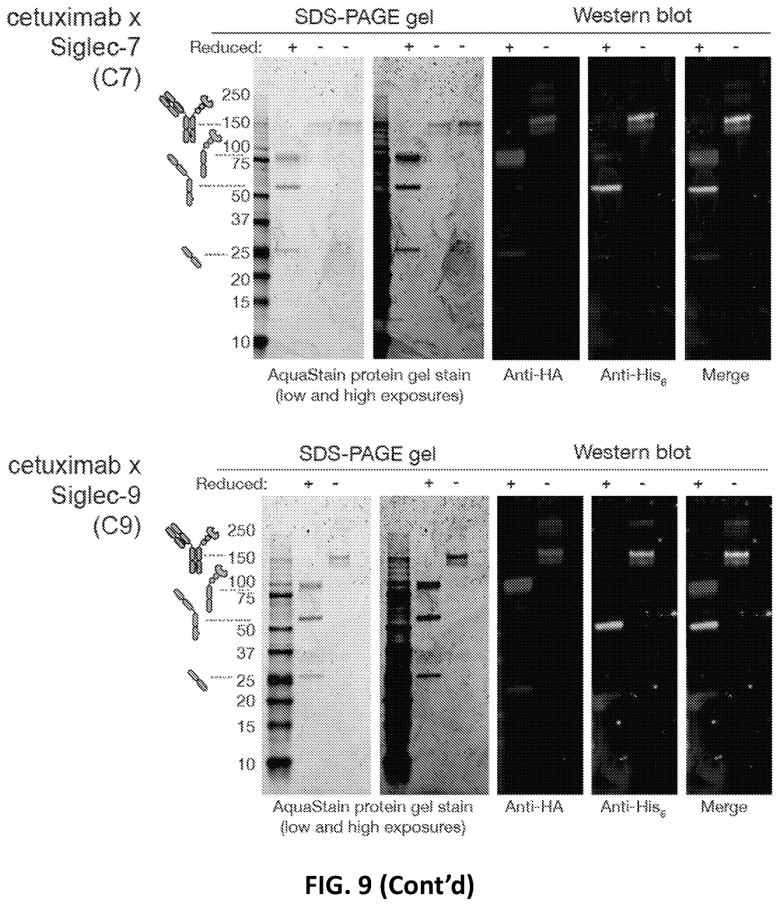

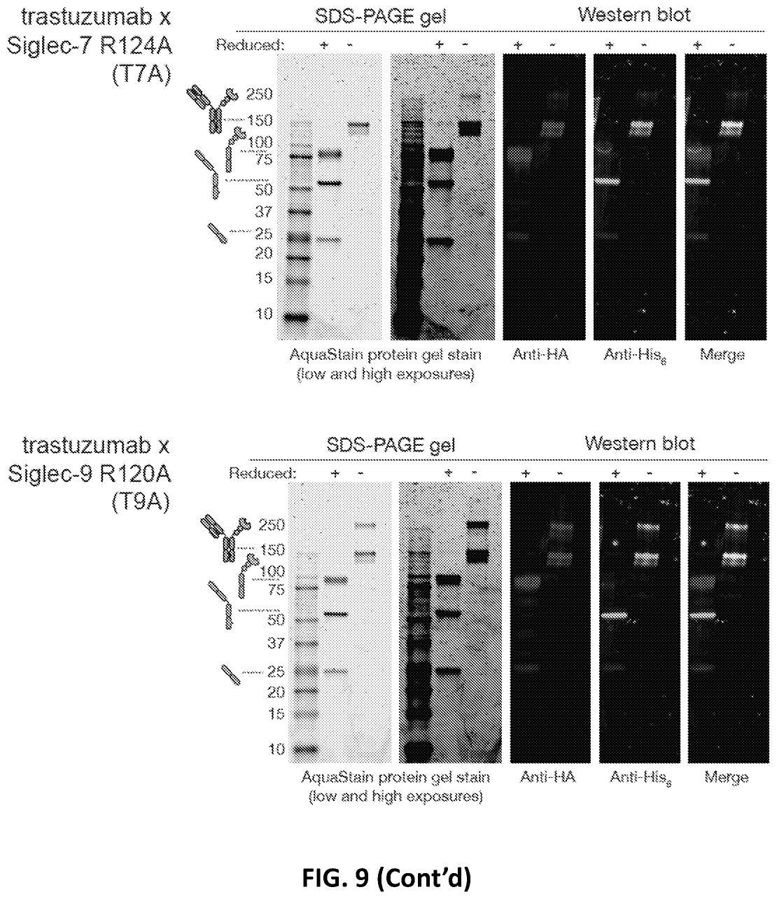

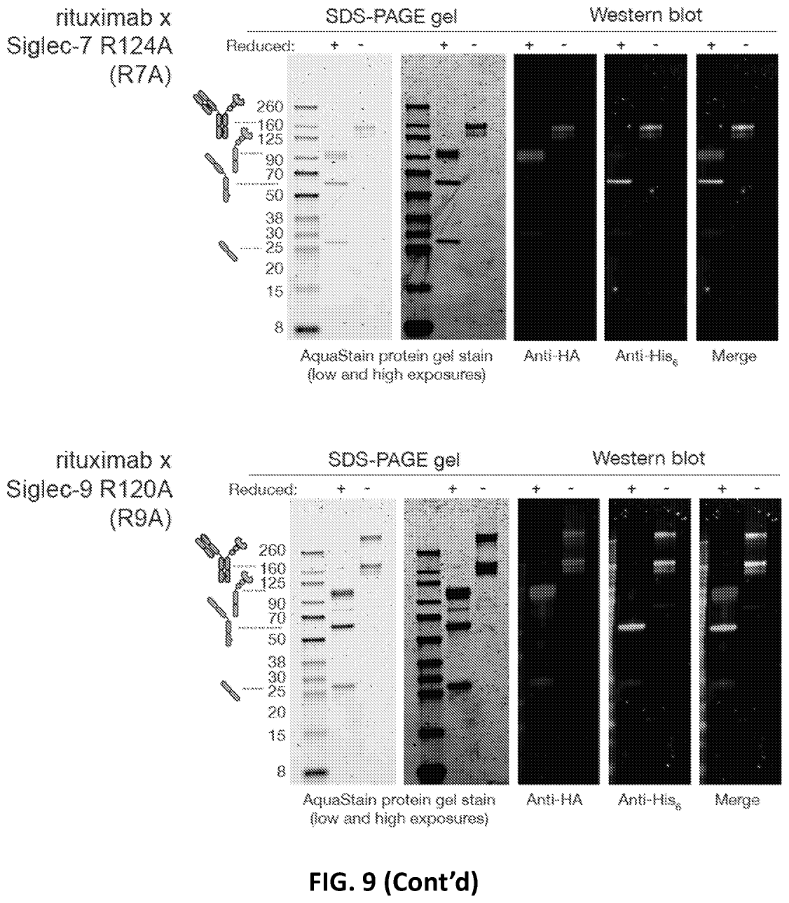

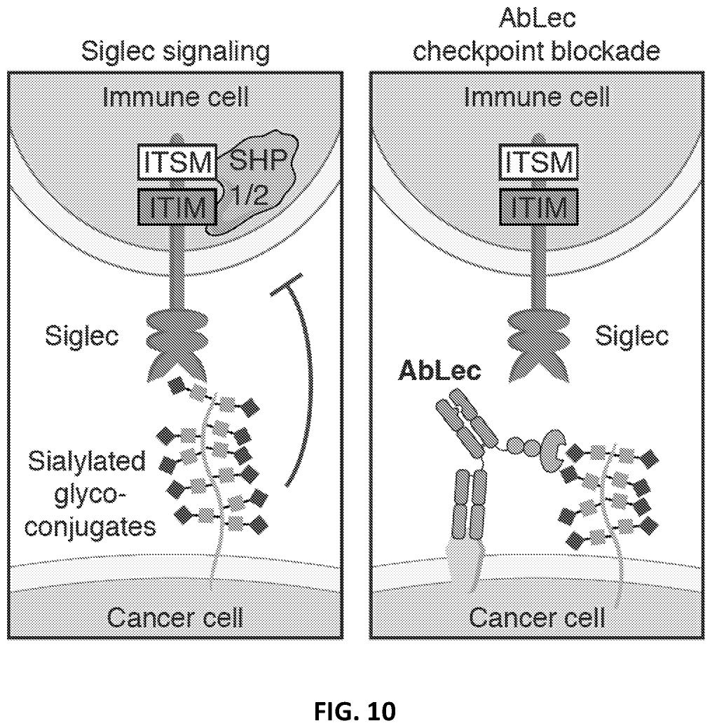

Shown in FIG. 9 are reducing and non-reducing SDS-PAGE and Western blot analyses for diverse AbLec molecules. Reducing SDS-PAGE demonstrates showed that each AbLec is composed of 3 disulfide bonded protein chains consistent with the molecular weights of the decoy receptor chain, as well as the antibody heavy and light chains. Western blotting against HA and His6 tags on the decoy receptor and antibody heavy chains, respectively, further demonstrated that full-length AbLecs are composed of both antibody and decoy receptor arms.

### Materials and Methods

### Plasmids and Protein Sequences

All AbLec plasmids were generated by Twist Bioscience. DNA Sequences are listed in Table 1 and protein sequences in Table 2. AbLec sequences were inserted into the Twist Bioscience vector pTwist CMV BetaGlobin, using the XhoI and NheI cut sites. The trastuzumab used in this paper was expressed from a pCDNA3.1 vector described in our previous work (Gray et al. 2020). The rituximab and cetuximab antibody variable sequences were generated by IDT and cloned into the variable regions of the VRC01 antibody plasmid vector (a generous gift from the Kim lab at Stanford) by using the In-Fusion cloning kit (Takara) according to the manufacturer's protocol.

### Protein Expression and Purification

Antibodies and AbLecs were expressed in the Expi293F system (Thermo Fisher) and expressed according to established manufacturer protocol. For the rituximab and cetuximab antibodies, a 1:1 heavy to light chain plasmid ratio by weight was used. For the AbLecs, a 2:1:1 ratio of lectin:heavy chain:light chain was used. The trastuzumab antibody heavy chain and light chain were co-expressed from a single plasmid. After seven days of expression, proteins were collected from the supernatant by pelleting cells at 300×g for 5 min, followed by clarification with a spin at 3700×g for 40 min, and filtration through a 0.2 μm nylon filter (Fisher Scientific 0974025A). Antibodies were purified by manual gravity column using protein A agarose (Fisher Scientific 20333) by flowing the clarified supernatant through the column 2×, sialidase treating on the beads with 2 μM ST sialidase for 0.5-2 h rt, then washing with 5× column volumes of PBS, and eluting 5 mL at a time with 100 mM glycine buffer pH 2.8 into tubes pre-equilibrated with 150 uL of 1 M Tris pH 8. Antibodies were buffer exchanged into PBS using PD-10 columns (GE). AbLecs were purified by manual gravity column using nickel-NTA agarose resin (Qiagen 30210). Briefly, AbLec supernatant was incubated with the resin (pre-equilibrated in PBS) for ˜ 1 hour at 4 C. Beads and supernatant were then loaded onto a chromatography column (BioRad 7321010), sialidase treated on the beads with 2 μM ST sialidase for 2 h rt, washed with 20× column volumes PBS+20 mM imidazole, and eluted twice with 5× column volumes PBS+250 mM imidazole. AbLecs were buffer exchanged into PBS using PD-10 desalting columns.

### Bioconjugation to Make Siglec-Fc-AF647 Reagents

Siglec-Fc DNA sequences were expressed in Expi293F cells that co-express stable human FGE protein for aldehyde tagging. Cells were expressed in the dark according to the Expi293 protocol from Thermo Fisher, then filtered through a 0.2 μm filter and loaded onto a column containing protein A agarose beads. Protein was sialidase-treated on the column for 2 h (2 μM Salmonella typhimurium sialidase, rt). Followed by washing with PBS and elution with 100 mM glycine (pH 2.8), 10 mL, into buffered Tris pH 8 solution. Siglecs were buffer exchanged into acidic buffer, concentrated, and conjugated with HIPS-azide according to the protocol from Gray, et al (Gray et al. 2020). Siglec-Fc-azide was then taken without further characterization, buffer exchanged into PBS, and 100× molar equivalents of DBCO-AF647 (Click Chemistry Tools, 1302-1) in DMSO were added and the reaction was mixed at 500 rpm in the dark for 2 hours rt. Siglecs were buffer exchanged by 6× centrifugation on Amicon columns (30 kDa MWCO) in PBS, and AF647 addition was confirmed by using a NanoDrop spectrophotometer at 650 nm for the AF647 dye (extinction coefficient 239,000) and at 280 nm for the protein (using extinction coefficients calculated for each Siglec by Expasy).¹⁸

### AbLec Gel Characterization

For SDS-PAGE gels, 2 μg of protein with SDS dye alone (non-reducing conditions), or SDS dye+1 M betamercaptoethanol, heated at 95 C for 5 min (reducing conditions), were loaded onto a Bis-Tris 4-12% Criterion™ XT Bis-Tris Protein Gel, 18 well, (Bio-Rad 3450124) and run with XT-MES buffer at 180 V for 40 min. Proteins were stained with Aquastain (Bulldog Bio AS001000) for 10 minutes followed by a 10 minute destain in water. For western blotting of the AbLecs, 0.2 μg of protein was loaded onto an SDS-PAGE gel and run as described above, then the gel was transferred to a nitrocellulose membrane using the Trans-Blot® Turbo™ RTA Midi Nitrocellulose Transfer Kit (Bio-Rad 1704271), 25V, 14 min. The membrane was blocked in blocking buffer (PBS+0.5% BSA) for 1 hour rt, then stained with Invitrogen HA Tag Polyclonal Antibody (SG77) (Thermo Fisher Scientific 71-5500) and Purified anti-His Tag antibody (BioLegend 652502) for 1 hour in blocking buffer shaking at rt. The membrane was washed 3× in PBST (PBS+0.1% Tween), followed by staining with secondary antibodies IRDye® 800CW Goat anti-Mouse and IRDye® 680RD Goat Anti-Rabbit (LI-COR) in PBST for 15 minutes shaking at room temp, followed by 3× more washes in PBST. All gels were imaged on an Odyssey® CLx Imaging System (LI-COR).

### AbLec Melting Temperature Characterization

SYPRO orange dye (Thermo Fisher), was diluted to make a 25× stock, and 5 uL (final 5×) concentration with the antibodies) was added to 20 uL of AbLec, Siglec-Fc, or antibody at 0.2 mg/mL in PBS to make 25 uL per well in a 96 well qPCR plate. Denaturation of proteins was analyzed in the FRET fluorescence channel in the qPCR by increasing the temperature 0.5° C. every 1 min from 25° C. to 95° C.

### AbLec Mass Spectrometry Characterization

Four micrograms of each AbLec dissolved in PBS were digested with trypsin for proteomic analysis. Samples were incubated for 18 minutes at 55° C. with 5 mM dithiothreitol, followed by a 30-minute incubation at room temperature in the dark with 15 mM iodoacetamide. Trypsin (Promega) was added at a 1:20 w:w ratio and digestions proceeded at room temperature overnight. The following morning, samples were desalted by first quenching the digestion with formic acid to a final pH of ˜2, followed by desalting over a polystyrene-divinylbenzene solid phase extraction (PS-DVB SPE) cartridge (Phenomenex, Torrance, CA). Samples were dried with vacuum centrifugation following desalting and were resuspended in 0.2% formic acid in water at 0.5 μg per μL.

Approximately 1 μg of peptide was injected per analysis, wherein peptides were separated over a 25 cm EasySpray reversed phase LC column (75 μm inner diameter packed with 2 μm, 100 Å, PepMap C18 particles, Thermo Fisher Scientific). The mobile phases (A: water with 0.2% formic acid and B: acetonitrile with 0.2% formic acid) were driven and controlled by a Dionex Ultimate 3000 RPLC nano system (Thermo Fisher Scientific). Gradient elution was performed at 300 nL/min. Mobile phase B was held at 0% over 6 min, followed by an increase to 5% at 7 minutes, 25% at 66 min, a ramp to 90% B at 70 min, and a wash at 90% B for 5 min. Flow was then ramped back to 0% B at 75.1 minutes, and the column was re-equilibrated at 0% B for 15 min, for a total analysis time of 90 minutes. Eluted peptides were analyzed on an Orbitrap Fusion Tribrid MS system (Thermo Fisher Scientific). Precursors were ionized using an EASY-Spray ionization source (Thermo Fisher Scientific) source held at +2.2 kV compared to ground, and the column was held at 40° C. The inlet capillary temperature was held at 275° C. Survey scans of peptide precursors were collected in the Orbitrap from 350-1350 m/z with an AGC target of 1,000,000, a maximum injection time of 50 ms, and a resolution of 60,000 at 200 m/z. Monoisotopic precursor selection was enabled for peptide isotopic distributions, precursors of z=2-5 were selected for data-dependent MS/MS scans for 2 seconds of cycle time, and dynamic exclusion was set to 30 seconds with a ±10 ppm window set around the precursor monoisotope. An isolation window of 1 m/z was used to select precursor ions with the quadrupole. MS/MS scans were collected using HCD at 30 normalized collision energy (nce) with an AGC target of 100,000 and a maximum injection time of 54 ms. Mass analysis was performed in the Orbitrap a resolution of 30,000 at 200 m/z and scan range set to auto calculation.

Raw data were processed using Byonic¹⁹, version MaxQuant version 3.11.3. Oxidation of methionine (+15.994915) was set as a common²⁰ variable modification, protein N-terminal acetylation (+42.010565) and asparagine deamination (+0.984016) were specified as rare1 variable modifications, and carbamidomethylation of cysteine (+57.021464) was set as a fixed modification. Up to three common and two rare modifications were permitted. A precursor ion search tolerance of 10 ppm and a product ion mass tolerance of 20 ppm were used for searches, and three missed cleavages were allowed for full trypsin specificity. Peptide spectral matches (PSMs) were made against custom FASTA sequence files that contained appropriate combinations of Siglec-7 and -9 holes, and Trastuzumab/rituximab knobs and light chains. Peptides were filtered to a 1% false discovery rate (FDR) and a 1% protein FDR was applied according to the target-decoy method.2 All peptide identifications were manually inspected, and sequences coverages were calculated only from validated peptide identifications. Sequence coverage percentages are derived from the proportion of amino acids explained by peptide identifications relative to the total number of amino acids.

### Cell Culture

All cell lines were purchased from the American Type Culture Collection (ATCC). SK-BR-3, HCC-1954, K562, Raji, and Ramos were cultured in RPMI+10% heat-inactivated fetal bovine serum (FBS) without antibiotic selection. Expi293F cells were a gift from the Kim lab at Stanford and were cultured according to Thermo Fisher Scientific's user guide. K562s were transfected according to manufacturer's protocol with EGFR using pre-packaged lentiviral particles (G&P Biosciences) and selected for EGFR expression by culture in 1 μg/mL puromycin (InVivoGen). K562s were transfected according to manufacturer's protocol with CD20 using pre-packaged lentiviral particles (G&P Biosciences LTV-CD20) and sorted for CD20-expression using rituximab and a BV421-labeled anti-human secondary (Biolegend) as the staining reagent on a FACS instrument. HER2 WT was a gift from Mien-Chie Hung (Addgene plasmid #16257) and stable HER2+ cell lines were generated following their protocol, 21 protein expression was verified by flow cytometry. Cell lines were not independently authenticated beyond the identity provided from the ATCC. Cell lines were cultivated in a humidified incubator at 5% CO₂ and 37° C. and tested negative for mycoplasma quarterly using a PCR-based assay.

### Quantifying AbLec K_{D}

HER2+K562 and SK-BR-3 cells were isolated from the cell culture supernatant or via dissociation with TrypLE (Gibco), respectively, washed with 1×PBS, and resuspended in blocking buffer. 60,000 cells were then distributed into wells of a 96-well V-bottom plate (Corning). Various concentrations (200-0.4 nM) of trastuzumab, Siglec-Fcs, or AbLecs were added to the cells in equal volumes and incubated with cells for 1 h at 4° C. with periodic pipet mixing. Cells were washed three times in blocking buffer, pelleting by centrifugation at 300 g for 5 min at 4° C. between washes. Cells were resuspended in AF647 Goat Anti-Human (BioLegend) in blocking buffer for 30 min at 4° C. Cells were further washed twice and resuspended in blocking buffer, and fluorescence was analyzed by flow cytometry (BD LSR II). Gating was performed using FlowJo v. 10.0 software (Tree Star) to eliminate debris and isolate single cells. Mean fluorescence intensity (MFI) of the cell populations were normalized to the MFI of cells stained with 50 nM trastuzumab from each experimental replicate. MFI values were fit to a one-site total binding curve using GraphPad Prism 9, which calculated the K_{D} values as the antibody concentration needed to achieve a half-maximum binding.

Competitive Binding with Siglec-Fc-AF647 Reagents

HER2+K562 and SK-BR-3 cells were isolated from the cell culture supernatant or via dissociation with TrypLE (Gibco), respectively, washed with 1×PBS, and resuspended in blocking buffer. Cells were aliquoted for sialidase treatment with 100 nM V. cholerae sialidase at 37° C. for 30 min in blocking buffer. 60,000 untreated or sialidase treated cells were then distributed into wells of a 96-well V-bottom plate (Corning). Various concentrations (100-0.4 nM) of trastuzumab or AbLecs with 200 nM Siglec-Fc-AF647 reagents were added to the cells in equal volumes and incubated with cells for 2-3 h at 4° C. with periodic pipet mixing. Cells were washed three times and resuspended in blocking buffer, pelleting by centrifugation at 300 g for 5 min at 4° C. between washes, and fluorescence was analyzed by flow cytometry (BD LSR II). Gating was performed using FlowJo v.10.0 software (Tree Star) to eliminate debris and isolate single cells. Mean fluorescence intensity (MFI) of the cell populations were normalized to the MFI of cells stained with 200 nM Siglec-Fc-AF647 without antibody or AbLecs from each experimental replicate. MFI values were fit to a one-site total binding curve using GraphPad Prism 9, which calculated the K_{D} values as the antibody concentration needed to achieve a half-maximum binding.

### Isolation of Donor NK Cells

PBMCs were isolated from LRS chambers as described above, and stocks were prepared at 2-4×10⁷ cells in 90% heat-inactivated FBS+10% DMSO and stored in liquid nitrogen vapor until use. The day prior to use stocks were thawed, NK cells were isolated using the EasySep NK isolation kit (StemCell Technologies 17955), and cells were cultured overnight with 0.5 μg/mL recombinant IL-2 (Biolegend 589106) in RPMI+10% heat-inactivated FBS until use.

### NK Cell Flow Cytometry

Following 24 h activation with IL-2, NK cells were collected from culture supernatant, washed with 1×PBS and resuspended in blocking buffer. On the day of analysis, macrophages were stained with anti-CD16, Siglec-7, and isotype controls in blocking buffer for 30 min at 4° C. After 2× washes in PBS, NK cells were analyzed by flow cytometry (BD LSR II) and gated for CD16 positive cells using FlowJo v10. NK cells were >85% pure by flow cytometry.

### NK Cell Killing Assays

Target cells were lifted stained with celltracker deep red dye according to manufacturer's protocol. NK cells and target cells were mixed at an effector to target (E/T) ratio of 4:1 and Sytox Green (Thermo) was added at 100 nM. Cell death was analyzed by flow cytometry by selecting the red (FL4-A⁺) cells and calculating the percent dead as Sytox Green⁺/total red cells. Replicates from three unique blood donors were plotted in Prism 9.0 (GraphPad Software, Inc).

### Isolation and Differentiation of Donor Macrophages

LRS chambers were obtained from healthy anonymous blood bank donors and PBMCs were isolated using Ficoll-Paque (GE Healthcare Life Sciences) density gradient separation. Monocytes were isolated by plating ˜1×10⁸ PBMCs in a T75 flask of serum-free RPMI for 1-2 hours, followed by 3× rigorous washes with PBS+Ca+Mg to remove non-adherent cells. The media was then replaced with IMDM with 10% Human AB Serum (Gemini), to differentiate the macrophages for 7-9 days prior to their use in a phagocytosis or flow cytometry experiment.

### Macrophage Flow Cytometry

Macrophages on day 7-9 were lifted from the plate as described above, then fixed for 15 min with 4% formaldehyde (Thermo) in PBS, and washed 3× in PBS and stored at 4° C. for 2-7 days until analysis. On the day of analysis, macrophages were stained with CD11b, CD14, Siglecs-7, -9, -10, and an isotype control in blocking buffer for 30 min at 4 ºC. After 2× washes in PBS, macrophages were analyzed by flow cytometry on an LSR II instrument and gated for CD11b and CD14 double positive cells using FlowJo v10. Macrophages were >85% pure by flow cytometry.

### Phagocytosis Assays

Macrophages were washed with PBS and lifted by 20 min incubation at 37° C. with 10 mL TrypLE (Thermo). RPMI+10% HI FBS was added to equal volume, and the macrophages were pelleted by centrifugation at 300×g for 5 min and resuspended in IncuCyte medium (phenol-red free RPMI+10% HI FBS). Macrophages (10,000 cells, 100 μL) were added to a 96 well flat-bottom plate (Corning) and incubated in a humidified incubator for 1 h at 37 ºC. Meanwhile, target cells were washed 1× with PBS, then treated with 1:80,000 diluted pHrodo red succinimydyl ester dye (Thermo Fisher) in PBS at 37 ºC for 30 min, washed 1× and resuspended in IncuCyte medium. Finally, 10 uL of 20× antibody or AbLec stocks in PBS were added to the macrophages, followed by the pHrodo red-stained target cells (90 uL, 20,000 cells). Cells were plated by gentle centrifugation (50×g, 2 min). Two images per well were acquired at 1 h intervals until the maximum signal was reached (5 hours for breast cancer cell lines and K562 cells, 2 hours for Raji and Ramos cells). The quantification of pHrodo red fluorescence was empirically optimized for phagocytosis of each cell line based on their background fluorescence and size. K562s were analyzed with a threshold of 0.8, an edge sensitivity of −70, and the area was gated to between 100 and 2000 μm² with integrated intensities between 300 and 2000 RCU×μm²/image. HCC-1954 were analyzed with a threshold of 1.5, an edge sensitivity of −45, and an area between 30 and 2000 μm². SK-BR-3 analysis had a threshold of 1, an edge sensitivity of −55, a minimum integrated intensity of 60, and a maximum area and eccentricity 3000 and 0.96, respectively. Ramos and Raji gating was defined using a threshold of 1.5, an edge sensitivity of −45, and areas between 100 and 2000 μm². The total red object integrated intensity (RCU×μm²/Image) was taken for each image. For each experiment, the maximum phagocytosis measured by pHrodo red was normalized to 1, and then triplicate technical well replicates were averaged for each biological replicate. Replicates from three unique blood donors were plotted in Prism 9.0 (GraphPad Software, Inc).

Acquisition of IncuCyte Images For phagocytosis and toxicity assays, images were obtained over time using an Incucyte® S3 Live-Cell Analysis System (Essen BioScience) within a Thermo Fisher Scientific tissue culture incubator at maintained 37° C. with 5% CO₂. Data were acquired from a 10× objective lens in phase contrast, a green fluorescence channel (ex: 460±20, em: 524±20, acquisition time: 300 ms), and from a red fluorescence channel (ex. 585±20, em: 665±40, acquisition time: 400 ms). Two images per well were acquired at intervals. Unless otherwise specified, all cells were analyzed by Top-Hat segmentation with 100 μm radius, edge split on, hole fill: 0 μm²

### Cell Growth and Toxicity Assays

GFP⁺ SK-BR-3, HER2+K562, and HCC-1954 cells were lifted with 2 mL trypsin for 5 min at 37° C., rinsed with 8 mL normal growth media and cells were pelleted by centrifugation at 300×g and resuspended in phenol-red-free growth medium containing 50 nM Sytox green cell dead stain (Thermo Fisher) or 5 nM Sytox red dead cell stain (Thermo Fisher) for the GFP-positive SK-BR-3 line to measure cytotoxicity. Cells were plated onto a flat-bottomed 96 well plate (10,000 cells per well, 95 uL), then 5 uL of AbLec, Siglec, or antibody in PBS was added and mixed, followed by centrifugation at 30×g for 1 min. Images were acquired every 2 h for 3 days. SK-BR-3 cells were analyzed by phase with segmentation adjustment=1, and a minimum area of 200 μm², cell death was quantified by red fluorescence using a threshold of 0.3 RCU, with an edge sensitivity of −50 and areas between 50 and 1000 square microns. HCC-1954 cells had a 0.9 segmentation adjustment, 300 square micron hole-fill, and a minimum area of 200 sq. microns. Sytox green death events were detected with a threshold of 1, an edge sensitivity of −45, and areas between 100-3000 square microns. K562 phase segmentation adjustment was 0.2, with no hole-fill and a minimum area of 60 microns. In the green fluorescence channel, the threshold was 2, with an edge sensitivity of −45 and areas between 50 and 800 microns with eccentricity and integrated intensities less than 0.95 and 40000, respectively.

### Statistical Analysis

Statistical analysis was performed in Prism (version 9). For binding curves, one-site-specific binding curves were used to calculate the dissociation constants of antibodies and AbLecs. In binding assays, NK cell cytotoxicity experiments, phagocytosis experiments, and Siglec expression analyses, ordinary one-way ANOVAs were performed with Tukey's multiple comparison's test to compare treatment groups. In every instance the asterisk * indicates a p<0.05, ** indicates p<0.01, *** indicates p<0.001, and **** indicates p<0.0001.

### Amino Acid Sequences

The amino acid sequences of the AbLecs and antibodies employed herein are show in the table below. Italicized amino acids represent signal export sequences that are not present in the final purified molecule. HA-tags and hexahistidine tags are underlined or bolded, and stop codons are represented with an asterix *.

| Protein name | Amino acid sequence |
|---|---|
| Trastuzumab- | MGWSLILLFLVAVATRVHSEVQLVESGGGLVQPGGSLRLSCAASGFNI |
| KNOB heavy | KDTYIHWVRQAPGKGLEWVARIYPTNGYTRYADSVKGRFTISADTSKN |
| chain: SEQ ID | TAYLQMNSLRAEDTAVYYCSRWGGDGFYAMDYWGQGTLVTVSSAST |
| NO: 1 | KGPSVFPLAPSSKSTSGGTAALGCLVKDYFPEPVTVSWNSGALTSGVH |
| Trastuzumab- | TFPAVLQSSGLYSLSSVVTVPSSSLGTQTYICNVNHKPSNTKVDKKVEP |
| KNOB heavy | KSCDKTHTCPPCPAPELLGGPSVFLFPPKPKDTLMISRTPEVTCVVVDV |
| chain (no signal | SHEDPEVKFNWYVDGVEVHNAKTKPREEQYNSTYRVVSVLTVLHQDW |
| export sequence | LNGKEYKCKVSNKALPAPIEKTISKAKGQPREPQVYTLPPCRDELTKNQ |
| or tag): SEQ ID | VSLWCLVKGFYPSDIAVEWESNGQPENNYKTTPPVLDSDGSFFLYSKL |
| NO: 2 | TVDKSRWQQGNVFSCSVMHEALHNHYTQKSLSLSPGGHHHHHH* |
| Trastuzumab light | MRVPAQLLGLLLLWLPGARCDIQMTQSPSSLSASVGDRVTITCRASQD |
| chain: | VNTAVAWYQQKPGKAPKLLIYSASFLYSGVPSRFSGSRSGTDFTLTISS |
| SEQ ID NO: 3 | LQPEDFATYYCQQHYTTPPTFGQGTKVEIKRTVAAPSVFIFPPSDEQLK |
| Trastuzumab light | SGTASVVCLLNNFYPREAKVQWKVDNALQSGNSQESVTEQDSKDSTY |
| chain (no signal | SLSSTLTLSKADYEKHKVYACEVTHQGLSSPVTKSFNRGEC* |
| export | |
| sequence): SEQ | |
| ID NO: 4 | |
| Rituximab-KNOB | MGWSCIILFLVATATGVHSQVQLQQPGAELVKPGASVKMSCKASGYTF |
| heavy chain: | TSYNMHWVKQTPGRGLEWIGAIYPGNGDTSYNQKFKGKATLTADKSS |
| SEQ ID NO: 5 | STAYMQLSSLTSEDSAVYYCARSTYYGGDWYFNVWGAGTTVTVSAAS |
| Rituximab-KNOB | TKGPSVFPLAPSSKSTSGGTAALGCLVKDYFPEPVTVSWNSGALTSGV |
| heavy chain (no | HTFPAVLQSSGLYSLSSVVTVPSSSLGTQTYICNVNHKPSNTKVDKKAE |
| signal export | PKSCDKTHTCPPCPAPELLGGPSVFLFPPKPKDTLMISRTPEVTCVVVD |
| sequence or tag): | VSHEDPEVKFNWYVDGVEVHNAKTKPREEQYNSTYRVVSVLTVLHQD |
| SEQ ID NO: 6 | WLNGKEYKCKVSNKALPAPIEKTISKAKGQPREPQVYTLPPCRDELTKN |
| | QVSLWCLVKGFYPSDIAVEWESNGQPENNYKTTPPVLDSDGSFFLYSK |
| | LTVDKSRWQQGNVFSCSVMHEALHNHYTQKSLSLSPGGHHHHHH* |
| Rituximab light | MGWSCIILFLVATATGVHSQIVLSQSPAILSASPGEKVTMTCRASSSVSY |
| chain: | IHWFQQKPGSSPKPWIYATSNLASGVPVRFSGSGSGTSYSLTISRVEA |
| SEQ ID NO: 7 | EDAATYYCQQWTSNPPTFGGGTKLEIKRTVAAPSVFIFPPSDEQLKSGT |
| Rituximab light | ASVVCLLNNFYPREAKVQWKVDNALQSGNSQESVTEQDSKDSTYSLS |
| chain (no signal | STLTLSKADYEKHKVYACEVTHQGLSSPVTKSFNRGEC* |
| export | |
| sequence): SEQ | |
| ID NO: 8 | |
| Cetuximab KNOB | MGWSCIILFLVATATGVHSQVQLKQSGPGLVQPSQSLSITCTVSGFSLT |
| Heavy chain: | NYGVHWVRQSPGKGLEWLGVIWSGGNTDYNTPFTSRLSINKDNSKSQ |
| SEQ ID NO: 9 | VFFKMNSLQSNDTAIYYCARALTYYDYEFAYWGQGTLVTVSAASTKGP |
| Cetuximab KNOB | SVFPLAPSSKSTSGGTAALGCLVKDYFPEPVTVSWNSGALTSGVHTFP |
| Heavy chain (no | AVLQSSGLYSLSSVVTVPSSSLGTQTYICNVNHKPSNTKVDKKVEPKSC |
| signal export | DKTHTCPPCPAPELLGGPSVFLFPPKPKDTLMISRTPEVTCVVVDVSHE |
| sequence or tag): | DPEVKFNWYVDGVEVHNAKTKPREEQYNSTYRVVSVLTVLHQDWLNG |
| SEQ ID NO: 10 | KEYKCKVSNKALPAPIEKTISKAKGQPREPQVYTLPPCRDELTKNQVSL |
| | WCLVKGFYPSDIAVEWESNGQPENNYKTTPPVLDSDGSFFLYSKLTVD |
| | KSRWQQGNVFSCSVMHEALHNHYTQKSLSLSPGGHHHHHH* |
| Cetuximab light | MGWSCIILFLVATATGVHSDILLTQSPVILSVSPGERVSFSCRASQSIGT |
| chain: | NIHWYQQRTNGSPRLLIKYASESISGIPSRFSGSGSGTDFTLSINSVESE |
| SEQ ID NO: 11 | DIADYYCQQNNNWPTTFGAGTKLELKRTVAAPSVFIFPPSDEQLKSGTA |
| Cetuximab light | SVVCLLNNFYPREAKVQWKVDNALQSGNSQESVTEQDSKDSTYSLSS |
| chain (no signal | TLTLSKADYEKHKVYACEVTHQGLSSPVTKSFNRGEC* |
| export | |
| sequence): SEQ | |
| ID NO: 12 | |
| Siglec-7 HOLES- | MLLLLLLPLLWGRERVEGQKSNRKDYSLTMQSSVTVQEGMCVHVRCS |
| Fc: | FSYPVDSQTDSDPVHGYWFRAGNDISWKAPVATNNPAWAVQEETRD |
| SEQ ID NO: 13 | RFHLLGDPQTKNCTLSIRDARMSDAGRYFFRMEKGNIKWNYKYDQLSV |
| Siglec-7 HOLES- | NVTALTHRPNILIPGTLESGCFQNLTCSVPWACEQGTPPMISWMGTSV |
| Fc (no signal | SPLHPSTTRSSVLTLIPQPQHHGTSLTCQVTLPGAGVTTNRTIQLNVSY |
| export sequence | PPQNLTVTVFQGEGTASTALGNSSSLSVLEGQSLRLVCAVDSNPPARL |
| or tag): | SWTWRSLTLYPSQPSNPLVLELQVHLGDEGEFTCRAQNSLGSQHVSL |
| SEQ ID NO: 14 | NLSLQQEYTGKMRPVSGGGGGGPKSCDKTHTCPPCPAPELLGGPSVF |
| Siglec-7 HOLES- | LFPPKPKDTLMISRTPEVTCVVVDVSHEDPEVKFNWYVDGVEVHNAKT |
| Fc Binding | KPREEQYNSTYRVVSVLTVLHQDWLNGKEYKCKVSNKALPAPIEKTISK |
| Domain: | AKGQPREPQVCTLPPSRDELTKNQVSLSCAVKGFYPSDIAVEWESNG |
| SEQ ID NO: 15 | QPENNYKTTPPVLDSDGSFFLVSKLTVDKSRWQQGNVFSCSVMHEAL |
| | HNHYTQKSLSLSPGGYPYDVPDYA* |
| Siglec-7 | MLLLLLLPLLWGRERVEGQKSNRKDYSLTMQSSVTVQEGMCVHVRCS |
| R124A HOLES- | FSYPVDSQTDSDPVHGYWFRAGNDISWKAPVATNNPAWAVQEETRD |
| Fc: | RFHLLGDPQTKNCTLSIRDARMSDAGRYFFAMEKGNIKWNYKYDQLSV |
| SEQ ID NO: 16 | NVTALTHRPNILIPGTLESGCFQNLTCSVPWACEQGTPPMISWMGTSV |
| Siglec-7 | SPLHPSTTRSSVLTLIPQPQHHGTSLTCQVTLPGAGVTTNRTIQLNVSY |
| R124A HOLES- | PPQNLTVTVFQGEGTASTALGNSSSLSVLEGQSLRLVCAVDSNPPARL |
| Fc (no signal | SWTWRSLTLYPSQPSNPLVLELQVHLGDEGEFTCRAQNSLGSQHVSL |
| export sequence | NLSLQQEYTGKMRPVSGGGGGPKSCDKTHTCPPCPAPELLGGPSVFL |
| or tag): | FPPKPKDTLMISRTPEVTCVVVDVSHEDPEVKFNWYVDGVEVHNAKTK |
| SEQ ID NO: 17 | PREEQYNSTYRVVSVLTVLHQDWLNGKEYKCKVSNKALPAPIEKTISKA |
| Siglec-7 | KGQPREPQVCTLPPSRDELTKNQVSLSCAVKGFYPSDIAVEWESNGQ |
| R124A HOLES- | PENNYKTTPPVLDSDGSFFLVSKLTVDKSRWQQGNVFSCSVMHEALH |
| Fc Binding | NHYTQKSLSLSPGGYPYDVPDYA* |
| Domain: | |
| SEQ ID NO: 18 | |
| Siglec-9 HOLES- | MLLLLLPLLWGRERAEGQTSKLLTMQSSVTVQEGLCVHVPCSFSYPSH |
| Fc: | GWIYPGPVVHGYWFREGANTDQDAPVATNNPARAVWEETRDRFHLL |
| SEQ ID NO: 19 | GDPHTKNCTLSIRDARRSDAGRYFFRMEKGSIKWNYKHHRLSVNVTAL |
| Siglec-9 HOLES- | THRPNILIPGTLESGCPQNLTCSVPWACEQGTPPMISWIGTSVSPLDPS |
| Fc (no signal | TTRSSVLTLIPQPQDHGTSLTCQVTFPGASVTTNKTVHLNVSYPPQNLT |
| export sequence | MTVFQGDGTVSTVLGNGSSLSLPEGQSLRLVCAVDAVDSNPPARLSLS |
| or tag): | WRGLTLCPSQPSNPGVLELPWVHLRDAAEFTCRAQNPLGSQQVYLNV |
| SEQ ID NO: 20 | SLQSKATSGGGGGPKSCDKTHTCPPCPAPELLGGPSVFLFPPKPKDTL |
| Siglec-9 HOLES- | MISRTPEVTCVVVDVSHEDPEVKFNWYVDGVEVHNAKTKPREEQYNS |
| Fc Binding | TYRVVSVLTVLHQDWLNGKEYKCKVSNKALPAPIEKTISKAKGQPREPQ |
| Domain: | VCTLPPSRDELTKNQVSLSCAVKGFYPSDIAVEWESNGQPENNYKTTP |
| SEQ ID NO: 21 | PVLDSDGSFFLVSKLTVDKSRWQQGNVFSCSVMHEALHNHYTQKSLS |
| | LSPGGYPYDVPDYA* |
| Siglec-9 | MLLLLLPLLWGRERAEGQTSKLLTMQSSVTVQEGLCVHVPCSFSYPSH |
| R120A HOLES- | GWIYPGPVVHGYWFREGANTDQDAPVATNNPARAVWEETRDRFHLL |
| Fc: | GDPHTKNCTLSIRDARRSDAGRYFFAMEKGSIKWNYKHHRLSVNVTAL |
| SEQ ID NO: 22 | THRPNILIPGTLESGCPQNLTCSVPWACEQGTPPMISWIGTSVSPLDPS |
| Siglec-9 | TTRSSVLTLIPQPQDHGTSLTCQVTFPGASVTTNKTVHLNVSYPPQNLT |
| R120A HOLES- | MTVFQGDGTVSTVLGNGSSLSLPEGQSLRLVCAVDAVDSNPPARLSLS |
| Fc (no signal | WRGLTLCPSQPSNPGVLELPWVHLRDAAEFTCRAQNPLGSQQVYLNV |
| export sequence | SLQSKATSGGGGGPKSCDKTHTCPPCPAPELLGGPSVFLFPPKPKDTL |
| or tag): SEQ ID | MISRTPEVTCVVVDVSHEDPEVKFNWYVDGVEVHNAKTKPREEQYNS |
| NO: 23 | TYRVVSVLTVLHQDWLNGKEYKCKVSNKALPAPIEKTISKAKGQPREPQ |
| Siglec-9 | VCTLPPSRDELTKNQVSLSCAVKGFYPSDIAVEWESNGQPENNYKTTP |
| R120A HOLES- | PVLDSDGSFFLVSKLTVDKSRWQQGNVFSCSVMHEALHNHYTQKSLS |
| Fc Binding | LSPGGYPYDVPDYA* |
| Domain: | |
| SEQ ID NO: 24 | |
| Siglec-10 | MLLPLLLSSLLGGSQAMDGRFWIRVQESVMVPEGLCISVPCSFSYPRQ |
| HOLES-Fc HA: | DWTGSTPAYGYWFKAVTETTKGAPVATNHQSREVEMSTRGRFQLTG |
| SEQ ID NO: 26 | DPAKGNCSLVIRDAQMQDESQYFFRVERGSYVRYNFMNDGFFLKVTA |
| Siglec-10 | LTQKPDVYIPETLEPGQPVTVICVFNWAFEECPPPSFSWTGAALSSQG |
| HOLES-Fc HA | TKPTTSHFSVLSFTPRPQDHNTDLTCHVDFSRKGVSAQRTVRLRVAYA |
| (no signal export | PRDLVISISRDNTPALEPQPQGNVPYLEAQKGQFLRLLCAADSQPPATL |
| sequence or tag): | SWVLQNRVLSSSHPWGPRPLGLELPGVKAGDSGRYTCRAENRLGSQ |
| SEQ ID NO: 27 | QRALDLSVQYPPENLRVMVSQANRTVLENLGNGTSLPVLEGQSLCLVC |
| Siglec-10 | VTHSSPPARLSWTQRGQVLSPSQPSDPGVLELPRVQVEHEGEFTCHA |
| HOLES-Fc HA | RHPLGSQHVSLSLSVHYSPKLLGPSCSWEAEGLHCSCSSQASPAPSL |
| Binding Domain: | RWWLGEELLEGNSSQDSFEVTPSSAGPWANSSLSLHGGLSSGLRLRC |
| SEQ ID NO: 28 | EAWNVHGAQSGSILQLPDKKGLISTGGGGGPKSCDKTHTCPPCPAPEL |
| | LGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALP |
| | APIEKTISKAKGQPREPQVCTLPPSRDELTKNQVSLSCAVKGFYPSDIA |
| | VEWESNGQPENNYKTTPPVLDSDGSFFLVSKLTVDKSRWQQGNVFSC |
| | SVMHEALHNHYTQKSLSLSPGGYPYDVPDYA* |
| Pembro HC | MGWSCIILFLVATATGVHSQVQLVQSGVEVKKPGASVKVSCKASGYTF |
| knobs LALA | TNYYMYWVRQAPGQGLEWMGGINPSNGGTNFNEKFKNRVTLTTDSS |
| P329G 6xHis: | TTTAYMELKSLQFDDTAVYYCARRDYRFDMGFDYWGQGTTVTVSSAS |
| SEQ ID NO: 29 | TKGPSVFPLAPSSKSTSGGTAALGCLVKDYFPEPVTVSWNSGALTSGV |
| Pembro HC | HTFPAVLQSSGLYSLSSVVTVPSSSLGTQTYICNVNHKPSNTKVDKKVE |
| knobs LALA | PKSCDKTHTCPPCPAPEAAGGPSVFLFPPKPKDTLMISRTPEVTCVVV |
| P329G (no signal | DVSHEDPEVKFNWYVDGVEVHNAKTKPREEQYNSTYRVVSVLTVLHQ |
| export sequence | DWLNGKEYKCKVSNKALGAPIEKTISKAKGQPREPQVYTLPPCRDELT |
| or tag): | KNQVSLWCLVKGFYPSDIAVEWESNGQPENNYKTTPPVLDSDGSFFLY |
| SEQ ID NO: 30 | SKLTVDKSRWQQGNVFSCSVMHEALHNHYTQKSLSLSPGGHHHHHH* |
| Pembro LC: | MGWSCIILFLVATATGVHSEIVLTQSPATLSLSPGERATLSCRASKGVST |
| SEQ ID NO: 31 | SGYSYLHWYQQKPGQAPRLLIYLASYLESGVPARFSGSGSGTDFTLTIS |
| Pembro LC (no | SLEPEDFAVYYCQHSRDLPLTFGGGTKVEIKRTVAAPSVFIFPPSDEQL |
| signal export | KSGTASVVCLLNNFYPREAKVQWKVDNALQSGNSQESVTEQDSKDST |
| sequence): | YSLSSTLTLSKADYEKHKVYACEVTHQGLSSPVTKSFNRGEC* |
| SEQ ID NO: 32 | |
| Nivolumab HC | MGWSCIILFLVATATGVHSQVQLVESGGGVVQPGRSLRLDCKASGITF |
| knobs LALA | SNSGMHWVRQAPGKGLEWVAVIWYDGSKRYYADSVKGRFTISRDNS |
| P329G 6xHis: | KNTLFLQMNSLRAEDTAVYYCATNDDYWGQGTLVTVSSASTKGPSVF |
| SEQ ID NO: 33 | PLAPSSKSTSGGTAALGCLVKDYFPEPVTVSWNSGALTSGVHTFPAVL |
| Nivolumab HC | QSSGLYSLSSVVTVPSSSLGTQTYICNVNHKPSNTKVDKKVEPKSCDK |
| knobs LALA | THTCPPCPAPEAAGGPSVFLFPPKPKDTLMISRTPEVTCVVVDVSHED |
| P329G (no signal | PEVKFNWYVDGVEVHNAKTKPREEQYNSTYRVVSVLTVLHQDWLNGK |
| export sequence | EYKCKVSNKALGAPIEKTISKAKGQPREPQVYTLPPCRDELTKNQVSLW |
| or tag): | CLVKGFYPSDIAVEWESNGQPENNYKTTPPVLDSDGSFFLYSKLTVDK |
| SEQ ID NO: 34 | SRWQQGNVFSCSVMHEALHNHYTQKSLSLSPGGHHHHHH* |
| Nivolumab LC: | MGWSCIILFLVATATGVHSEIVLTQSPATLSLSPGERATLSCRASQSVSS |
| SEQ ID NO: 35 | YLAWYQQKPGQAPRLLIYDASNRATGIPARFSGSGSGTDFTLTISSLEP |
| Nivolumab LC | EDFAVYYCQQSSNWPRTFGQGTKVEIKRTVAAPSVFIFPPSDEQLKSG |
| (no signal export | TASVVCLLNNFYPREAKVQWKVDNALQSGNSQESVTEQDSKDSTYSL |
| sequence): | SSTLTLSKADYEKHKVYACEVTHQGLSSPVTKSFNRGEC* |
| SEQ ID NO: 36 | |
| Siglec-7 Holes | MLLLLLLPLLWGRERVEGQKSNRKDYSLTMQSSVTVQEGMCVHVRCS |
| LALA P329G Fc | FSYPVDSQTDSDPVHGYWFRAGNDISWKAPVATNNPAWAVQEETRD |
| HA: | RFHLLGDPQTKNCTLSIRDARMSDAGRYFFRMEKGNIKWNYKYDQLSV |
| SEQ ID NO: 37 | NVTALTHRPNILIPGTLESGCFQNLTCSVPWACEQGTPPMISWMGTSV |
| Siglec-7 Holes | SPLHPSTTRSSVLTLIPQPQHHGTSLTCQVTLPGAGVTTNRTIQLNVSY |
| LALA P329G Fc | PPQNLTVTVFQGEGTASTALGNSSSLSVLEGQSLRLVCAVDSNPPARL |
| HA (no signal | SWTWRSLTLYPSQPSNPLVLELQVHLGDEGEFTCRAQNSLGSQHVSL |
| export sequence | NLSLQQEYTGKMRPVSGGGGGGPKSCDKTHTCPPCPAPEAAGGPSV |
| or tag): | FLFPPKPKDTLMISRTPEVTCVVVDVSHEDPEVKFNWYVDGVEVHNAK |
| SEQ ID NO: 38 | TKPREEQYNSTYRVVSVLTVLHQDWLNGKEYKCKVSNKALGAPIEKTIS |
| Siglec-7 Holes | KAKGQPREPQVCTLPPSRDELTKNQVSLSCAVKGFYPSDIAVEWESNG |
| LALA P329G Fc | QPENNYKTTPPVLDSDGSFFLVSKLTVDKSRWQQGNVFSCSVMHEAL |
| HA Binding | HNHYTQKSLSLSPGGYPYDVPDYA* |
| Domain: | |
| SEQ ID NO: 15 | |
| Siglec-9 Holes | MLLLLLPLLWGRERAEGQTSKLLTMQSSVTVQEGLCVHVPCSFSYPSH |
| LALA P329G Fc | GWIYPGPVVHGYWFREGANTDQDAPVATNNPARAVWEETRDRFHLL |
| HA: | GDPHTKNCTLSIRDARRSDAGRYFFRMEKGSIKWNYKHHRLSVNVTAL |
| SEQ ID NO: 39 | THRPNILIPGTLESGCPQNLTCSVPWACEQGTPPMISWIGTSVSPLDPS |
| Siglec-9 Holes | TTRSSVLTLIPQPQDHGTSLTCQVTFPGASVTTNKTVHLNVSYPPQNLT |
| LALA P329G Fc | MTVFQGDGTVSTVLGNGSSLSLPEGQSLRLVCAVDAVDSNPPARLSLS |
| HA (no signal | WRGLTLCPSQPSNPGVLELPWVHLRDAAEFTCRAQNPLGSQQVYLNV |
| export sequence | SLQSKATSGGGGGPKSCDKTHTCPPCPAPEAAGGPSVFLFPPKPKDT |
| or tag): | LMISRTPEVTCVVVDVSHEDPEVKFNWYVDGVEVHNAKTKPREEQYN |
| SEQ ID NO: 40 | STYRVVSVLTVLHQDWLNGKEYKCKVSNKALGAPIEKTISKAKGQPREP |
| Siglec-9 Holes | QVCTLPPSRDELTKNQVSLSCAVKGFYPSDIAVEWESNGQPENNYKTT |
| LALA P329G Fc | PPVLDSDGSFFLVSKLTVDKSRWQQGNVFSCSVMHEALHNHYTQKSL |
| HA Binding | SLSPGGYPYDVPDYA* |
| Domain: | |
| SEQ ID NO: 21 | |
| Siglec-10Ig2 | MLLPLLLSSLLGGSQAMDGRFWIRVQESVMVPEGLCISVPCSFSYPRQ |
| Holes Fc HA: | DWTGSTPAYGYWFKAVTETTKGAPVATNHQSREVEMSTRGRFQLTG |
| SEQ ID NO: 41 | DPAKGNCSLVIRDAQMQDESQYFFRVERGSYVRYNFMNDGFFLKVTA |
| Siglec-10Ig2 | LTQKPDVYIPETLEPGQPVTVICVFNWAFEECPPPSFSWTGAALSSQG |
| Holes Fc HA (no | TKPTTSHFSVLSFTPRPQDHNTDLTCHVDFSRKGVSAQRTVRLRVAYA |
| signal export | PRDLVISISRDNTPALEPQPQGNVPYLEAQKGQFLRLLCAADSQPPATL |
| sequence or tag): | SWVLQNRVLSSSHPWGPRPLGLELPGVKAGDSGRYTCRAENRLGSQ |
| SEQ ID NO: 42 | QRALDLSGGGGGPKSCDKTHTCPPCPAPELLGGPSVFLFPPKPKDTL |
| Siglec-10Ig2 | MISRTPEVTCVVVDVSHEDPEVKFNWYVDGVEVHNAKTKPREEQYNS |
| Holes Fc HA | TYRVVSVLTVLHQDWLNGKEYKCKVSNKALPAPIEKTISKAKGQPREPQ |
| Binding Domain: | VCTLPPSRDELTKNQVSLSCAVKGFYPSDIAVEWESNGQPENNYKTTP |
| SEQ ID NO: 43 | PVLDSDGSFFLVSKLTVDKSRWQQGNVFSCSVMHEALHNHYTQKSLS |
| | LSPGGYPYDVPDYA* |
| Galectin-9N | MAFSGSQAPYLSPAVPFSGTIQGGLQDGLQITVNGTVLSSSGTRFAVN |
| LALAP329G | FQTGFSGNDIAFHFNPRFEDGGYVVCNTRQNGSWGPEERKTHMPFQ |
| Holes Fc HA: | KGMPFDLCFLVQSSDFKVMVNGILFVQYFHRVPFHRVDTISVNGSVQL |
| SEQ ID NO: 44 | SYISFQGGGGGPKSCDKTHTCPPCPAPEAAGGPSVFLFPPKPKDTLMI |
| Galectin-9N | SRTPEVTCVVVDVSHEDPEVKFNWYVDGVEVHNAKTKPREEQYNSTY |
| LALAP329G | RVVSVLTVLHQDWLNGKEYKCKVSNKALGAPIEKTISKAKGQPREPQV |
| Holes Fc HA (no | CTLPPSRDELTKNQVSLSCAVKGFYPSDIAVEWESNGQPENNYKTTPP |
| signal export | VLDSDGSFFLVSKLTVDKSRWQQGNVFSCSVMHEALHNHYTQKSLSL |
| sequence or tag): | SPGGYPYDVPDYA* |
| SEQ ID NO: 45 | |
| Galectin-9N | |
| LALAP329G | |
| Holes Fc HA | |
| Binding Domain: | |
| SEQ ID NO: 46 | |
| Ritux HC knobs | MGWSCIILFLVATATGVHSQVQLQQPGAELVKPGASVKMSCKASGYTF |
| LALA P329G | TSYNMHWVKQTPGRGLEWIGAIYPGNGDTSYNQKFKGKATLTADKSS |
| 6xHis: | STAYMQLSSLTSEDSAVYYCARSTYYGGDWYFNVWGAGTTVTVSAAS |
| SEQ ID NO: 47 | TKGPSVFPLAPSSKSTSGGTAALGCLVKDYFPEPVTVSWNSGALTSGV |
| Ritux HC knobs | HTFPAVLQSSGLYSLSSVVTVPSSSLGTQTYICNVNHKPSNTKVDKKAE |
| LALA P329G (no | PKSCDKTHTCPPCPAPEAAGGPSVFLFPPKPKDTLMISRTPEVTCVVV |
| signal export | DVSHEDPEVKFNWYVDGVEVHNAKTKPREEQYNSTYRVVSVLTVLHQ |
| sequence or tag): | DWLNGKEYKCKVSNKALGAPIEKTISKAKGQPREPQVYTLPPCRDELT |
| SEQ ID NO: 48 | KNQVSLWCLVKGFYPSDIAVEWESNGQPENNYKTTPPVLDSDGSFFLY |
| | SKLTVDKSRWQQGNVFSCSVMHEALHNHYTQKSLSLSPGGHHHHHH* |
| Tafasitamab HC | MGWSCIILFLVATATGVHSEVQLVESGGGLVKPGGSLKLSCAASGYTFT |
| knobs LALA | SYVMHWVRQAPGKGLEWIGYINPYNDGTKYNEKFQGRVTISSDKSIST |
| P329G 6xHis: | AYMELSSLRSEDTAMYYCARGTYYYGTRVFDYWGQGTLVTVSSASTK |
| SEQ ID NO: 49 | GPSVFPLAPSSKSTSGGTAALGCLVKDYFPEPVTVSWNSGALTSGVHT |
| Tafastamab HC | FPAVLQSSGLYSLSSVVTVPSSSLGTQTYICNVNHKPSNTKVDKKVEPK |
| knobs LALA | SCDKTHTCPPCPAPEAAGGPSVFLFPPKPKDTLMISRTPEVTCVVVDV |
| P329G (no signal | SHEDPEVKFNWYVDGVEVHNAKTKPREEQYNSTYRVVSVLTVLHQDW |
| export sequence | LNGKEYKCKVSNKALGAPIEKTISKAKGQPREPQVYTLPPCRDELTKNQ |
| or tag): | VSLWCLVKGFYPSDIAVEWESNGQPENNYKTTPPVLDSDGSFFLYSKL |
| SEQ ID NO: 50 | TVDKSRWQQGNVFSCSVMHEALHNHYTQKSLSLSPGGHHHHHH* |
| Tafasitamab LC: | MGWSCIILFLVATATGVHSDIVMTQSPATLSLSPGERATLSCRSSKSLQ |
| SEQ ID NO: 51 | NVNGNTYLYWFQQKPGQSPQLLIYRMSNLNSGVPDRFSGSGSGTEFT |
| Tafasitamab LC | LTISSLEPEDFAVYYCMQHLEYPITFGAGTKLEIKRTVAAPSVFIFPPSDE |
| (no signal export | QLKSGTASVVCLLNNFYPREAKVQWKVDNALQSGNSQESVTEQDSKD |
| sequence): | STYSLSSTLTLSKADYEKHKVYACEVTHQGLSSPVTKSFNRGEC* |
| SEQ ID NO: 52 | |
| HuB6H12 HC | MGWSCIILFLVATATGVHSEVQLVESGGGLVQPGGSLRLSCAASGFTF |
| knobs LALA | SGYGMSWVRQAPGKGLEWVATITSGGTYTYYPDSVKGRFTISRDNAK |
| P329G 6xHis: | NSLYLQMNSLRAEDTAVYYCARSLAGNAMDYWGQGTLVTVSSASTKG |
| SEQ ID NO: 53 | PSVFPLAPSSKSTSGGTAALGCLVKDYFPEPVTVSWNSGALTSGVHTF |
| HuB6H12 HC | PAVLQSSGLYSLSSVVTVPSSSLGTQTYICNVNHKPSNTKVDKKVEPKS |
| knobs LALA | CDKTHTCPPCPAPEAAGGPSVFLFPPKPKDTLMISRTPEVTCVVVDVS |
| P329G (no signal | HEDPEVKFNWYVDGVEVHNAKTKPREEQYNSTYRVVSVLTVLHQDWL |
| export sequence | NGKEYKCKVSNKALGAPIEKTISKAKGQPREPQVYTLPPCRDELTKNQV |
| or tag): | SLWCLVKGFYPSDIAVEWESNGQPENNYKTTPPVLDSDGSFFLYSKLT |
| SEQ ID NO: 54 | VDKSRWQQGNVFSCSVMHEALHNHYTQKSLSLSPGGHHHHHH* |
| HuB6H12 LC: | MGWSCIILFLVATATGVHSEIVLTQSPATLSLSPGERATLSCRASQTISD |
| SEQ ID NO: 55 | YLHWYQQKPGQAPRLLIKFASQSISGIPARFSGSGSGTDFTLTISSLEPE |
| HuB6H12 LC: 56 | DFAVYYCQNGHGFPRTFGQGTKVEIKRTVAAPSVFIFPPSDEQLKSGT |
| | ASVVCLLNNFYPREAKVQWKVDNALQSGNSQESVTEQDSKDSTYSLS |
| | STLTLSKADYEKHKVYACEVTHQGLSSPVTKSFNRGEC* |
| Hu5F9 HC knobs | MGWSCIILFLVATATGVHSQVQLQQPGAELVKPGASVMMSCKASGYTF |
| LALA P329G | TNYNMHWVKQTPGQGLEWIGTIYPGNDDTSYNQKFKDKATLTADKSS |
| 6xHis: | SAAYMQLSSLTSEDSAVYYCARGGYRAMDYWGQGTSVTVSSASTKG |
| SEQ ID NO: 57 | PSVFPLAPSSKSTSGGTAALGCLVKDYFPEPVTVSWNSGALTSGVHTF |
| Hu5F9 HC knobs | PAVLQSSGLYSLSSVVTVPSSSLGTQTYICNVNHKPSNTKVDKKVEPKS |
| LALA P329G (no | CDKTHTCPPCPAPEAAGGPSVFLFPPKPKDTLMISRTPEVTCVVVDVS |
| signal export | HEDPEVKFNWYVDGVEVHNAKTKPREEQYNSTYRVVSVLTVLHQDWL |
| sequence or tag): | NGKEYKCKVSNKALGAPIEKTISKAKGQPREPQVYTLPPCRDELTKNQV |
| SEQ ID NO: 58 | SLWCLVKGFYPSDIAVEWESNGQPENNYKTTPPVLDSDGSFFLYSKLT |
| | VDKSRWQQGNVFSCSVMHEALHNHYTQKSLSLSPGGHHHHHH* |
| Hu5F9 LC: | MGWSCIILFLVATATGVHSDVLMTQTPLSLPVSLGDQASISCRSSQSIVY |
| SEQ ID NO: 59 | SNGNTYLGWYLQKPGQSPKLLIYKVSNRFSGVPDRFSGSGSGTDFTLK |
| Hu5F9 LC (no | ISRVEAEDLGVYHCFQGSHVPYTFGGGTKVEIKRTVAAPSVFIFPPSDE |
| signal export | QLKSGTASVVCLLNNFYPREAKVQWKVDNALQSGNSQESVTEQDSKD |
| sequence): | STYSLSSTLTLSKADYEKHKVYACEVTHQGLSSPVTKSFNRGEC* |
| SEQ ID NO: 60 | |
| Avelumab HC | MGWSCIILFLVATATGVHSEVQLLESGGGLVQPGGSLRLSCAASGFTF |
| knobs 6xHis: | SSYIMMWVRQAPGKGLEWVSSIYPSGGITFYADTVKGRFTISRDNSKN |
| SEQ ID NO: 61 | TLYLQMNSLRAEDTAVYYCARIKLGTVTTVDYWGQGTLVTVSSASTKG |
| Avelumab HC | PSVFPLAPSSKSTSGGTAALGCLVKDYFPEPVTVSWNSGALTSGVHTF |
| knobs (no signal | PAVLQSSGLYSLSSVVTVPSSSLGTQTYICNVNHKPSNTKVDKKVEPKS |
| export sequence | CDKTHTCPPCPAPELLGGPSVFLFPPKPKDTLMISRTPEVTCVVVDVSH |
| or tag): SEQ ID | EDPEVKFNWYVDGVEVHNAKTKPREEQYNSTYRVVSVLTVLHQDWLN |
| NO: 62 | GKEYKCKVSNKALPAPIEKTISKAKGQPREPQVYTLPPCRDELTKNQVS |
| | LWCLVKGFYPSDIAVEWESNGQPENNYKTTPPVLDSDGSFFLYSKLTV |
| | DKSRWQQGNVFSCSVMHEALHNHYTQKSLSLSPGGHHHHHH* |
| Avelumab LC: | MGWSCIILFLVATATGVHSQSALTQPASVSGSPGQSITISCTGTSSDVG |
| SEQ ID NO: 63 | GYNYVSWYQQHPGKAPKLMIYDVSNRPSGVSNRFSGSKSGNTASLTIS |
| Avelumab LC (no | GLQAEDEADYYCSSYTSSSTRVFGTGTKVTVLGQPKANPTVTLFPPSS |
| signal export | EELQANKATLVCLISDFYPGAVTVAWKADGSPVKAGVETTKPSKQSNN |
| sequence): | KYAASSYLSLTPEQWKSHRSYSCQVTHEGSTVEKTVAPTEC* |
| SEQ ID NO: 64 | |
| Atezolizumab HC | MGWSCIILFLVATATGVHSEVQLVESGGGLVQPGGSLRLSCAASGFTF |
| knobs 6xHis: | SDSWIHWVRQAPGKGLEWVAWISPYGGSTYYADSVKGRFTISADTSK |
| SEQ ID NO: 65 | NTAYLQMNSLRAEDTAVYYCARRHWPGGFDYWGQGTLVTVSSASTK |
| Atezolizumab HC | GPSVFPLAPSSKSTSGGTAALGCLVKDYFPEPVTVSWNSGALTSGVHT |
| knobs (no signal | FPAVLQSSGLYSLSSVVTVPSSSLGTQTYICNVNHKPSNTKVDKKVEPK |
| export sequence | SCDKTHTCPPCPAPEAAGGPSVFLFPPKPKDTLMISRTPEVTCVVVDV |
| or tag): | SHEDPEVKFNWYVDGVEVHNAKTKPREEQYNSTYRVVSVLTVLHQDW |
| SEQ ID NO: 66 | LNGKEYKCKVSNKALGAPIEKTISKAKGQPREPQVYTLPPCRDELTKNQ |
| | VSLWCLVKGFYPSDIAVEWESNGQPENNYKTTPPVLDSDGSFFLYSKL |
| | TVDKSRWQQGNVFSCSVMHEALHNHYTQKSLSLSPGGHHHHHH* |
| Atezolizumab LC: | MGWSCIILFLVATATGVHSDIQMTQSPSSLSASVGDRVTITCRASQDVS |
| SEQ ID NO: 67 | TAVAWYQQKPGKAPKLLIYSASFLYSGVPSRFSGSGSGTDFTLTISSLQ |
| Atezolizumab LC | PEDFATYYCQQYLYHPATFGQGTKVEIKRTVAAPSVFIFPPSDEQLKSG |
| (no signal export | TASVVCLLNNFYPREAKVQWKVDNALQSGNSQESVTEQDSKDSTYSL |
| sequence): | SSTLTLSKADYEKHKVYACEVTHQGLSSPVTKSFNRGEC* |
| SEQ ID NO: 68 | |
| Siglec-2FL Holes | MHLLGPWLLLLVLEYLAFSDSSKWVFEHPETLYAWEGACVWIPCTYRA |
| LALA P329G Fc | LDGDLESFILFHNPEYNKNTSKFDGTRLYESTKDGKVPSEQKRVQFLG |
| HA: | DKNKNCTLSIHPVHLNDSGQLGLRMESKTEKWMERIHLNVSERPFPPHI |
| SEQ ID NO: 69 | QLPPEIQESQEVTLTCLLNFSCYGYPIQLQWLLEGVPMRQAAVTSTSLTI |
| Siglec-2FL Holes | KSVFTRSELKFSPQWSHHGKIVTCQLQDADGKFLSNDTVQLNVKHTPK |
| LALA P329G Fc | LEIKVTPSDAIVREGDSVTMTCEVSSSNPEYTTVSWLKDGTSLKKQNTF |
| HA (no signal | TLNLREVTKDQSGKYCCQVSNDVGPGRSEEVFLQVQYAPEPSTVQILH |
| export sequence | SPAVEGSQVEFLCMSLANPLPTNYTWYHNGKEMQGRTEEKVHIPKILP |
| or tag): | WHAGTYSCVAENILGTGQRGPGAELDVQYPPKKVTTVIQNPMPIREGD |
| SEQ ID NO: 70 | TVTLSCNYNSSNPSVTRYEWKPHGAWEEPSLGVLKIQNVGWDNTTIAC |
| Siglec-2FL Holes | ARCNSWCSWASPVALNVQYAPRDVRVRKIKPLSEIHSGNSVSLQCDFS |
| LALA P329G Fc | SSHPKEVQFFWEKNGRLLGKESQLNFDSISPEDAGSYSCWVNNSIGQT |
| HA Binding | ASKAWTLEVLYAPRRLRVSMSPGDQVMEGKSATLTCESDANPPVSHY |
| Domain: | TWFDWNNQSLPYHSQKLRLEPVKVQHSGAYWCQGTNSVGKGRSPLS |
| SEQ ID NO: 71 | TLTVYYSPETIGRRGGGGGPKSCDKTHTCPPCPAPEAAGGPSVFLFPP |
| | KPKDTLMISRTPEVTCVVVDVSHEDPEVKFNWYVDGVEVHNAKTKPRE |
| | EQYNSTYRVVSVLTVLHQDWLNGKEYKCKVSNKALGAPIEKTISKAKG |
| | QPREPQVCTLPPSRDELTKNQVSLSCAVKGFYPSDIAVEWESNGQPE |
| | NNYKTTPPVLDSDGSFFLVSKLTVDKSRWQQGNVFSCSVMHEALHNH |
| | YTQKSLSLSPGGYPYDVPDYA* |
| Siglec-2Ig2 Holes | MHLLGPWLLLLVLEYLAFSDSSKWVFEHPETLYAWEGACVWIPCTYRA |
| LALA P329G Fc | LDGDLESFILFHNPEYNKNTSKFDGTRLYESTKDGKVPSEQKRVQFLG |
| HA: | DKNKNCTLSIHPVHLNDSGQLGLRMESKTEKWMERIHLNVSERPFPPHI |
| SEQ ID NO: 72 | QLPPEIQESQEVTLTCLLNFSCYGYPIQLQWLLEGVPMRQAAVTSTSLTI |
| Siglec-2Ig2 Holes | KSVFTRSELKFSPQWSHHGKIVTCQLQDADGKFLSNDTVQLNVKHTPK |
| LALA P329G Fc | LEIKVTPSDAIVREGDSVTMTCEVSSSNPEYTTVSWLKDGTSLKKQNTE |
| HA (no signal | TLNLREVTKDQSGKYCCQVSNDVGPGRSEEVFLQGGGGGPKSCDKT |
| export sequence | HTCPPCPAPEAAGGPSVFLFPPKPKDTLMISRTPEVTCVVVDVSHEDP |
| or tag): | EVKFNWYVDGVEVHNAKTKPREEQYNSTYRVVSVLTVLHQDWLNGKE |
| SEQ ID NO: 73 | YKCKVSNKALGAPIEKTISKAKGQPREPQVCTLPPSRDELTKNQVSLSC |
| Siglec-2Ig2 Holes | AVKGFYPSDIAVEWESNGQPENNYKTTPPVLDSDGSFFLVSKLTVDKS |
| LALA P329G Fc | RWQQGNVFSCSVMHEALHNHYTQKSLSLSPGGYPYDVPDYA* |
| HA Binding | |
| Domain: | |
| SEQ ID NO: 74 | |
| Siglec-10Ig2 | MLLPLLLSSLLGGSQAMDGRFWIRVQESVMVPEGLCISVPCSFSYPRQ |
| Holes LALA | DWTGSTPAYGYWFKAVTETTKGAPVATNHQSREVEMSTRGRFQLTG |
| P329G Fc HA: | DPAKGNCSLVIRDAQMQDESQYFFRVERGSYVRYNFMNDGFFLKVTA |
| SEQ ID NO: 75 | LTQKPDVYIPETLEPGQPVTVICVFNWAFEECPPPSFSWTGAALSSQG |
| Siglec-10Ig2 | TKPTTSHFSVLSFTPRPQDHNTDLTCHVDFSRKGVSAQRTVRLRVAYA |
| Holes LALA | PRDLVISISRDNTPALEPQPQGNVPYLEAQKGQFLRLLCAADSQPPATL |
| P329G Fc HA (no | SWVLQNRVLSSSHPWGPRPLGLELPGVKAGDSGRYTCRAENRLGSQ |
| signal export | QRALDLSGGGGGPKSCDKTHTCPPCPAPEAAGGPSVFLFPPKPKDTL |
| sequence | MISRTPEVTCVVVDVSHEDPEVKFNWYVDGVEVHNAKTKPREEQYNS |
| or tag): | TYRVVSVLTVLHQDWLNGKEYKCKVSNKALGAPIEKTISKAKGQPREP |
| SEQ ID NO: 76 | QVCTLPPSRDELTKNQVSLSCAVKGFYPSDIAVEWESNGQPENNYKTT |
| Siglec-10Ig2 | PPVLDSDGSFFLVSKLTVDKSRWQQGNVFSCSVMHEALHNHYTQKSL |
| Holes LALA | SLSPGGYPYDVPDYA* |
| P329G Fc HA | |
| Binding Domain: | |
| SEQ ID NO: 43 | |
| ESelSushi2 | MIASQFLSALTLVLLIKESGAWSYNTSTEAMTYDEASAYCQQRYTHLVAI |
| LALAP329G | QNKEEIEYLNSILSYSPSYYWIGIRKVNNVWVWVGTQKPLTEEAKNWA |
| Holes Fc HA: | PGEPNNRQKDEDCVEIYIKREKDVGMWNDERCSKKKLALCYTAACTNT |
| SEQ ID NO: 77 | SCSGHGECVETINNYTCKCDPGFSGLKCEQIVNCTALESPEHGSLVCS |
| ESelSushi2 | HPLGNFSYNSSCSISCDRGYLPSSMETMQCMSSGEWSAPIPACNVVE |
| LALAP329G | CDAVTNPANGFVECFQNPGSFPWNTTCTFDCEEGFELMGAQSLQCTS |
| Holes Fc HA (no | SGNWDNEKPTCKAGGGGGPKSCDKTHTCPPCPAPEAAGGPSVFLFP |
| signal export | PKPKDTLMISRTPEVTCVVVDVSHEDPEVKFNWYVDGVEVHNAKTKPR |
| sequence or tag): | EEQYNSTYRVVSVLTVLHQDWLNGKEYKCKVSNKALGAPIEKTISKAKG |
| SEQ ID NO: 78 | QPREPQVCTLPPSRDELTKNQVSLSCAVKGFYPSDIAVEWESNGQPE |
| ESelSushi2 | NNYKTTPPVLDSDGSFFLVSKLTVDKSRWQQGNVFSCSVMHEALHNH |
| LALAP329G | YTQKSLSLSPGGYPYDVPDYA* |
| Holes Fc HA: | |
| SEQ ID NO: 79 | |
| PSelSushi2 | MANCQIAILYQRFQRVVFGISQLLCFSALISELTNQKEVAAWTYHYSTKA |
| LALAP329G | YSWNISRKYCQNRYTDLVAIQNKNEIDYLNKVLPYYSSYYWIGIRKNNK |
| Holes Fc HA: | TWTWVGTKKALTNEAENWADNEPNNKRNNEDCVEIYIKSPSAPGKWN |
| SEQ ID NO: 80 | DEHCLKKKHALCYTASCQDMSCSKQGECLETIGNYTCSCYPGFYGPE |
| PSelSushi2 | CEYVRECGELELPQHVLMNCSHPLGNFSFNSQCSFHCTDGYQVNGPS |
| LALAP329G | KLECLASGIWTNKPPQCLAAQCPPLKIPERGNMTCLHSAKAFQHQSSC |
| Holes Fc HA (no | SFSCEEGFALVGPEVVQCTASGVWTAPAPVCKAGGGGGPKSCDKTHT |
| signal export | CPPCPAPEAAGGPSVFLFPPKPKDTLMISRTPEVTCVVVDVSHEDPEV |
| sequence or tag): | KFNWYVDGVEVHNAKTKPREEQYNSTYRVVSVLTVLHQDWLNGKEYK |
| SEQ ID NO: 81 | CKVSNKALGAPIEKTISKAKGQPREPQVCTLPPSRDELTKNQVSLSCAV |
| PSelSushi2 | KGFYPSDIAVEWESNGQPENNYKTTPPVLDSDGSFFLVSKLTVDKSRW |
| LALAP329G | QQGNVFSCSVMHEALHNHYTQKSLSLSPGGYPYDVPDYA* |
| Holes Fc HA | |
| Binding Domain: | |
| SEQ ID NO: 82 | |
| Teplizumab HC | MGWSCIILFLVATATGVHSQVQLVQSGGGVVQPGRSLRLSCKASGYTF |
| LALA knobs | TRYTMHWVRQAPGKGLEWIGYINPSRGYTNYNQKVKDRFTISRDNSK |
| 6xHis: | NTAFLQMDSLRPEDTGVYFCARYYDDHYCLDYWGQGTPVTVSSASTK |
| SEQ ID NO: 83 | GPSVFPLAPSSKSTSGGTAALGCLVKDYFPEPVTVSWNSGALTSGVHT |
| Teplizumab HC | FPAVLQSSGLYSLSSVVTVPSSSLGTQTYICNVNHKPSNTKVDKKVEPK |
| LALA knobs (no | SCDKTHTCPPCPAPEAAGGPSVFLFPPKPKDTLMISRTPEVTCVVVDV |
| signal export | SHEDPEVKFNWYVDGVEVHNAKTKPREEQYNSTYRVVSVLTVLHQDW |
| sequence or tag): | LNGKEYKCKVSNKALGAPIEKTISKAKGQPREPQVYTLPPCRDELTKNQ |
| SEQ ID NO: 84 | VSLWCLVKGFYPSDIAVEWESNGQPENNYKTTPPVLDSDGSFFLYSKL |
| | TVDKSRWQQGNVFSCSVMHEALHNHYTQKSLSLSPGGHHHHHH* |
| Teplizumab LC: | MGWSCIILFLVATATGVHSDIQMTQSPSSLSASVGDRVTITCSASSSVS |
| SEQ ID NO: 85 | YMNWYQQTPGKAPKRWIYDTSKLASGVPSRFSGSGSGTDYTFTISSLQ |
| Teplizumab LC | PEDIATYYCQQWSSNPFTFGQGTKLQITRTVAAPSVFIFPPSDEQLKSG |
| (no signal export | TASVVCLLNNFYPREAKVQWKVDNALQSGNSQESVTEQDSKDSTYSL |
| sequence): | SSTLTLSKADYEKHKVYACEVTHQGLSSPVTKSFNRGEC* |
| SEQ ID NO: 86 | |
| Durvalumab HC | MGWSCIILFLVATATGVHSEVQLVESGGGLVQPGGSLRLSCAASGFTF |
| knobs 6xHis: | SRYWMSWVRQAPGKGLEWVANIKQDGSEKYYVDSVKGRFTISRDNAK |
| SEQ ID NO: 87 | NSLYLQMNSLRAEDTAVYYCAREGGWFGELAFDYWGQGTLVTVSSAS |
| Durvalumab HC | TKGPSVFPLAPSSKSTSGGTAALGCLVKDYFPEPVTVSWNSGALTSGV |
| knobs (no signal | HTFPAVLQSSGLYSLSSVVTVPSSSLGTQTYICNVNHKPSNTKVDKRVE |
| export sequence | PKSCDKTHTCPPCPAPEFEGGPSVFLFPPKPKDTLMISRTPEVTCVVVD |
| or tag): | VSHEDPEVKFNWYVDGVEVHNAKTKPREEQYNSTYRVVSVLTVLHQD |
| SEQ ID NO: 88 | WLNGKEYKCKVSNKALPASIEKTISKAKGQPREPQVYTLPPCREEMTK |
| | NQVSLWCLVKGFYPSDIAVEWESNGQPENNYKTTPPVLDSDGSFFLYS |
| | KLTVDKSRWQQGNVFSCSVMHEALHNHYTQKSLSLSPGGHHHHHH* |
| Durvalumab LC: | MGWSCIILFLVATATGVHSEIVLTQSPGTLSLSPGERATLSCRASQRVS |
| SEQ ID NO: 89 | SSYLAWYQQKPGQAPRLLIYDASSRATGIPDRFSGSGSGTDFTLTISRL |
| Durvalumab LC | EPEDFAVYYCQQYGSLPWTFGQGTKVEIKRTVAAPSVFIFPPSDEQLK |
| (no signal export | SGTASVVCLLNNFYPREAKVQWKVDNALQSGNSQESVTEQDSKDSTY |
| sequence): | SLSSTLTLSKADYEKHKVYACEVTHQGLSSPVTKSFNRGEC* |
| SEQ ID NO: 90 | |
| Galectin-1 LALA | MLLLLLPLLWGRERAEGQACGLVASNLNLKPGECLRVRGEVAPDAKSE |
| Holes Fc HA: | VLNLGKDSNNLCLHFNPRFNAHGDANTIVCNSKDGGAWGTEQREAVE |
| SEQ ID NO: 91 | PFQPGSVAEVCITFDQANLTKLPDGYEFKFPNRLNLEAINYMAADGDFK |
| Galectin-1 LALA | IKCVAFDGGGGGPKSCDKTHTCPPCPAPEAAGGPSVFLFPPKPKDTLM |
| Holes Fc HA (no | ISRTPEVTCVVVDVSHEDPEVKFNWYVDGVEVHNAKTKPREEQYNSTY |
| signal export | RVVSVLTVLHQDWLNGKEYKCKVSNKALGAPIEKTISKAKGQPREPQV |
| sequence or tag): | CTLPPSRDELTKNQVSLSCAVKGFYPSDIAVEWESNGQPENNYKTTPP |
| SEQ ID NO: 92 | VLDSDGSFFLVSKLTVDKSRWQQGNVFSCSVMHEALHNHYTQKSLSL |
| Galectin-1 LALA | SPGGYPYDVPDYA* |
| Holes Fc HA | |
| Binding Domain: | |
| SEQ ID NO: 93 | |
| Galectin-3 LALA | MLLLLLPLLWGRERAEGQADNFSLHDALSGSGNPNPQGWPGAWGNQ |
| Holes Fc HA: | PAGAGGYPGASYPGAYPGQAPPGAYPGQAPPGAYHGAPGAYPGAPA |
| SEQ ID NO: 94 | PGVYPGPPSGPGAYPSSGQPSAPGAYPATGPYGAPAGPLIVPYNLPLP |
| Galectin-3 LALA | GGVVPRMLITILGTVKPNANRIALDFQRGNDVAFHFNPRFNENNRRVIV |
| Holes Fc HA (no | CNTKLDNNWGREERQSVFPFESGKPFKIHVLVEPDHFKVAVNDAHLLQ |
| signal export | YNHRVKKLNEISKLGISGDIDLTSASYTMIGGGGGPKSCDKTHTCPPCP |
| sequence or tag): | APEAAGGPSVFLFPPKPKDTLMISRTPEVTCVVVDVSHEDPEVKFNWY |
| SEQ ID NO: 95 | VDGVEVHNAKTKPREEQYNSTYRVVSVLTVLHQDWLNGKEYKCKVSN |
| Galectin-3 LALA | KALGAPIEKTISKAKGQPREPQVCTLPPSRDELTKNQVSLSCAVKGFYP |
| Holes Fc HA | SDIAVEWESNGQPENNYKTTPPVLDSDGSFFLVSKLTVDKSRWQQGN |
| Binding Domain: | VFSCSVMHEALHNHYTQKSLSLSPGGYPYDVPDYA* |
| SEQ ID NO: 96 | |

### Nucleotide Sequences

The nucleotide sequences encoding the AbLecs and antibodies employed herein are show in the table below. Sequences include export signal peptides, peptide tags such as the hexahistidine tag and HA tag, and stop codons.

| | | SEQ |
|---|---|---|
| | | ID |
| Protein name | Nucleotide sequence | NO |
| Trastuzumab- | ATGGGTTGGAGCCTCATCTTGCTCTTCCTTGTCGCTGTTGCTA | 97 |
| KNOB heavy | CGCGTGTCCACTCCGAAGTGCAGCTGGTGGAGTCTGGCGGA | |
| chain | GGACTGGTGCAGCCAGGGGGCAGCCTGAGACTGTCTTGCGC | |
| | CGCCTCCGGCTTCAACATCAAGGACACCTACATCCACTGGGT | |
| | CCGCCAGGCACCAGGCAAGGGACTGGAATGGGTGGCCCGG | |
| | ATCTACCCTACCAACGGCTACACCAGATACGCCGACTCCGTG | |
| | AAGGGCCGGTTCACCATCTCCGCCGACACCTCCAAGAACACC | |
| | GCCTACCTGCAGATGAATTCCCTGAGGGCCGAGGACACCGC | |
| | CGTGTACTACTGCTCCAGATGGGGAGGCGACGGCTTCTACG | |
| | CCATGGACTACTGGGGCCAGGGCACCCTGGTCACAGTGTCC | |
| | TCTGCTAGCACCAAGGGCCCATCGGTCTTCCCCCTGGCACCC | |
| | TCCTCCAAGAGCACCTCTGGGGGCACAGCGGCCCTGGGCTG | |
| | CCTGGTCAAGGACTACTTCCCCGAACCGGTGACGGTGTCGTG | |
| | GAACTCAGGCGCCCTGACCAGCGGCGTGCACACCTTCCCGG | |
| | CTGTCCTACAGTCCTCAGGACTCTACTCCCTCAGCAGCGTGG | |
| | TGACCGTGCCCTCCAGCAGCTTGGGCACCCAGACCTACATCT | |
| | GCAACGTGAATCACAAGCCCAGCAACACCAAGGTGGACAAGA | |
| | AAGTTGAGCCCAAATCTTGTGACAAAACTCACACATGCCCACC | |
| | GTGCCCAGCACCTGAACTCCTGGGGGGACCGTCAGTCTTCCT | |
| | CTTCCCCCCAAAACCCAAGGACACCCTCATGATCTCCCGGAC | |
| | CCCTGAGGTCACATGCGTGGTGGTGGACGTGAGCCACGAAG | |
| | ACCCTGAGGTCAAGTTCAACTGGTACGTGGACGGCGTGGAG | |
| | GTGCATAATGCCAAGACAAAGCCGCGGGAGGAGCAGTACAA | |
| | CAGCACGTACCGTGTGGTCAGCGTCCTCACCGTCCTGCACCA | |
| | GGACTGGCTGAATGGCAAGGAGTACAAGTGCAAGGTCTCCAA | |
| | CAAAGCCCTCCCAGCCCCCATCGAGAAAACCATCTCCAAAGC | |
| | CAAAGGGCAGCCCCGAGAACCACAGGTGTACACCCTGCCCC | |
| | CATGCCGGGATGAGCTGACCAAGAACCAGGTCAGCCTGTGG | |
| | TGCCTGGTCAAAGGCTTCTATCCCAGCGACATCGCCGTGGAG | |
| | TGGGAGAGCAATGGGCAGCCGGAGAACAACTACAAGACCAC | |
| | GCCTCCCGTGCTGGACTCCGACGGCTCCTTCTTCCTCTACAG | |
| | CAAGCTCACCGTGGACAAGAGCAGGTGGCAGCAGGGGAACG | |
| | TCTTCTCATGCTCCGTGATGCATGAGGCTCTGCACAACCACTA | |
| | CACGCAGAAGAGCCTCTCCCTGTCTCCGGGTGGGCATCACC | |
| | ACCATCACCATTGA | |
| Trastuzumab light | ATGAGGGTCCCCGCTCAGCTCCTGGGGCTCCTGCTGCTCTG | 98 |
| chain | GCTCCCAGGTGCACGATGTGACATCCAGATGACCCAGTCCCC | |
| | CTCCTCCCTGTCTGCCTCCGTGGGCGACAGAGTGACCATCAC | |
| | CTGTCGGGCCTCCCAGGATGTGAACACCGCCGTGGCCTGGT | |
| | ATCAGCAGAAGCCTGGCAAGGCCCCTAAGCTGCTGATCTACT | |
| | CCGCCTCCTTCCTGTACTCCGGCGTGCCCTCCCGGTTCTCCG | |
| | GCTCCAGATCCGGCACCGACTTCACCCTGACCATCTCCAGCC | |
| | TGCAGCCTGAGGACTTCGCCACCTACTACTGCCAGCAGCACT | |
| | ACACCACCCCTCCAACCTTCGGCCAGGGCACCAAGGTGGAG | |
| | ATCAAGCGTACGGTGGCTGCACCATCTGTCTTCATCTTCCCG | |
| | CCATCTGATGAGCAGTTGAAATCTGGAACTGCCTCTGTTGTGT | |
| | GCCTGCTGAATAACTTCTATCCCAGAGAGGCCAAAGTACAGT | |
| | GGAAGGTGGATAACGCCCTCCAATCGGGTAACTCCCAGGAG | |
| | AGTGTCACAGAGCAGGACAGCAAGGACAGCACCTACAGCCT | |
| | CAGCAGCACCCTGACGCTGAGCAAAGCAGACTACGAGAAACA | |
| | CAAAGTCTACGCCTGCGAAGTCACCCATCAGGGCCTGAGCTC | |
| | GCCCGTCACAAAGAGCTTCAACAGGGGAGAGTGTTGA | |
| Rituximab-KNOB | ATGGGATGGTCATGTATCATCCTTTTTCTAGTAGCAACTGCAA | 99 |
| heavy chain | CCGGTGTACATTCCCAGGTACAACTGCAGCAGCCTGGGGCT | |
| | GAGCTGGTGAAGCCTGGGGCCTCAGTGAAGATGTCCTGCAA | |
| | GGCTTCTGGCTACACATTTACCAGTTACAATATGCACTGGGTA | |
| | AAACAGACACCTGGTCGGGGCCTGGAATGGATTGGAGCTATT | |
| | TATCCCGGAAATGGTGATACTTCCTACAATCAGAAGTTCAAAG | |
| | GCAAGGCCACATTGACTGCAGACAAATCCTCCAGCACAGCCT | |
| | ACATGCAGCTCAGCAGCCTGACATCTGAGGACTCTGCGGTCT | |
| | ATTACTGTGCAAGATCGACTTACTACGGCGGTGACTGGTACTT | |
| | CAATGTCTGGGGCGCAGGGACCACGGTCACCGTCTCTGCAG | |
| | CTAGCACCAAGGGCCCATCGGTCTTCCCCCTGGCACCCTCCT | |
| | CCAAGAGCACCTCTGGGGGCACAGCGGCCCTGGGCTGCCTG | |
| | GTCAAGGACTACTTCCCCGAACCGGTGACGGTGTCGTGGAAC | |
| | TCAGGCGCCCTGACCAGCGGCGTGCACACCTTCCCGGCTGT | |
| | CCTACAGTCCTCAGGACTCTACTCCCTCAGCAGCGTGGTGAC | |
| | CGTGCCCTCCAGCAGCTTGGGCACCCAGACCTACATCTGCAA | |
| | CGTGAATCACAAGCCCAGCAACACCAAGGTGGACAAGAAAGC | |
| | AGAGCCCAAATCTTGTGACAAAACTCACACATGCCCACCGTG | |
| | CCCAGCACCTGAACTCCTGGGGGGACCGTCAGTCTTCCTCTT | |
| | CCCCCCAAAACCCAAGGACACCCTCATGATCTCCCGGACCCC | |
| | TGAGGTCACATGCGTGGTGGTGGACGTGAGCCACGAAGACC | |
| | CTGAGGTCAAGTTCAACTGGTACGTGGACGGCGTGGAGGTG | |
| | CATAATGCCAAGACAAAGCCGCGGGAGGAGCAGTACAACAG | |
| | CACGTACCGTGTGGTCAGCGTCCTCACCGTCCTGCACCAGGA | |
| | CTGGCTGAATGGCAAGGAGTACAAGTGCAAGGTCTCCAACAA | |
| | AGCCCTCCCAGCCCCCATCGAGAAAACCATCTCCAAAGCCAA | |
| | AGGGCAGCCCCGAGAACCACAGGTGTACACCCTGCCCCCAT | |
| | GCCGGGATGAGCTGACCAAGAACCAGGTCAGCCTGTGGTGC | |
| | CTGGTCAAAGGCTTCTATCCCAGCGACATCGCCGTGGAGTGG | |
| | GAGAGCAATGGGCAGCCGGAGAACAACTACAAGACCACGCC | |
| | TCCCGTGCTGGACTCCGACGGCTCCTTCTTCCTCTACAGCAA | |
| | GCTCACCGTGGACAAGAGCAGGTGGCAGCAGGGGAACGTCT | |
| | TCTCATGCTCCGTGATGCATGAGGCTCTGCACAACCACTACA | |
| | CGCAGAAGAGCCTCTCCCTGTCTCCGGGTGGGCATCACCAC | |
| | CATCACCATTGA | |
| Rituximab light | ATGGGATGGTCATGTATCATCCTTTTTCTAGTAGCAACTGCAA | 100 |
| chain | CCGGTGTACATTCACAAATTGTTCTCTCCCAGTCTCCAGCAAT | |
| | CCTGTCTGCATCTCCAGGGGAGAAGGTCACAATGACTTGCAG | |
| | GGCCAGCTCAAGTGTAAGTTACATCCACTGGTTCCAGCAGAA | |
| | GCCAGGATCCTCCCCCAAACCCTGGATTTATGCCACATCCAA | |
| | CCTGGCTTCTGGAGTCCCTGTTCGCTTCAGTGGCAGTGGGTC | |
| | TGGGACTTCTTACTCTCTCACAATCAGCAGAGTGGAGGCTGA | |
| | AGATGCTGCCACTTATTACTGCCAGCAGTGGACTAGTAACCC | |
| | ACCCACGTTCGGAGGGGGGACCAAGCTGGAAATCAAACGTA | |
| | CGGTGGCTGCACCATCTGTCTTCATCTTCCCGCCATCTGATG | |
| | AGCAGTTGAAATCTGGAACTGCCTCTGTTGTGTGCCTGCTGA | |
| | ATAACTTCTACCCCAGAGAAGCCAAAGTGCAGTGGAAGGTGG | |
| | ACAACGCCCTGCAGAGCGGAAACAGCCAGGAAAGCGTGACA | |
| | GAGCAGGATTCCAAGGATTCCACATACAGCCTGAGCAGCACA | |
| | CTGACACTGTCCAAGGCCGACTACGAGAAGCACAAGGTGTAC | |
| | GCCTGCGAAGTGACACACCAGGGACTGTCCTCCCCTGTGACA | |
| | AAGAGCTTCAACAGAGGAGAATGCTGA | |
| Cetuximab KNOB | ATGGGATGGTCATGTATCATCCTTTTTCTAGTAGCAACTGCAA | 101 |
| Heavy chain | CCGGTGTACATTCCCAGGTGCAGCTGAAACAGAGCGGCCCG | |
| | GGCCTGGTGCAGCCGAGCCAGAGCCTGAGCATTACCTGCAC | |
| | CGTGAGCGGCTTTAGCCTGACCAACTATGGCGTGCATTGGGT | |
| | GCGCCAGAGCCCGGGCAAAGGCCTGGAATGGCTGGGCGTG | |
| | ATTTGGAGCGGCGGCAACACCGATTATAACACCCCGTTTACC | |
| | AGCCGCCTGAGCATTAACAAAGATAACAGCAAAAGCCAGGTG | |
| | TTTTTTAAAATGAACAGCCTGCAGAGCAACGATACCGCGATTT | |
| | ATTATTGCGCGCGCGCGCTGACCTATTATGATTATGAATTTGC | |
| | GTATTGGGGCCAGGGCACCCTGGTGACCGTGAGCGCGGCTA | |
| | GCACCAAGGGCCCATCGGTCTTCCCCCTGGCACCCTCCTCCA | |
| | AGAGCACCTCTGGGGGCACAGCGGCCCTGGGCTGCCTGGTC | |
| | AAGGACTACTTCCCCGAACCGGTGACGGTGTCGTGGAACTCA | |
| | GGCGCCCTGACCAGCGGCGTGCACACCTTCCCGGCTGTCCT | |
| | ACAGTCCTCAGGACTCTACTCCCTCAGCAGCGTGGTGACCGT | |
| | GCCCTCCAGCAGCTTGGGCACCCAGACCTACATCTGCAACGT | |
| | GAATCACAAGCCCAGCAACACCAAGGTGGACAAGAAAGTTGA | |
| | GCCCAAATCTTGTGACAAAACTCACACATGCCCACCGTGCCC | |
| | AGCACCTGAACTCCTGGGGGGACCGTCAGTCTTCCTCTTCCC | |
| | CCCAAAACCCAAGGACACCCTCATGATCTCCCGGACCCCTGA | |
| | GGTCACATGCGTGGTGGTGGACGTGAGCCACGAAGACCCTG | |
| | AGGTCAAGTTCAACTGGTACGTGGACGGCGTGGAGGTGCATA | |
| | ATGCCAAGACAAAGCCGCGGGAGGAGCAGTACAACAGCACG | |
| | TACCGTGTGGTCAGCGTCCTCACCGTCCTGCACCAGGACTGG | |
| | CTGAATGGCAAGGAGTACAAGTGCAAGGTCTCCAACAAAGCC | |
| | CTCCCAGCCCCCATCGAGAAAACCATCTCCAAAGCCAAAGGG | |
| | CAGCCCCGAGAACCACAGGTGTACACCCTGCCCCCATGCCG | |
| | GGATGAGCTGACCAAGAACCAGGTCAGCCTGTGGTGCCTGG | |
| | TCAAAGGCTTCTATCCCAGCGACATCGCCGTGGAGTGGGAGA | |
| | GCAATGGGCAGCCGGAGAACAACTACAAGACCACGCCTCCC | |
| | GTGCTGGACTCCGACGGCTCCTTCTTCCTCTACAGCAAGCTC | |
| | ACCGTGGACAAGAGCAGGTGGCAGCAGGGGAACGTCTTCTC | |
| | ATGCTCCGTGATGCATGAGGCTCTGCACAACCACTACACGCA | |
| | GAAGAGCCTCTCCCTGTCTCCGGGTGGGCATCACCACCATCA | |
| | CCATTGA | |
| Cetuximab light | ATGGGATGGTCATGTATCATCCTTTTTCTAGTAGCAACTGCAA | 102 |
| chain | CCGGTGTACATTCCGACATCCTGCTCACCCAGAGCCCGGTGA | |
| | TCCTGTCGGTCAGCCCCGGCGAGCGGGTGAGCTTCAGCTGC | |
| | CGCGCCAGCCAGTCGATCGGGACGAACATCCACTGGTACCA | |
| | GCAGCGGACCAACGGCAGCCCCCGCCTGCTCATCAAGTACG | |
| | CGAGCGAGAGCATCAGCGGGATCCCCTCGCGGTTCAGCGGC | |
| | AGCGGGAGCGGCACCGACTTCACCCTGAGCATCAACAGCGT | |
| | GGAGTCGGAGGACATCGCCGACTACTACTGCCAGCAGAACA | |
| | ACAACTGGCCGACGACCTTCGGCGCCGGGACCAAGCTGGAG | |
| | CTCAAGCGCACCGTCGCCGCGCCCAGCGTGTTCATCTTCCC | |
| | GCCCAGCGACGAGCAGCTGAAGAGCGGCACGGCCAGCGTG | |
| | GTCTGCCTGCTCAACAACTTCTACCCCCGGGAGGCCAAGGTG | |
| | CAGTGGAAGGTGGACAACGCCCTGCAGTCGGGGAACAGCCA | |
| | GGAGAGCGTCACCGAGCAGGACAGCAAGGACAGCACCTACA | |
| | GCCTGTCGAGCACCCTCACGCTGAGCAAGGCCGACTACGAG | |
| | AAGCACAAGGTGTACGCGTGCGAGGTGACCCACCAGGGCCT | |
| | GAGCAGCCCCGTCACCAAGTCGTTCAACCGCGGAGAATGCT | |
| | GA | |
| Siglec-7 HOLES- | ATGCTGCTGCTGCTGCTGCTGCCCCTGCTCTGGGGGAGGGA | 103 |
| Fc | GAGGGTGGAAGGACAGAAGAGTAACCGGAAGGATTACTCGC | |
| | TGACGATGCAGAGTTCCGTGACCGTGCAAGAGGGCATGTGT | |
| | GTCCATGTGCGCTGCTCCTTCTCCTACCCAGTGGACAGCCAG | |
| | ACTGACTCTGACCCAGTTCATGGCTACTGGTTCCGGGCAGGG | |
| | AATGATATAAGCTGGAAGGCTCCAGTGGCCACAAACAACCCA | |
| | GCTTGGGCAGTGCAGGAGGAAACTCGGGACCGATTCCACCT | |
| | CCTTGGGGACCCACAGACCAAAAATTGCACCCTGAGCATCAG | |
| | AGATGCCAGAATGAGTGATGCGGGGAGATACTTCTTTCGTAT | |
| | GGAGAAAGGAAATATAAAATGGAATTATAAATATGACCAGCTC | |
| | TCTGTGAACGTGACAGCCTTGACCCACAGGCCCAACATCCTT | |
| | ATCCCCGGTACCCTGGAGTCTGGCTGCTTCCAGAATCTGACC | |
| | TGCTCTGTGCCCTGGGCCTGTGAGCAGGGGACGCCCCCTAT | |
| | GATCTCCTGGATGGGGACCTCTGTGTCCCCCCTGCACCCCTC | |
| | CACCACCCGCTCCTCAGTGCTCACCCTCATCCCACAGCCCCA | |
| | GCACCACGGCACCAGCCTCACCTGTCAGGTGACCTTGCCTG | |
| | GGGCCGGCGTGACCACGAACAGGACCATCCAACTCAATGTG | |
| | TCCTACCCTCCTCAGAACTTGACTGTGACTGTCTTCCAAGGAG | |
| | AAGGCACAGCATCCACAGCTCTGGGGAACAGCTCATCTCTTT | |
| | CAGTCCTAGAGGGCCAGTCTCTGCGCTTGGTCTGTGCTGTTG | |
| | ACAGCAATCCCCCTGCCAGGCTGAGCTGGACCTGGAGGAGT | |
| | CTGACCCTGTACCCCTCACAGCCCTCAAACCCTCTGGTACTG | |
| | GAGCTGCAAGTGCACCTGGGGGATGAAGGGGAATTCACCTG | |
| | TCGAGCTCAGAACTCTCTGGGTTCCCAGCACGTTTCCCTGAA | |
| | CCTCTCCCTGCAACAGGAGTACACAGGCAAAATGAGGCCTGT | |
| | ATCAGGAGGCGGAGGTGGAGGCCCCAAATCTTGTGACAAAA | |
| | CTCACACATGCCCACCGTGCCCAGCACCTGAACTCCTGGGG | |
| | GGACCGTCAGTCTTCCTCTTCCCCCCAAAACCCAAGGACACC | |
| | CTCATGATCTCCCGGACCCCTGAGGTCACATGCGTGGTGGTG | |
| | GACGTGAGCCACGAAGACCCTGAGGTCAAGTTCAACTGGTAC | |
| | GTGGACGGCGTGGAGGTGCATAATGCCAAGACAAAGCCGCG | |
| | GGAGGAGCAGTACAACAGCACGTACCGTGTGGTCAGCGTCC | |
| | TCACCGTCCTGCACCAGGACTGGCTGAATGGCAAGGAGTACA | |
| | AGTGCAAGGTCTCCAACAAAGCCCTCCCAGCCCCCATCGAGA | |
| | AAACCATCTCCAAAGCCAAAGGGCAGCCCCGAGAACCACAG | |
| | GTGTGCACCCTGCCCCCATCCCGGGATGAGCTGACCAAGAA | |
| | CCAGGTCAGCCTGAGCTGCGCGGTCAAAGGCTTCTATCCCAG | |
| | CGACATCGCCGTGGAGTGGGAGAGCAATGGGCAGCCGGAGA | |
| | ACAACTACAAGACCACGCCTCCCGTGCTGGACTCCGACGGCT | |
| | CCTTCTTCCTCGTGAGCAAGCTCACCGTGGACAAGAGCAGGT | |
| | GGCAGCAGGGGAACGTCTTCTCATGCTCCGTGATGCATGAG | |
| | GCTCTGCACAACCACTACACGCAGAAGAGCCTCTCCCTGTCT | |
| | CCGGGTGGGTACCCATACGATGTTCCAGATTACGCTTGA | |
| Siglec-7 R→A | ATGCTGCTGCTGCTGCTGCTGCCCCTGCTCTGGGGGAGGGA | 104 |
| HOLES-Fc | GAGGGTGGAAGGACAGAAGAGTAACCGGAAGGATTACTCGC | |
| | TGACGATGCAGAGTTCCGTGACCGTGCAAGAGGGCATGTGT | |
| | GTCCATGTGCGCTGCTCCTTCTCCTACCCAGTGGACAGCCAG | |
| | ACTGACTCTGACCCAGTTCATGGCTACTGGTTCCGGGCAGGG | |
| | AATGATATAAGCTGGAAGGCTCCAGTGGCCACAAACAACCCA | |
| | GCTTGGGCAGTGCAGGAGGAAACTCGGGACCGATTCCACCT | |
| | CCTTGGGGACCCACAGACCAAAAATTGCACCCTGAGCATCAG | |
| | AGATGCCAGAATGAGTGATGCGGGGAGATACTTCTTTGCCAT | |
| | GGAGAAAGGAAATATAAAATGGAATTATAAATATGACCAGCTC | |
| | TCTGTGAACGTGACAGCCTTGACCCACAGGCCCAACATCCTT | |
| | ATCCCCGGTACCCTGGAGTCTGGCTGCTTCCAGAATCTGACC | |
| | TGCTCTGTGCCCTGGGCCTGTGAGCAGGGGACGCCCCCTAT | |
| | GATCTCCTGGATGGGGACCTCTGTGTCCCCCCTGCACCCCTC | |
| | CACCACCCGCTCCTCAGTGCTCACCCTCATCCCACAGCCCCA | |
| | GCACCACGGCACCAGCCTCACCTGTCAGGTGACCTTGCCTG | |
| | GGGCCGGCGTGACCACGAACAGGACCATCCAACTCAATGTG | |
| | TCCTACCCTCCTCAGAACTTGACTGTGACTGTCTTCCAAGGAG | |
| | AAGGCACAGCATCCACAGCTCTGGGGAACAGCTCATCTCTTT | |
| | CAGTCCTAGAGGGCCAGTCTCTGCGCTTGGTCTGTGCTGTTG | |
| | ACAGCAATCCCCCTGCCAGGCTGAGCTGGACCTGGAGGAGT | |
| | CTGACCCTGTACCCCTCACAGCCCTCAAACCCTCTGGTACTG | |
| | GAGCTGCAAGTGCACCTGGGGGATGAAGGGGAATTCACCTG | |
| | TCGAGCTCAGAACTCTCTGGGTTCCCAGCACGTTTCCCTGAA | |
| | CCTCTCCCTGCAACAGGAGTACACAGGCAAAATGAGGCCTGT | |
| | ATCAGGAGGCGGAGGTGGAGGCCCCAAATCTTGTGACAAAA | |
| | CTCACACATGCCCACCGTGCCCAGCACCTGAACTCCTGGGG | |
| | GGACCGTCAGTCTTCCTCTTCCCCCCAAAACCCAAGGACACC | |
| | CTCATGATCTCCCGGACCCCTGAGGTCACATGCGTGGTGGTG | |
| | GACGTGAGCCACGAAGACCCTGAGGTCAAGTTCAACTGGTAC | |
| | GTGGACGGCGTGGAGGTGCATAATGCCAAGACAAAGCCGCG | |
| | GGAGGAGCAGTACAACAGCACGTACCGTGTGGTCAGCGTCC | |
| | TCACCGTCCTGCACCAGGACTGGCTGAATGGCAAGGAGTACA | |
| | AGTGCAAGGTCTCCAACAAAGCCCTCCCAGCCCCCATCGAGA | |
| | AAACCATCTCCAAAGCCAAAGGGCAGCCCCGAGAACCACAG | |
| | GTGTGCACCCTGCCCCCATCCCGGGATGAGCTGACCAAGAA | |
| | CCAGGTCAGCCTGAGCTGCGCGGTCAAAGGCTTCTATCCCAG | |
| | CGACATCGCCGTGGAGTGGGAGAGCAATGGGCAGCCGGAGA | |
| | ACAACTACAAGACCACGCCTCCCGTGCTGGACTCCGACGGCT | |
| | CCTTCTTCCTCGTGAGCAAGCTCACCGTGGACAAGAGCAGGT | |
| | GGCAGCAGGGGAACGTCTTCTCATGCTCCGTGATGCATGAG | |
| | GCTCTGCACAACCACTACACGCAGAAGAGCCTCTCCCTGTCT | |
| | CCGGGTGGGTACCCATACGATGTTCCAGATTACGCTTGA | |
| Siglec-9 HOLES- | ATGCTGCTGCTGCTGCTGCCCCTGCTCTGGGGGAGGGAGAG | 105 |
| Fc | GGCGGAAGGACAGACAAGTAAACTGCTGACGATGCAGAGTTC | |
| | CGTGACGGTGCAGGAAGGCCTGTGTGTCCATGTGCCCTGCT | |
| | CCTTCTCCTACCCCTCGCATGGCTGGATTTACCCTGGCCCAG | |
| | TAGTTCATGGCTACTGGTTCCGGGAAGGGGCCAATACAGACC | |
| | AGGATGCTCCAGTGGCCACAAACAACCCAGCTCGGGCAGTG | |
| | TGGGAGGAGACTCGGGACCGATTCCACCTCCTTGGGGACCC | |
| | ACATACCAAGAATTGCACCCTGAGCATCAGAGATGCCAGAAG | |
| | AAGTGATGCGGGGAGATACTTCTTTCGTATGGAGAAAGGAAG | |
| | TATAAAATGGAATTATAAACATCACCGGCTCTCTGTGAATGTG | |
| | ACAGCCTTGACCCACAGGCCCAACATCCTCATCCCAGGCACC | |
| | CTGGAGTCCGGCTGCCCCCAGAATCTGACCTGCTCTGTGCCC | |
| | TGGGCCTGTGAGCAGGGGACACCCCCTATGATCTCCTGGATA | |
| | GGGACCTCCGTGTCCCCCCTGGACCCCTCCACCACCCGCTC | |
| | CTCGGTGCTCACCCTCATCCCACAGCCCCAGGACCATGGCAC | |
| | CAGCCTCACCTGTCAGGTGACCTTCCCTGGGGCCAGCGTGA | |
| | CCACGAACAAGACCGTCCATCTCAACGTGTCCTACCCGCCTC | |
| | AGAACTTGACCATGACTGTCTTCCAAGGAGACGGCACAGTAT | |
| | CCACAGTCTTGGGAAATGGCTCATCTCTGTCACTCCCAGAGG | |
| | GCCAGTCTCTGCGCCTGGTCTGTGCAGTTGATGCAGTTGACA | |
| | GCAATCCCCCTGCCAGGCTGAGCCTGAGCTGGAGAGGCCTG | |
| | ACCCTGTGCCCCTCACAGCCCTCAAACCCGGGGGTGCTGGA | |
| | GCTGCCTTGGGTGCACCTGAGGGATGCAGCTGAATTCACCTG | |
| | CAGAGCTCAGAACCCTCTCGGCTCTCAGCAGGTCTACCTGAA | |
| | CGTCTCCCTGCAGAGCAAAGCCACATCAGGCGGAGGTGGAG | |
| | GCCCCAAATCTTGTGACAAAACTCACACATGCCCACCGTGCC | |
| | CAGCACCTGAACTCCTGGGGGGACCGTCAGTCTTCCTCTTCC | |
| | CCCCAAAACCCAAGGACACCCTCATGATCTCCCGGACCCCTG | |
| | AGGTCACATGCGTGGTGGTGGACGTGAGCCACGAAGACCCT | |
| | GAGGTCAAGTTCAACTGGTACGTGGACGGCGTGGAGGTGCA | |
| | TAATGCCAAGACAAAGCCGCGGGAGGAGCAGTACAACAGCA | |
| | CGTACCGTGTGGTCAGCGTCCTCACCGTCCTGCACCAGGACT | |
| | GGCTGAATGGCAAGGAGTACAAGTGCAAGGTCTCCAACAAAG | |
| | CCCTCCCAGCCCCCATCGAGAAAACCATCTCCAAAGCCAAAG | |
| | GGCAGCCCCGAGAACCACAGGTGTGCACCCTGCCCCCATCC | |
| | CGGGATGAGCTGACCAAGAACCAGGTCAGCCTGAGCTGCGC | |
| | GGTCAAAGGCTTCTATCCCAGCGACATCGCCGTGGAGTGGG | |
| | AGAGCAATGGGCAGCCGGAGAACAACTACAAGACCACGCCT | |
| | CCCGTGCTGGACTCCGACGGCTCCTTCTTCCTCGTGAGCAAG | |
| | CTCACCGTGGACAAGAGCAGGTGGCAGCAGGGGAACGTCTT | |
| | CTCATGCTCCGTGATGCATGAGGCTCTGCACAACCACTACAC | |
| | GCAGAAGAGCCTCTCCCTGTCTCCGGGTGGGTACCCATACGA | |
| | TGTTCCAGATTACGCTTGA | |
| Siglec-9 | ATGCTGCTGCTGCTGCTGCCCCTGCTCTGGGGGAGGGAGAG | 106 |
| R120→A | GGCGGAAGGACAGACAAGTAAACTGCTGACGATGCAGAGTTC | |
| HOLES-Fc | CGTGACGGTGCAGGAAGGCCTGTGTGTCCATGTGCCCTGCT | |
| | CCTTCTCCTACCCCTCGCATGGCTGGATTTACCCTGGCCCAG | |
| | TAGTTCATGGCTACTGGTTCCGGGAAGGGGCCAATACAGACC | |
| | AGGATGCTCCAGTGGCCACAAACAACCCAGCTCGGGCAGTG | |
| | TGGGAGGAGACTCGGGACCGATTCCACCTCCTTGGGGACCC | |
| | ACATACCAAGAATTGCACCCTGAGCATCAGAGATGCCAGAAG | |
| | AAGTGATGGGGGGAGATACTTCTTTGCCATGGAGAAAGGAAG | |
| | TATAAAATGGAATTATAAACATCACCGGCTCTCTGTGAATGTG | |
| | ACAGCCTTGACCCACAGGCCCAACATCCTCATCCCAGGCACC | |
| | CTGGAGTCCGGCTGCCCCCAGAATCTGACCTGCTCTGTGCCC | |
| | TGGGCCTGTGAGCAGGGGACACCCCCTATGATCTCCTGGATA | |
| | GGGACCTCCGTGTCCCCCCTGGACCCCTCCACCACCCGCTC | |
| | CTCGGTGCTCACCCTCATCCCACAGCCCCAGGACCATGGCAC | |
| | CAGCCTCACCTGTCAGGTGACCTTCCCTGGGGCCAGCGTGA | |
| | CCACGAACAAGACCGTCCATCTCAACGTGTCCTACCCGCCTC | |
| | AGAACTTGACCATGACTGTCTTCCAAGGAGACGGCACAGTAT | |
| | CCACAGTCTTGGGAAATGGCTCATCTCTGTCACTCCCAGAGG | |
| | GCCAGTCTCTGCGCCTGGTCTGTGCAGTTGATGCAGTTGACA | |
| | GCAATCCCCCTGCCAGGCTGAGCCTGAGCTGGAGAGGCCTG | |
| | ACCCTGTGCCCCTCACAGCCCTCAAACCCGGGGGTGCTGGA | |
| | GCTGCCTTGGGTGCACCTGAGGGATGCAGCTGAATTCACCTG | |
| | CAGAGCTCAGAACCCTCTCGGCTCTCAGCAGGTCTACCTGAA | |
| | CGTCTCCCTGCAGAGCAAAGCCACATCAGGCGGAGGTGGAG | |
| | GCCCCAAATCTTGTGACAAAACTCACACATGCCCACCGTGCC | |
| | CAGCACCTGAACTCCTGGGGGGACCGTCAGTCTTCCTCTTCC | |
| | CCCCAAAACCCAAGGACACCCTCATGATCTCCCGGACCCCTG | |
| | AGGTCACATGCGTGGTGGTGGACGTGAGCCACGAAGACCCT | |
| | GAGGTCAAGTTCAACTGGTACGTGGACGGCGTGGAGGTGCA | |
| | TAATGCCAAGACAAAGCCGCGGGAGGAGCAGTACAACAGCA | |
| | CGTACCGTGTGGTCAGCGTCCTCACCGTCCTGCACCAGGACT | |
| | GGCTGAATGGCAAGGAGTACAAGTGCAAGGTCTCCAACAAAG | |
| | CCCTCCCAGCCCCCATCGAGAAAACCATCTCCAAAGCCAAAG | |
| | GGCAGCCCCGAGAACCACAGGTGTGCACCCTGCCCCCATCC | |
| | CGGGATGAGCTGACCAAGAACCAGGTCAGCCTGAGCTGCGC | |
| | GGTCAAAGGCTTCTATCCCAGCGACATCGCCGTGGAGTGGG | |
| | AGAGCAATGGGCAGCCGGAGAACAACTACAAGACCACGCCT | |
| | CCCGTGCTGGACTCCGACGGCTCCTTCTTCCTCGTGAGCAAG | |
| | CTCACCGTGGACAAGAGCAGGTGGCAGCAGGGGAACGTCTT | |
| | CTCATGCTCCGTGATGCATGAGGCTCTGCACAACCACTACAC | |
| | GCAGAAGAGCCTCTCCCTGTCTCCGGGTGGGTACCCATACGA | |
| | TGTTCCAGATTACGCTTGA | |
| Siglec-10 | ATGCTACTGCCACTGCTGCTGTCCTCGCTGCTGGGGGGGTCC | 107 |
| HOLES-Fc HA | CAGGCTATGGATGGGAGATTCTGGATACGAGTGCAGGAGTCA | |
| | GTGATGGTGCCGGAGGGCCTGTGCATCTCTGTGCCCTGCTCT | |
| | TTCTCCTACCCCCGACAGGACTGGACAGGGTCTACCCCAGCT | |
| | TATGGCTACTGGTTCAAAGCAGTGACTGAGACAACCAAGGGT | |
| | GCTCCTGTGGCCACAAACCACCAGAGTCGAGAGGTGGAAAT | |
| | GAGCACCCGGGGCCGATTCCAGCTCACTGGGGATCCCGCCA | |
| | AGGGGAACTGCTCCTTGGTGATCAGAGACGCGCAGATGCAG | |
| | GATGAGTCACAGTACTTCTTTCGGGTGGAGAGAGGAAGCTAT | |
| | GTGAGATATAATTTCATGAACGATGGGTTCTTTCTAAAAGTAA | |
| | CAGCCCTGACTCAGAAGCCTGATGTCTACATCCCCGAGACCC | |
| | TGGAGCCCGGGCAGCCGGTGACGGTCATCTGTGTGTTTAACT | |
| | GGGCCTTTGAGGAATGTCCACCCCCTTCTTTCTCCTGGACGG | |
| | GGGCTGCCCTCTCCTCCCAAGGAACCAAACCAACGACCTCCC | |
| | ACTTCTCAGTGCTCAGCTTCACGCCCAGACCCCAGGACCACA | |
| | ACACCGACCTCACCTGCCATGTGGACTTCTCCAGAAAGGGTG | |
| | TGAGCGCACAGAGGACCGTCCGACTCCGTGTGGCCTATGCC | |
| | CCCAGAGACCTTGTTATCAGCATTTCACGTGACAACACGCCA | |
| | GCCCTGGAGCCCCAGCCCCAGGGAAATGTCCCATACCTGGA | |
| | AGCCCAAAAAGGCCAGTTCCTGCGGCTCCTCTGTGCTGCTGA | |
| | CAGCCAGCCCCCTGCCACACTGAGCTGGGTCCTGCAGAACA | |
| | GAGTCCTCTCCTCGTCCCATCCCTGGGGCCCTAGACCCCTGG | |
| | GGCTGGAGCTGCCCGGGGTGAAGGCTGGGGATTCAGGGCG | |
| | CTACACCTGCCGAGCGGAGAACAGGCTTGGCTCCCAGCAGC | |
| | GAGCCCTGGACCTCTCTGTGCAGTATCCTCCAGAGAACCTGA | |
| | GAGTGATGGTTTCCCAAGCAAACAGGACAGTCCTGGAAAACC | |
| | TTGGGAACGGCACGTCTCTCCCAGTACTGGAGGGCCAAAGC | |
| | CTGTGCCTGGTCTGTGTCACACACAGCAGCCCCCCAGCCAG | |
| | GCTGAGCTGGACCCAGAGGGGACAGGTTCTGAGCCCCTCCC | |
| | AGCCCTCAGACCCCGGGGTCCTGGAGCTGCCTCGGGTTCAA | |
| | GTGGAGCACGAAGGAGAGTTCACCTGCCACGCTCGGCACCC | |
| | ACTGGGCTCCCAGCACGTCTCTCTCAGCCTCTCCGTGCACTA | |
| | CTCCCCGAAGCTGCTGGGCCCCTCCTGCTCCTGGGAGGCTG | |
| | AGGGTCTGCACTGCAGCTGCTCCTCCCAGGCCAGCCCGGCC | |
| | CCCTCTCTGCGCTGGTGGCTTGGGGAGGAGCTGCTGGAGGG | |
| | GAACAGCAGCCAGGACTCCTTCGAGGTCACCCCCAGCTCAG | |
| | CCGGGCCCTGGGCCAACAGCTCCCTGAGCCTCCATGGAGGG | |
| | CTCAGCTCCGGCCTCAGGCTCCGCTGTGAGGCCTGGAACGT | |
| | CCATGGGGCCCAGAGTGGATCCATCCTGCAGCTGCCAGATAA | |
| | GAAGGGACTCATCTCAACGGGGGGAGGTGGAGGCCCCAAAT | |
| | CTTGTGACAAAACTCACACATGCCCACCGTGCCCAGCACCTG | |
| | AACTCCTGGGGGGACCGTCAGTCTTCCTCTTCCCCCCAAAAC | |
| | CCAAGGACACCCTCATGATCTCCCGGACCCCTGAGGTCACAT | |
| | GCGTGGTGGTGGACGTGAGCCACGAAGACCCTGAGGTCAAG | |
| | TTCAACTGGTACGTGGACGGCGTGGAGGTGCATAATGCCAAG | |
| | ACAAAGCCGCGGGAGGAGCAGTACAACAGCACGTACCGTGT | |
| | GGTCAGCGTCCTCACCGTCCTGCACCAGGACTGGCTGAATG | |
| | GCAAGGAGTACAAGTGCAAGGTCTCCAACAAAGCCCTCCCAG | |
| | CCCCCATCGAGAAAACCATCTCCAAAGCCAAAGGGCAGCCCC | |
| | GAGAACCACAGGTGTGCACCCTGCCCCCATCCCGGGATGAG | |
| | CTGACCAAGAACCAGGTCAGCCTGAGCTGCGCGGTCAAAGG | |
| | CTTCTATCCCAGCGACATCGCCGTGGAGTGGGAGAGCAATG | |
| | GGCAGCCGGAGAACAACTACAAGACCACGCCTCCCGTGCTG | |
| | GACTCCGACGGCTCCTTCTTCCTCGTGAGCAAGCTCACCGTG | |
| | GACAAGAGCAGGTGGCAGCAGGGGAACGTCTTCTCATGCTC | |
| | CGTGATGCATGAGGCTCTGCACAACCACTACACGCAGAAGAG | |
| | CCTCTCCCTGTCTCCGGGTGGGTACCCATACGATGTTCCAGA | |
| | TTACGCTTGA | |
| Pembro HC | atgggatggtcatgtatcatcctttttctagtagcaactgcaaccggtgtacattcccaagtgc | 108 |
| knobs LALA | aattggtgcagtccggcgttgaagttaaaaagcccggtgcgtccgtgaaggtttcctgtaag | |
| P329G 6xHis | gcgagtggatacaccttcacaaactattatatgtattgggtcaggcaagctccgggacagg | |
| | gtttggaatggatgggaggtatcaatccatccaatggtggtacgaattttaatgagaagttca | |
| | aaaatagagtgactctgactaccgacagttctacgactacagcctacatggaattgaagtc | |
| | cctgcaattcgacgatacggcagtttattattgtgctaggagggattatagattcgacatgggt | |
| | tttgattattggggccaagggactactgtaacagtatcatccgctagcaccaagggcccatc | |
| | ggtcttccccctggcaccctcctccaagagcacctctgggggcacagcggccctgggctgc | |
| | ctggtcaaggactacttccccgaaccggtgacggtgtcgtggaactcaggcgccctgacc | |
| | agcggcgtgcacaccttcccggctgtcctacagtcctcaggactctactccctcagcagcgt | |
| | ggtgaccgtgccctccagcagcttgggcacccagacctacatctgcaacgtgaatcacaa | |
| | gcccagcaacaccaaggtggacaagaaagttgagcccaaatcttgtgacaaaactcaca | |
| | catgcccaccgtgcccagcacctgaagcagccgggggaccgtcagtcttcctcttcccccc | |
| | aaaacccaaggacaccctcatgatctcccggacccctgaggtcacatgcgtggtggtgga | |
| | cgtgagccacgaagaccctgaggtcaagttcaactggtacgtggacggcgtggaggtgc | |
| | ataatgccaagacaaagccgcgggaggagcagtacaacagcacgtaccgtgtggtcag | |
| | cgtcctcaccgtcctgcaccaggactggctgaatggcaaggagtacaagtgcaaggtctc | |
| | caacaaagccctcggagcccccatcgagaaaaccatctccaaagccaaagggcagcc | |
| | ccgagaaccacaggtgtacaccctgcccccatgccgggatgagctgaccaagaaccag | |
| | gtcagcctgtggtgcctggtcaaaggcttctatcccagcgacatcgccgtggagtgggaga | |
| | gcaatgggcagccggagaacaactacaagaccacgcctcccgtgctggactccgacgg | |
| | ctccttcttcctctacagcaagctcaccgtggacaagagcaggtggcagcaggggaacgt | |
| | cttctcatgctccgtgatgcatgaggctctgcacaaccactacacgcagaagagcctctccc | |
| | tgtctccgggtgggcatcaccaccatcaccattga | |
| Pembro LC | atgggatggtcatgtatcatcctttttctagtagcaactgcaaccggtgtacattccgaaattgtt | 109 |
| | ctcactcagtctccggcaacgttgtcactcagtcctggtgagagagctacgttgtcctgccgg | |
| | gctagcaagggagtttcaacatcaggatattcctacctccactggtaccaacagaaaccgg | |
| | gtcaggctccgcgcctcctcatttacctcgcatcctacctggaatctggggtgcccgcgcggt | |
| | tttctgggtctggctctgggactgatttcactctcactatttcctcattggaacccgaggatttt | |
| | gcggtgtactattgccagcactcacgggatctgcctcttacttttggaggaggtactaaagtgga | |
| | aataaaacgcaccgtcgccgcgcccagcgtgttcatcttcccgcccagcgacgagcagct | |
| | gaagagcggcacggccagcgtggtctgcctgctcaacaacttctacccccgggaggcca | |
| | aggtgcagtggaaggtggacaacgccctgcagtcggggaacagccaggagagcgtca | |
| | ccgagcaggacagcaaggacagcacctacagcctgtcgagcaccctcacgctgagcaa | |
| | ggccgactacgagaagcacaaggtgtacgcgtgcgaggtgacccaccagggcctgagc | |
| | agccccgtcaccaagtcgttcaaccgcggagaatgctga | |
| Nivolumab HC | atgggatggtcatgtatcatcctttttctagtagcaactgcaaccggtgtacattcccaagtgc | 110 |
| knobs LALA | agctcgttgaatcaggtgggggtgtggtacaacctgggagatcactgcgactggattgcaa | |
| P329G 6xHis | ggcttccggtatcacgttttcaaactcaggtatgcattgggtccgacaggcaccaggaaaa | |
| | ggactcgagtgggtagccgtaatctggtatgacggctcaaaacgctactatgcggatagtgt | |
| | aaaaggaaggttcacgatctcacgcgataactctaagaacaccctgttccttcagatgaact | |
| | ccctgcgagcagaagatacggcagtttattattgtgcaaccaacgatgattattggggccag | |
| | ggaaccttggttaccgtctcatcagctagcaccaagggcccatcggtcttccccctggcacc | |
| | ctcctccaagagcacctctgggggcacagcggccctgggctgcctggtcaaggactacttc | |
| | cccgaaccggtgacggtgtcgtggaactcaggcgccctgaccagcggcgtgcacaccttc | |
| | ccggctgtcctacagtcctcaggactctactccctcagcagcgtggtgaccgtgccctccag | |
| | cagcttgggcacccagacctacatctgcaacgtgaatcacaagcccagcaacaccaagg | |
| | tggacaagaaagttgagcccaaatcttgtgacaaaactcacacatgcccaccgtgcccag | |
| | cacctgaagcagccgggggaccgtcagtcttcctcttccccccaaaacccaaggacaccc | |
| | tcatgatctcccggacccctgaggtcacatgcgtggtggtggacgtgagccacgaagacc | |
| | ctgaggtcaagttcaactggtacgtggacggcgtggaggtgcataatgccaagacaaagc | |
| | cgcgggaggagcagtacaacagcacgtaccgtgtggtcagcgtcctcaccgtcctgcacc | |
| | aggactggctgaatggcaaggagtacaagtgcaaggtctccaacaaagccctcggagc | |
| | ccccatcgagaaaaccatctccaaagccaaagggcagccccgagaaccacaggtgtac | |
| | accctgcccccatgccgggatgagctgaccaagaaccaggtcagcctgtggtgcctggtc | |
| | aaaggcttctatcccagcgacatcgccgtggagtgggagagcaatgggcagccggaga | |
| | acaactacaagaccacgcctcccgtgctggactccgacggctccttcttcctctacagcaa | |
| | gctcaccgtggacaagagcaggtggcagcaggggaacgtcttctcatgctccgtgatgcat | |
| | gaggctctgcacaaccactacacgcagaagagcctctccctgtctccggggggcatcac | |
| | caccatcaccattga | |
| Nivolumab LC | atgggatggtcatgtatcatcctttttctagtagcaactgcaaccggtgtacattccgagatcgt | 111 |
| | tcttactcaatctcctgcaacgctttccctcagtccaggagaacgcgcgaccctttcatgcag | |
| | agcctctcagagtgtttcctcatatctcgcctggtatcagcaaaaaccagggcaagcacccc | |
| | gcttgttgatttacgacgcgagcaatcgggccacaggcattccagccaggttttcaggaagc | |
| | ggatcaggcaccgatttcacgctgaccattagctcccttgagccagaagatttcgcagtcta | |
| | ctattgtcagcaatcatcaaactggcctcgaaccttcggccagggtacaaaagttgaaata | |
| | aagcgcaccgtcgccgcgcccagcgtgttcatcttcccgcccagcgacgagcagctgaa | |
| | gagcggcacggccagcgtggtctgcctgctcaacaacttctacccccgggaggccaaggt | |
| | gcagtggaaggtggacaacgccctgcagtcggggaacagccaggagagcgtcaccga | |
| | gcaggacagcaaggacagcacctacagcctgtcgagcaccctcacgctgagcaaggcc | |
| | gactacgagaagcacaaggtgtacgcgtgcgaggtgacccaccagggcctgagcagcc | |
| | ccgtcaccaagtcgttcaaccgcggagaatgctga | |
| Siglec-7 Holes | atgctgctgctgctgctgctgcccctgctctgggggagggagagggtggaaggacagaag | 112 |
| LALA P329G Fc | agtaaccggaaggattactcgctgacgatgcagagttccgtgaccgtgcaagagggcatg | |
| HA | tgtgtccatgtgcgctgctccttctcctacccagtggacagccagactgactctgacccagttc | |
| | atggctactggttccgggcagggaatgatataagctggaaggctccagtggccacaaaca | |
| | acccagcttgggcagtgcaggaggaaactcgggaccgattccacctccttggggaccca | |
| | cagaccaaaaattgcaccctgagcatcagagatgccagaatgagtgatgcggggagata | |
| | cttctttcgtatggagaaaggaaatataaaatggaattataaatatgaccagctctctgtgaa | |
| | cgtgacagccttgacccacaggcccaacatccttatccccggtaccctggagtctggctgct | |
| | tccagaatctgacctgctctgtgccctgggcctgtgagcaggggacgccccctatgatctcct | |
| | ggatggggacctctgtgtcccccctgcacccctccaccacccgctcctcagtgctcaccctc | |
| | atcccacagccccagcaccacggcaccagcctcacctgtcaggtgaccttgcctggggcc | |
| | ggcgtgaccacgaacaggaccatccaactcaatgtgtcctaccctcctcagaacttgactgt | |
| | gactgtcttccaaggagaaggcacagcatccacagctctggggaacagctcatctctttca | |
| | gtcctagagggccagtctctgcgcttggtctgtgctgttgacagcaatccccctgccaggctg | |
| | agctggacctggaggagtctgaccctgtacccctcacagccctcaaaccctctggtactgg | |
| | agctgcaagtgcacctgggggatgaaggggaattcacctgtcgagctcagaactctctgg | |
| | gttcccagcacgtttccctgaacctctccctgcaacaggagtacacaggcaaaatgaggcc | |
| | tgtatcaggaggcggaggtggaggccccaaatcttgtgacaaaactcacacatgcccacc | |
| | gtgcccagcacctgaagcagccgggggaccgtcagtcttcctcttccccccaaaacccaa | |
| | ggacaccctcatgatctcccggacccctgaggtcacatgcgtggtggtggacgtgagcca | |
| | cgaagaccctgaggtcaagttcaactggtacgtggacggcgtggaggtgcataatgccaa | |
| | gacaaagccgcgggaggagcagtacaacagcacgtaccgtgtggtcagcgtcctcacc | |
| | gtcctgcaccaggactggctgaatggcaaggagtacaagtgcaaggtctccaacaaagc | |
| | cctcggagcccccatcgagaaaaccatctccaaagccaaagggcagccccgagaacc | |
| | acaggtgtgcaccctgcccccatcccgggatgagctgaccaagaaccaggtcagcctga | |
| | gctgcgcggtcaaaggcttctatcccagcgacatcgccgtggagtgggagagcaatgggc | |
| | agccggagaacaactacaagaccacgcctcccgtgctggactccgacggctccttcttcct | |
| | cgtgagcaagctcaccgtggacaagagcaggtggcagcaggggaacgtcttctcatgctc | |
| | cgtgatgcatgaggctctgcacaaccactacacgcagaagagcctctccctgtctccgggt | |
| | gggtacccatacgatgttccagattacgcttga | |
| Siglec-9 Holes | atgctgctgctgctgctgcccctgctctgggggagggagagggcggaaggacagacaag | 113 |
| LALA P329G Fc | taaactgctgacgatgcagagttccgtgacggtgcaggaaggcctgtgtgtccatgtgccct | |
| HA | gctccttctcctacccctcgcatggctggatttaccctggcccagtagttcatggctactggttc | |
| | cgggaaggggccaatacagaccaggatgctccagtggccacaaacaacccagctcgg | |
| | gcagtgtgggaggagactcgggaccgattccacctccttggggacccacataccaagaat | |
| | tgcaccctgagcatcagagatgccagaagaagtgatgcggggagatacttctttcgtatgg | |
| | agaaaggaagtataaaatggaattataaacatcaccggctctctgtgaatgtgacagcctt | |
| | gacccacaggcccaacatcctcatcccaggcaccctggagtccggctgcccccagaatct | |
| | gacctgctctgtgccctgggcctgtgagcaggggacaccccctatgatctcctggataggg | |
| | acctccgtgtcccccctggacccctccaccacccgctcctcggtgctcaccctcatcccaca | |
| | gccccaggaccatggcaccagcctcacctgtcaggtgaccttccctggggccagcgtgac | |
| | cacgaacaagaccgtccatctcaacgtgtcctacccgcctcagaacttgaccatgactgtct | |
| | tccaaggagacggcacagtatccacagtcttgggaaatggctcatctctgtcactcccaga | |
| | gggccagtctctgcgcctggtctgtgcagttgatgcagttgacagcaatccccctgccaggc | |
| | tgagcctgagctggagaggcctgaccctgtgcccctcacagccctcaaacccgggggtgc | |
| | tggagctgccttgggtgcacctgagggatgcagctgaattcacctgcagagctcagaaccc | |
| | tctcggctctcagcaggtctacctgaacgtctccctgcagagcaaagccacatcaggcgga | |
| | ggtggaggccccaaatcttgtgacaaaactcacacatgcccaccgtgcccagcacctgaa | |
| | gcagccgggggaccgtcagtcttcctcttccccccaaaacccaaggacaccctcatgatct | |
| | cccggacccctgaggtcacatgcgtggtggtggacgtgagccacgaagaccctgaggtc | |
| | aagttcaactggtacgtggacggcgtggaggtgcataatgccaagacaaagccgcggga | |
| | ggagcagtacaacagcacgtaccgtgtggtcagcgtcctcaccgtcctgcaccaggactg | |
| | gctgaatggcaaggagtacaagtgcaaggtctccaacaaagccctcggagcccccatcg | |
| | agaaaaccatctccaaagccaaagggcagccccgagaaccacaggtgtgcaccctgcc | |
| | cccatcccgggatgagctgaccaagaaccaggtcagcctgagctgcgcggtcaaaggct | |
| | tctatcccagcgacatcgccgtggagtgggagagcaatgggcagccggagaacaactac | |
| | aagaccacgcctcccgtgctggactccgacggctccttcttcctcgtgagcaagctcaccgt | |
| | ggacaagagcaggtggcagcaggggaacgtcttctcatgctccgtgatgcatgaggctct | |
| | gcacaaccactacacgcagaagagcctctccctgtctccggggggtacccatacgatgtt | |
| | ccagattacgcttga | |
| Siglec-10Ig2 | atgctactgccactgctgctgtcctcgctgctgggcgggtcccaggctatggatgggagattc | 114 |
| Holes Fc HA | tggatacgagtgcaggagtcagtgatggtgccggagggcctgtgcatctctgtgccctgctc | |
| | tttctcctacccccgacaggactggacagggtctaccccagcttatggctactggttcaaagc | |
| | agtgactgagacaaccaagggtgctcctgtggccacaaaccaccagagtcgagaggtg | |
| | gaaatgagcacccggggccgattccagctcactggggatcccgccaaggggaactgctc | |
| | cttggtgatcagagacgcgcagatgcaggatgagtcacagtacttctttcgggtggagaga | |
| | ggaagctatgtgagatataatttcatgaacgatgggttctttctaaaagtaacagccctgactc | |
| | agaagcctgatgtctacatccccgagaccctggagcccgggcagccggtgacggtcatct | |
| | gtgtgtttaactgggcctttgaggaatgtccacccccttctttctcctggacgggggctgccctc | |
| | tcctcccaaggaaccaaaccaacgacctcccacttctcagtgctcagcttcacgcccagac | |
| | cccaggaccacaacaccgacctcacctgccatgtggacttctccagaaagggtgtgagcg | |
| | cacagaggaccgtccgactccgtgtggcctatgcccccagagaccttgttatcagcatttca | |
| | cgtgacaacacgccagccctggagccccagccccagggaaatgtcccatacctggaag | |
| | cccaaaaaggccagttcctgcggctcctctgtgctgctgacagccagccccctgccacact | |
| | gagctgggtcctgcagaacagagtcctctcctcgtcccatccctggggccctagacccctg | |
| | gggctggagctgcccggggtgaaggctggggattcagggcgctacacctgccgagcgg | |
| | agaacaggcttggctcccagcagcgagccctggacctctctggcggaggtggaggcccc | |
| | aaatcttgtgacaaaactcacacatgcccaccgtgcccagcacctgaactcctgggggga | |
| | ccgtcagtcttcctcttccccccaaaacccaaggacaccctcatgatctcccggacccctga | |
| | ggtcacatgcgtggtggtggacgtgagccacgaagaccctgaggtcaagttcaactggta | |
| | cgtggacggcgtggaggtgcataatgccaagacaaagccgcgggaggagcagtacaa | |
| | cagcacgtaccgtgtggtcagcgtcctcaccgtcctgcaccaggactggctgaatggcaa | |
| | ggagtacaagtgcaaggtctccaacaaagccctcccagcccccatcgagaaaaccatct | |
| | ccaaagccaaagggcagccccgagaaccacaggtgtgcaccctgcccccatcccggg | |
| | atgagctgaccaagaaccaggtcagcctgagctgcgcggtcaaaggcttctatcccagcg | |
| | acatcgccgtggagtgggagagcaatgggcagccggagaacaactacaagaccacgc | |
| | ctcccgtgctggactccgacggctccttcttcctcgtgagcaagctcaccgtggacaagagc | |
| | aggtggcagcaggggaacgtcttctcatgctccgtgatgcatgaggctctgcacaaccact | |
| | acacgcagaagagcctctccctgtctccggggggtacccatacgatgttccagattacgct | |
| | tga | |
| Galectin-9N | atggccttcagcggttcccaggctccctacctgagtccagctgtccccttttctgggactattca | 115 |
| LALAP329G | aggaggtctccaggacggacttcagatcactgtcaatgggaccgttctcagctccagtgga | |
| Holes Fc HA | accaggtttgctgtgaactttcagactggcttcagtggaaatgacattgccttccacttcaacc | |
| | ctcggtttgaagatggagggtacgtggtgtgcaacacgaggcagaacggaagctggggg | |
| | cccgaggagaggaagacacacatgcctttccagaaggggatgccctttgacctctgcttcc | |
| | tggtgcagagctcagatttcaaggtgatggtgaacgggatcctcttcgtgcagtacttccacc | |
| | gcgtgcccttccaccgtgtggacaccatctccgtcaatggctctgtgcagctgtcctacatca | |
| | gcttccagggcggaggtggaggccccaaatcttgtgacaaaactcacacatgcccaccgt | |
| | gcccagcacctgaagcagccgggggaccgtcagtcttcctcttccccccaaaacccaag | |
| | gacaccctcatgatctcccggacccctgaggtcacatgcgtggtggtggacgtgagccac | |
| | gaagaccctgaggtcaagttcaactggtacgtggacggcgtggaggtgcataatgccaag | |
| | acaaagccgcgggaggagcagtacaacagcacgtaccgtgtggtcagcgtcctcaccgt | |
| | cctgcaccaggactggctgaatggcaaggagtacaagtgcaaggtctccaacaaagccc | |
| | tcggagcccccatcgagaaaaccatctccaaagccaaagggcagccccgagaaccac | |
| | aggtgtgcaccctgcccccatcccgggatgagctgaccaagaaccaggtcagcctgagct | |
| | gcgcggtcaaaggcttctatcccagcgacatcgccgtggagtgggagagcaatgggcag | |
| | ccggagaacaactacaagaccacgcctcccgtgctggactccgacggctccttcttcctcg | |
| | tgagcaagctcaccgtggacaagagcaggtggcagcaggggaacgtcttctcatgctccg | |
| | tgatgcatgaggctctgcacaaccactacacgcagaagagcctctccctgtctccgggtgg | |
| | gtacccatacgatgttccagattacgcttga | |
| Ritux HC knobs | atgggatggtcatgtatcatcctttttctagtagcaactgcaaccggtgtacattcccaggtac | |
| LALA P329G | aactgcagcagcctggggctgagctggtgaagcctggggcctcagtgaagatgtcctgca | |
| 6xHis | aggcttctggctacacatttaccagttacaatatgcactgggtaaaacagacacctggtcgg | 116 |
| | ggcctggaatggattggagctatttatcccggaaatggtgatacttcctacaatcagaagttc | |
| | aaaggcaaggccacattgactgcagacaaatcctccagcacagcctacatgcagctcag | |
| | cagcctgacatctgaggactctgcggtctattactgtgcaagatcgacttactacggcggtga | |
| | ctggtacttcaatgtctggggcgcagggaccacggtcaccgtctctgcagctagcaccaag | |
| | ggcccatcggtcttccccctggcaccctcctccaagagcacctctgggggcacagcggcc | |
| | ctgggctgcctggtcaaggactacttccccgaaccggtgacggtgtcgtggaactcaggcg | |
| | ccctgaccagcggcgtgcacaccttcccggctgtcctacagtcctcaggactctactccctc | |
| | agcagcgtggtgaccgtgccctccagcagcttgggcacccagacctacatctgcaacgtg | |
| | aatcacaagcccagcaacaccaaggtggacaagaaagcagagcccaaatcttgtgaca | |
| | aaactcacacatgcccaccgtgcccagcacctgaagcagccgggggaccgtcagtcttc | |
| | ctcttccccccaaaacccaaggacaccctcatgatctcccggacccctgaggtcacatgcg | |
| | tggtggtggacgtgagccacgaagaccctgaggtcaagttcaactggtacgtggacggcg | |
| | tggaggtgcataatgccaagacaaagccgcgggaggagcagtacaacagcacgtacc | |
| | gtgtggtcagcgtcctcaccgtcctgcaccaggactggctgaatggcaaggagtacaagtg | |
| | caaggtctccaacaaagccctcggagcccccatcgagaaaaccatctccaaagccaaa | |
| | gggcagccccgagaaccacaggtgtacaccctgcccccatgccgggatgagctgacca | |
| | agaaccaggtcagcctgtggtgcctggtcaaaggcttctatcccagcgacatcgccgtgga | |
| | gtgggagagcaatgggcagccggagaacaactacaagaccacgcctcccgtgctggac | |
| | tccgacggctccttcttcctctacagcaagctcaccgtggacaagagcaggtggcagcag | |
| | gggaacgtcttctcatgctccgtgatgcatgaggctctgcacaaccactacacgcagaaga | |
| | gcctctccctgtctccgggtgggcatcaccaccatcaccattga | |
| Tafasitamab HC | atgggatggtcatgtatcatcctttttctagtagcaactgcaaccggtgtacattccgaagttca | 117 |
| knobs LALA | actggtggagagcggcggggggcttgtgaaaccaggcgggtccctgaaactgagctgtg | |
| P329G 6xHis | ctgcgagcggctacaccttcacaagctacgttatgcattgggtgaggcaggcacctggtaa | |
| | gggtcttgagtggatcggctatataaatccctacaatgatggcacgaagtacaacgagaaa | |
| | ttccaagggcgcgtgactatttctagcgataagtcaatctctactgcctatatggaactttcttctt | |
| | tgagaagcgaagacacagctatgtactactgcgcccgaggcacctactactatggaaccc | |
| | gcgtatttgattattgggggcaaggcaccttggtgacagtaagctcagctagcaccaaggg | |
| | cccatcggtcttccccctggcaccctcctccaagagcacctctgggggcacagcggccctg | |
| | ggctgcctggtcaaggactacttccccgaaccggtgacggtgtcgtggaactcaggcgcc | |
| | ctgaccagcggcgtgcacaccttcccggctgtcctacagtcctcaggactctactccctcag | |
| | cagcgtggtgaccgtgccctccagcagcttgggcacccagacctacatctgcaacgtgaat | |
| | cacaagcccagcaacaccaaggtggacaagaaagttgagcccaaatcttgtgacaaaa | |
| | ctcacacatgcccaccgtgcccagcacctgaagcagccgggggaccgtcagtcttcctctt | |
| | ccccccaaaacccaaggacaccctcatgatctcccggacccctgaggtcacatgcgtggt | |
| | ggtggacgtgagccacgaagaccctgaggtcaagttcaactggtacgtggacggcgtgg | |
| | aggtgcataatgccaagacaaagccgcgggaggagcagtacaacagcacgtaccgtgt | |
| | ggtcagcgtcctcaccgtcctgcaccaggactggctgaatggcaaggagtacaagtgcaa | |
| | ggtctccaacaaagccctcggagcccccatcgagaaaaccatctccaaagccaaaggg | |
| | cagccccgagaaccacaggtgtacaccctgcccccatgccgggatgagctgaccaaga | |
| | accaggtcagcctgtggtgcctggtcaaaggcttctatcccagcgacatcgccgtggagtg | |
| | ggagagcaatgggcagccggagaacaactacaagaccacgcctcccgtgctggactcc | |
| | gacggctccttcttcctctacagcaagctcaccgtggacaagagcaggtggcagcagggg | |
| | aacgtcttctcatgctccgtgatgcatgaggctctgcacaaccactacacgcagaagagcc | |
| | tctccctgtctccggggggcatcaccaccatcaccattga | |
| Tafasitamab LC | atgggatggtcatgtatcatcctttttctagtagcaactgcaaccggtgtacattccgatatcgt | 118 |
| | aatgacgcaatccccagcgacgctcagtctcagccccggcgaacgggcgactctttcatgt | |
| | aggagctccaaaagcctccagaatgtcaacggtaatacgtacctttactggtttcaacaga | |
| | agcctggtcagagtccacaacttctgatataccgcatgtctaatctgaatagtggcgtcccgg | |
| | acagattttctggaagcggcagcggaaccgaatttactctcacgatcagttcactcgaacct | |
| | gaggacttcgccgtttactactgtatgcaacatctcgagtatcctatcacgtttggagctggga | |
| | cgaaattggaaatcaagcgcaccgtcgccgcgcccagcgtgttcatcttcccgcccagcg | |
| | acgagcagctgaagagcggcacggccagcgtggtctgcctgctcaacaacttctaccccc | |
| | gggaggccaaggtgcagtggaaggtggacaacgccctgcagtcggggaacagccagg | |
| | agagcgtcaccgagcaggacagcaaggacagcacctacagcctgtcgagcaccctcac | |
| | gctgagcaaggccgactacgagaagcacaaggtgtacgcgtgcgaggtgacccaccag | |
| | ggcctgagcagccccgtcaccaagtcgttcaaccgcggagaatgctga | |
| HuB6H12 HC | atgggatggtcatgtatcatcctttttctagtagcaactgcaaccggtgtacattccgaggtgc | 119 |
| knobs LALA | agctggtggagtctgggggaggcttggtccagcctggggggtccctgagactctcctgtgc | |
| P329G 6xHis | agcctctggattcacctttagtggctatggcatgagctgggtccgccaggctccagggaagg | |
| | ggctggagtgggtggccaccataactagtggtggaacttacacctactatccagactctgtg | |
| | aagggccgattcaccatctccagagacaacgccaagaactcactgtatctgcaaatgaac | |
| | agcctgagagccgaggacacggctgtgtattactgtgcgagatccctcgcgggaaatgct | |
| | atggactactggggccaaggaaccctggtcaccgtctcctcagctagcaccaagggccca | |
| | tcggtcttccccctggcaccctcctccaagagcacctctgggggcacagcggccctgggct | |
| | gcctggtcaaggactacttccccgaaccggtgacggtgtcgtggaactcaggcgccctga | |
| | ccagcggcgtgcacaccttcccggctgtcctacagtcctcaggactctactccctcagcagc | |
| | gtggtgaccgtgccctccagcagcttgggcacccagacctacatctgcaacgtgaatcaca | |
| | agcccagcaacaccaaggtggacaagaaagttgagcccaaatcttgtgacaaaactcac | |
| | acatgcccaccgtgcccagcacctgaagcagccgggggaccgtcagtcttcctcttccccc | |
| | caaaacccaaggacaccctcatgatctcccggacccctgaggtcacatgcgtggtggtgg | |
| | acgtgagccacgaagaccctgaggtcaagttcaactggtacgtggacggcgtggaggtg | |
| | cataatgccaagacaaagccgcgggaggagcagtacaacagcacgtaccgtgtggtca | |
| | gcgtcctcaccgtcctgcaccaggactggctgaatggcaaggagtacaagtgcaaggtct | |
| | ccaacaaagccctcggagcccccatcgagaaaaccatctccaaagccaaagggcagc | |
| | cccgagaaccacaggtgtacaccctgcccccatgccgggatgagctgaccaagaacca | |
| | ggtcagcctgtggtgcctggtcaaaggcttctatcccagcgacatcgccgtggagtgggag | |
| | agcaatgggcagccggagaacaactacaagaccacgcctcccgtgctggactccgacg | |
| | gctccttcttcctctacagcaagctcaccgtggacaagagcaggtggcagcaggggaacg | |
| | tcttctcatgctccgtgatgcatgaggctctgcacaaccactacacgcagaagagcctctcc | |
| | ctgtctccgggtgggcatcaccaccatcaccattga | |
| HuB6H12 LC | ATGGGATGGTCATGTATCATCCTTTTTCTAGTAGCAACTGCAA | 120 |
| | CCGGTGTACATTCCgaaattgtgttgacacagtctccagccaccctgtctttgtctcc | |
| | aggggaaagagccaccctctcctgcagggccagtcagactattagcgactacttacactg | |
| | gtaccaacagaaacctggccaggctcccaggctcctcatcaaatttgcatcccaatccattt | |
| | ctggcatcccagccaggttcagtggcagtgggtctgggacagacttcactctcaccatcag | |
| | cagcctagagcctgaagattttgcagtttattactgtcagaatggtcacggctttcctcggacg | |
| | ttcggccaagggaccaaggtggaaatcaaaCGCACCGTCGCCGCGCCCA | |
| | GCGTGTTCATCTTCCCGCCCAGCGACGAGCAGCTGAAGAGC | |
| | GGCACGGCCAGCGTGGTCTGCCTGCTCAACAACTTCTACCCC | |
| | CGGGAGGCCAAGGTGCAGTGGAAGGTGGACAACGCCCTGCA | |
| | GTCGGGGAACAGCCAGGAGAGCGTCACCGAGCAGGACAGCA | |
| | AGGACAGCACCTACAGCCTGTCGAGCACCCTCACGCTGAGC | |
| | AAGGCCGACTACGAGAAGCACAAGGTGTACGCGTGCGAGGT | |
| | GACCCACCAGGGCCTGAGCAGCCCCGTCACCAAGTCGTTCA | |
| | ACCGCGGAGAATGCTGA | |
| Hu5F9 HC knobs | atgggatggtcatgtatcatcctttttctagtagcaactgcaaccggtgtacattcccaagttca | 121 |
| LALA P329G | acttcaacaacccggcgcagagctggttaagcctggggcgagtgtaatgatgagttgcaa | |
| 6xHis | agcaagcggatacacgtttaccaactacaatatgcattgggtaaaacagacgcctgggca | |
| | aggtcttgaatggatcggcaccatctaccctggcaacgatgatacgtcatacaaccagaag | |
| | tttaaggataaggccacgctgaccgccgataaatcctcctctgcagcgtacatgcagttgtct | |
| | agtcttacgtctgaggactctgcggtgtactattgcgccagggggggttatagagctatggac | |
| | tactgggggcaagggacctccgtgaccgtatcctcagctagcaccaagggcccatcggtc | |
| | ttccccctggcaccctcctccaagagcacctctgggggcacagcggccctgggctgcctgg | |
| | tcaaggactacttccccgaaccggtgacggtgtcgtggaactcaggcgccctgaccagcg | |
| | gcgtgcacaccttcccggctgtcctacagtcctcaggactctactccctcagcagcgtggtg | |
| | accgtgccctccagcagcttgggcacccagacctacatctgcaacgtgaatcacaagccc | |
| | agcaacaccaaggtggacaagaaagttgagcccaaatcttgtgacaaaactcacacatg | |
| | cccaccgtgcccagcacctgaagcagccgggggaccgtcagtcttcctcttccccccaaa | |
| | acccaaggacaccctcatgatctcccggacccctgaggtcacatgcgtggtggtggacgt | |
| | gagccacgaagaccctgaggtcaagttcaactggtacgtggacggcgtggaggtgcata | |
| | atgccaagacaaagccgcgggaggagcagtacaacagcacgtaccgtgtggtcagcgt | |
| | cctcaccgtcctgcaccaggactggctgaatggcaaggagtacaagtgcaaggtctccaa | |
| | caaagccctcggagcccccatcgagaaaaccatctccaaagccaaagggcagccccg | |
| | agaaccacaggtgtacaccctgcccccatgccgggatgagctgaccaagaaccaggtc | |
| | agcctgtggtgcctggtcaaaggcttctatcccagcgacatcgccgtggagtgggagagca | |
| | atgggcagccggagaacaactacaagaccacgcctcccgtgctggactccgacggctcc | |
| | ttcttcctctacagcaagctcaccgtggacaagagcaggtggcagcaggggaacgtcttct | |
| | catgctccgtgatgcatgaggctctgcacaaccactacacgcagaagagcctctccctgtct | |
| | ccgggtgggcatcaccaccatcaccattga | |
| Hu5F9 LC | atgggatggtcatgtatcatcctttttctagtagcaactgcaaccggtgtacattccgatgtgttg | 122 |
| | atgacgcaaaccccattgtctctcccagtaagcctgggcgaccaagcgagtatctcttgccg | |
| | ctcatcccaatcaatcgtttattccaatggcaacacatatctgggatggtacctccaaaaacc | |
| | agggcaaagcccaaagctgcttatatataaagtgtcaaatcgatttagcggagtcccggac | |
| | agattttcagggtcagggtccggaacagattttacgttgaaaatctctcgcgtagaagcaga | |
| | ggacctgggggtctatcattgctttcagggtagtcatgtcccatatacctttggcggtggtaca | |
| | aaggtagaaatcaagcgcaccgtcgccgcgcccagcgtgttcatcttcccgcccagcgac | |
| | gagcagctgaagagcggcacggccagcgtggtctgcctgctcaacaacttctacccccgg | |
| | gaggccaaggtgcagtggaaggtggacaacgccctgcagtcggggaacagccaggag | |
| | agcgtcaccgagcaggacagcaaggacagcacctacagcctgtcgagcaccctcacgc | |
| | tgagcaaggccgactacgagaagcacaaggtgtacgcgtgcgaggtgacccaccagg | |
| | gcctgagcagccccgtcaccaagtcgttcaaccgcggagaatgctga | |
| Avelumab HC | ATGGGATGGTCATGTATCATCCTTTTTCTAGTAGCAACTGCAA | 123 |
| knobs 6xHis | CCGGTGTACATTCCGAAGTTCAATTGCTGGAGAGCGGCGGAG | |
| | GTTTGGTCCAGCCCGGCGGTTCTTTGCGACTGTCCTGCGCAG | |
| | CCTCTGGTTTCACATTTTCTTCCTACATTATGATGTGGGTGCG | |
| | CCAGGCTCCTGGAAAGGGACTTGAATGGGTAAGCAGCATTTA | |
| | TCCCTCTGGTGGGATCACTTTCTACGCCGATACCGTGAAGGG | |
| | GAGATTCACTATATCACGAGACAACTCAAAGAATACCCTCTAT | |
| | CTTCAGATGAATAGTTTGCGAGCCGAGGATACGGCTGTCTAC | |
| | TACTGTGCCAGGATCAAGTTGGGTACCGTGACAACTGTTGAT | |
| | TATTGGGGTCAAGGAACTCTCGTAACCGTCAGCAGTgctagcacc | |
| | aagggcccatcggtcttccccctggcaccctcctccaagagcacctctgggggcacagcg | |
| | gccctgggctgcctggtcaaggactacttccccgaaccggtgacggtgtcgtggaactcag | |
| | gcgccctgaccagcggcgtgcacaccttcccggctgtcctacagtcctcaggactctactcc | |
| | ctcagcagcgtggtgaccgtgccctccagcagcttgggcacccagacctacatctgcaac | |
| | gtgaatcacaagcccagcaacaccaaggtggacaagaaagttGAGCCCAAATC | |
| | TTGTGACAAAACTCACACAtgcccaccgtgcccagcacctgaactcctggggg | |
| | gaccgtcagtcttcctcttccccccaaaacccaaggacaccctcatgatctcccggacccct | |
| | gaggtcacatgcgtggtggtggacgtgagccacgaagaccctgaggtcaagttcaactgg | |
| | tacgtggacggcgtggaggtgcataatgccaagacaaagccgcgggaggagcagtaca | |
| | acagcacgtaccgtgtggtcagcgtcctcaccgtcctgcaccaggactggctgaatggca | |
| | aggagtacaagtgcaaggtctccaacaaagccctcccagcccccatcgagaaaaccatc | |
| | tccaaagccaaagggcagccccgagaaccacaggtgtacaccctgcccccatgccggg | |
| | atgagctgaccaagaaccaggtcagcctgTGGtgcctggtcaaaggcttctatcccagc | |
| | gacatcgccgtggagtgggagagcaatgggcagccggagaacaactacaagaccacg | |
| | cctcccgtgctggactccgacggctccttcttcctctacagcaagctcaccgtggacaagag | |
| | caggtggcagcaggggaacgtcttctcatgctccgtgatgcatgaggctctgcacaaccac | |
| | tacacgcagaagagcctctccctgtctccgggtGGGCATCACCACCATCACC | |
| | ATTGA | |
| Avelumab LC | ATGGGATGGTCATGTATCATCCTTTTTCTAGTAGCAACTGCAA | 124 |
| | CCGGTGTACATTCCCAGAGTGCCCTGACGCAGCCTGCTTCTG | |
| | TCTCTGGGAGTCCAGGGCAGTCCATTACCATTAGTTGTACTG | |
| | GTACATCCTCTGACGTGGGTGGGTATAACTATGTTAGTTGGTA | |
| | TCAACAGCATCCCGGTAAAGCTCCCAAACTGATGATCTACGA | |
| | CGTGAGTAATAGGCCCAGCGGTGTTAGTAACAGGTTTTCAGG | |
| | ATCTAAGAGTGGTAACACAGCGTCATTGACTATAAGTGGGCT | |
| | GCAAGCTGAGGATGAAGCTGATTACTACTGCTCATCATATACG | |
| | TCATCAAGTACAAGAGTATTTGGGACCGGTACGAAAGTCACA | |
| | GTCCTTGGACAGCCAAAAGCCAATCCGACAGTAACTTTGTTC | |
| | CCGCCAAGCTCAGAGGAGCTGCAAGCGAATAAAGCAACTCTT | |
| | GTGTGTCTGATAAGTGATTTTTACCCGGGAGCGGTCACTGTG | |
| | GCGTGGAAGGCAGACGGGTCACCAGTTAAGGCTGGAGTGGA | |
| | GACCACGAAACCGAGCAAGCAAAGCAACAACAAATATGCCGC | |
| | ATCATCATATCTGTCTCTGACCCCCGAGCAGTGGAAGTCCCA | |
| | TCGAAGTTACTCTTGCCAGGTCACCCATGAGGGCTCTACTGT | |
| | TGAGAAGACGGTCGCTCCCACGGAGTGTTAA | |
| Atezolizumab HC | atgggatggtcatgtatcatcctttttctagtagcaactgcaaccggtgtacattccgaagtgc | 125 |
| knobs 6xHis | aactggtcgagtcagggggcgggcttgttcagccggggggtagcttgcgactgtcctgtgc | |
| | cgcttcaggattcacattcagcgattcctggattcattgggtccggcaagccccagggaagg | |
| | gactcgaatgggtggcatggatttctccttatggcggtagcacctactatgcggactcagtga | |
| | aaggacgctttacgatatcagcagatacttcaaaaaatacggcgtatcttcaaatgaattca | |
| | ctccgagctgaggatactgccgtgtattactgcgcgagacggcattggccgggaggttttga | |
| | ttattggggtcagggaaccctcgttaccgtgtcatcagctagcaccaagggcccatcggtctt | |
| | ccccctggcaccctcctccaagagcacctctgggggcacagcggccctgggctgcctggt | |
| | caaggactacttccccgaaccggtgacggtgtcgtggaactcaggcgccctgaccagcg | |
| | gcgtgcacaccttcccggctgtcctacagtcctcaggactctactccctcagcagcgtggtg | |
| | accgtgccctccagcagcttgggcacccagacctacatctgcaacgtgaatcacaagccc | |
| | agcaacaccaaggtggacaagaaagttgagcccaaatcttgtgacaaaactcacacatg | |
| | cccaccgtgcccagcacctgaagcagccgggggaccgtcagtcttcctcttccccccaaa | |
| | acccaaggacaccctcatgatctcccggacccctgaggtcacatgcgtggtggtggacgt | |
| | gagccacgaagaccctgaggtcaagttcaactggtacgtggacggcgtggaggtgcata | |
| | atgccaagacaaagccgcgggaggagcagtacaacagcacgtaccgtgtggtcagcgt | |
| | cctcaccgtcctgcaccaggactggctgaatggcaaggagtacaagtgcaaggtctccaa | |
| | caaagccctcggagcccccatcgagaaaaccatctccaaagccaaagggcagccccg | |
| | agaaccacaggtgtacaccctgcccccatgccgggatgagctgaccaagaaccaggtc | |
| | agcctgtggtgcctggtcaaaggcttctatcccagcgacatcgccgtggagtgggagagca | |
| | atgggcagccggagaacaactacaagaccacgcctcccgtgctggactccgacggctcc | |
| | ttcttcctctacagcaagctcaccgtggacaagagcaggtggcagcaggggaacgtcttct | |
| | catgctccgtgatgcatgaggctctgcacaaccactacacgcagaagagcctctccctgtct | |
| | ccgggtgggcatcaccaccatcaccattga | |
| Atezolizumab LC | atgggatggtcatgtatcatcctttttctagtagcaactgcaaccggtgtacattccgacatac | 126 |
| | aaatgacccaaagtccaagctctctttcagcatcagttggagatcgcgttactatcacctgcc | |
| | gcgcgagtcaagacgtaagcactgctgtagcatggtatcaacagaagccagggaaggc | |
| | cccaaagctcctgatatacagcgccagtttcctgtatagtggtgtacccagccgattttctggc | |
| | agtggaagtggcacggactttacactcaccatcagtagcttgcagccagaagattttgcga | |
| | cgtattactgtcaacaatatttgtaccacccagcgacttttgggcagggaaccaaggtcgag | |
| | ataaagcgcaccgtcgccgcgcccagcgtgttcatcttcccgcccagcgacgagcagctg | |
| | aagagcggcacggccagcgtggtctgcctgctcaacaacttctacccccgggaggccaa | |
| | ggtgcagtggaaggtggacaacgccctgcagtcggggaacagccaggagagcgtcac | |
| | cgagcaggacagcaaggacagcacctacagcctgtcgagcaccctcacgctgagcaa | |
| | ggccgactacgagaagcacaaggtgtacgcgtgcgaggtgacccaccagggcctgagc | |
| | agccccgtcaccaagtcgttcaaccgcggagaatgctga | |
| Siglec-2FL Holes | atgcatctgctggggccctggctgctgctgctggtgctggagtacttggctttctctgactcaag | 127 |
| LALA P329G Fc | taaatgggtttttgagcaccctgaaaccctctacgcctgggagggggcctgcgtctggatcc | |
| HA | cctgcacctacagagccctagatggtgacctggaaagcttcatcctgttccacaatcctgag | |
| | tataacaagaacacctcgaagtttgatgggacaagactctatgaaagcacaaaggatgg | |
| | gaaggttccttctgagcagaaaagggtgcaattcctgggagacaagaataagaactgcac | |
| | actgagtatccacccggtgcacctcaatgacagtggtcagctggggctgaggatggagtcc | |
| | aagactgagaaatggatggaacgaatacacctcaatgtctctgaaaggccttttccacctc | |
| | atatccagctccctccagaaattcaagagtcccaggaagtcactctgacctgcttgctgaatt | |
| | tctcctgctatgggtatccgatccaattgcagtggctcctagagggggttccaatgaggcag | |
| | gctgctgtcacctcgacctccttgaccatcaagtctgtcttcacccggagcgagctcaagttct | |
| | ccccacagtggagtcaccatgggaagattgtgacctgccagcttcaggatgcagatggga | |
| | agttcctctccaatgacacggtgcagctgaacgtgaagcacaccccgaagttggagatca | |
| | aggtcactcccagtgatgccatagtgagggagggggactctgtgaccatgacctgcgagg | |
| | tcagcagcagcaacccggagtacacgacggtatcctggctcaaggatgggacctcgctg | |
| | aagaagcagaatacattcacgctaaacctgcgcgaagtgaccaaggaccagagtggga | |
| | agtactgctgtcaggtctccaatgacgtgggcccgggaaggtcggaagaagtgttcctgca | |
| | agtgcagtatgccccggaaccttccacggttcagatcctccactcaccggctgtggaggga | |
| | agtcaagtcgagtttctttgcatgtcactggccaatcctcttccaacaaattacacgtggtacc | |
| | acaatgggaaagaaatgcagggaaggacagaggagaaagtccacatcccaaagatcc | |
| | tcccctggcacgctgggacttattcctgtgtggcagaaaacattcttggtactggacagagg | |
| | ggcccgggagctgagctggatgtccagtatcctcccaagaaggtgaccacagtgattcaa | |
| | aaccccatgccgattcgagaaggagacacagtgaccctttcctgtaactacaattccagta | |
| | accccagtgttacccggtatgaatggaaaccccatggcgcctgggaggagccatcgcttg | |
| | gggtgctgaagatccaaaacgttggctgggacaacacaaccatcgcctgcgcaaggtgt | |
| | aatagttggtgctcgtgggcctcccctgtcgccctgaatgtccagtatgccccccgagacgtg | |
| | agggtccggaaaatcaagcccctttccgagattcactctggaaactcggtcagcctccaat | |
| | gtgacttctcaagcagccaccccaaagaagtccagttcttctgggagaaaaatggcaggct | |
| | tctggggaaagaaagccagctgaattttgactccatctccccagaagatgctgggagttac | |
| | agctgctgggtgaacaactccataggacagacagcgtccaaggcctggacacttgaagt | |
| | gctgtatgcacccaggaggctgcgtgtgtccatgagcccgggggaccaagtgatggagg | |
| | ggaagagtgcaaccctgacctgtgagagcgacgccaaccctcccgtctcccactacacct | |
| | ggtttgactggaataaccaaagcctcccctaccacagccagaagctgagattggagccgg | |
| | tgaaggtccagcactcgggtgcctactggtgccaggggaccaacagtgtgggcaagggc | |
| | cgttcgcctctcagcaccctcaccgtctactatagcccggagaccatcggcaggcgaggc | |
| | ggaggtggaggccccaaatcttgtgacaaaactcacacatgcccaccgtgcccagcacct | |
| | gaagcagccgggggaccgtcagtcttcctcttccccccaaaacccaaggacaccctcatg | |
| | atctcccggacccctgaggtcacatgcgtggtggtggacgtgagccacgaagaccctgag | |
| | gtcaagttcaactggtacgtggacggcgtggaggtgcataatgccaagacaaagccgcg | |
| | ggaggagcagtacaacagcacgtaccgtgtggtcagcgtcctcaccgtcctgcaccagg | |
| | actggctgaatggcaaggagtacaagtgcaaggtctccaacaaagccctcggagccccc | |
| | atcgagaaaaccatctccaaagccaaagggcagccccgagaaccacaggtgtgcacc | |
| | ctgcccccatcccgggatgagctgaccaagaaccaggtcagcctgagctgcgcggtcaa | |
| | aggcttctatcccagcgacatcgccgtggagtgggagagcaatgggcagccggagaac | |
| | aactacaagaccacgcctcccgtgctggactccgacggctccttcttcctcgtgagcaagct | |
| | caccgtggacaagagcaggtggcagcaggggaacgtcttctcatgctccgtgatgcatga | |
| | ggctctgcacaaccactacacgcagaagagcctctccctgtctccggggggtacccatac | |
| | gatgttccagattacgcttga | |
| Siglec-2Ig2 Holes | atgcatctgctggggccctggctgctgctgctggtgctggagtacttggctttctctgactcaag | 128 |
| LALA P329G Fc | taaatgggtttttgagcaccctgaaaccctctacgcctggggggggcctgcgtctggatcc | |
| HA | cctgcacctacagagccctagatggtgacctggaaagcttcatcctgttccacaatcctgag | |
| | tataacaagaacacctcgaagtttgatgggacaagactctatgaaagcacaaaggatgg | |
| | gaaggttccttctgagcagaaaagggtgcaattcctgggagacaagaataagaactgcac | |
| | actgagtatccacccggtgcacctcaatgacagtggtcagctggggctgaggatggagtcc | |
| | aagactgagaaatggatggaacgaatacacctcaatgtctctgaaaggccttttccacctc | |
| | atatccagctccctccagaaattcaagagtcccaggaagtcactctgacctgcttgctgaatt | |
| | tctcctgctatgggtatccgatccaattgcagtggctcctagagggggttccaatgaggcag | |
| | gctgctgtcacctcgacctccttgaccatcaagtctgtcttcacccggagcgagctcaagttct | |
| | ccccacagtggagtcaccatgggaagattgtgacctgccagcttcaggatgcagatggga | |
| | agttcctctccaatgacacggtgcagctgaacgtgaagcacaccccgaagttggagatca | |
| | aggtcactcccagtgatgccatagtgagggagggggactctgtgaccatgacctgcgagg | |
| | tcagcagcagcaacccggagtacacgacggtatcctggctcaaggatgggacctcgctg | |
| | aagaagcagaatacattcacgctaaacctgcgcgaagtgaccaaggaccagagtggga | |
| | agtactgctgtcaggtctccaatgacgtgggcccgggaaggtcggaagaagtgttcctgca | |
| | aggcggaggtggaggccccaaatcttgtgacaaaactcacacatgcccaccgtgcccag | |
| | cacctgaagcagccgggggaccgtcagtcttcctcttccccccaaaacccaaggacaccc | |
| | tcatgatctcccggacccctgaggtcacatgcgtggtggtggacgtgagccacgaagacc | |
| | ctgaggtcaagttcaactggtacgtggacggcgtggaggtgcataatgccaagacaaagc | |
| | cgcgggaggagcagtacaacagcacgtaccgtgtggtcagcgtcctcaccgtcctgcacc | |
| | aggactggctgaatggcaaggagtacaagtgcaaggtctccaacaaagccctcggagc | |
| | ccccatcgagaaaaccatctccaaagccaaagggcagccccgagaaccacaggtgtgc | |
| | accctgcccccatcccgggatgagctgaccaagaaccaggtcagcctgagctgcgcggt | |
| | caaaggcttctatcccagcgacatcgccgtggagtgggagagcaatgggcagccggag | |
| | aacaactacaagaccacgcctcccgtgctggactccgacggctccttcttcctcgtgagca | |
| | agctcaccgtggacaagagcaggtggcagcaggggaacgtcttctcatgctccgtgatgc | |
| | atgaggctctgcacaaccactacacgcagaagagcctctccctgtctccggggggtaccc | |
| | atacgatgttccagattacgcttga | |
| Siglec-10Ig2 | atgctactgccactgctgctgtcctcgctgctgggcgggtcccaggctatggatgggagattc | 129 |
| Holes LALA | tggatacgagtgcaggagtcagtgatggtgccggagggcctgtgcatctctgtgccctgctc | |
| P329G Fc HA | tttctcctacccccgacaggactggacagggtctaccccagcttatggctactggttcaaagc | |
| | agtgactgagacaaccaagggtgctcctgtggccacaaaccaccagagtcgagaggtg | |
| | gaaatgagcacccggggccgattccagctcactggggatcccgccaaggggaactgctc | |
| | cttggtgatcagagacgcgcagatgcaggatgagtcacagtacttctttcgggtggagaga | |
| | ggaagctatgtgagatataatttcatgaacgatgggttctttctaaaagtaacagccctgactc | |
| | agaagcctgatgtctacatccccgagaccctggagcccgggcagccggtgacggtcatct | |
| | gtgtgtttaactgggcctttgaggaatgtccacccccttctttctcctggacgggggctgccctc | |
| | tcctcccaaggaaccaaaccaacgacctcccacttctcagtgctcagcttcacgcccagac | |
| | cccaggaccacaacaccgacctcacctgccatgtggacttctccagaaagggtgtgagcg | |
| | cacagaggaccgtccgactccgtgtggcctatgcccccagagaccttgttatcagcatttca | |
| | cgtgacaacacgccagccctggagccccagccccagggaaatgtcccatacctggaag | |
| | cccaaaaaggccagttcctgcggctcctctgtgctgctgacagccagccccctgccacact | |
| | gagctgggtcctgcagaacagagtcctctcctcgtcccatccctggggccctagacccctg | |
| | gggctggagctgcccggggtgaaggctggggattcagggcgctacacctgccgagcgg | |
| | agaacaggcttggctcccagcagcgagccctggacctctctggcggaggtggaggcccc | |
| | aaatcttgtgacaaaactcacacatgcccaccgtgcccagcacctgaagcagccggggg | |
| | accgtcagtcttcctcttccccccaaaacccaaggacaccctcatgatctcccggacccctg | |
| | aggtcacatgcgtggtggtggacgtgagccacgaagaccctgaggtcaagttcaactggt | |
| | acgtggacggcgtggaggtgcataatgccaagacaaagccgcgggaggagcagtaca | |
| | acagcacgtaccgtgtggtcagcgtcctcaccgtcctgcaccaggactggctgaatggca | |
| | aggagtacaagtgcaaggtctccaacaaagccctcggagcccccatcgagaaaaccat | |
| | ctccaaagccaaagggcagccccgagaaccacaggtgtgcaccctgcccccatcccgg | |
| | gatgagctgaccaagaaccaggtcagcctgagctgcgcggtcaaaggcttctatcccagc | |
| | gacatcgccgtggagtgggagagcaatgggcagccggagaacaactacaagaccacg | |
| | cctcccgtgctggactccgacggctccttcttcctcgtgagcaagctcaccgtggacaagag | |
| | caggtggcagcaggggaacgtcttctcatgctccgtgatgcatgaggctctgcacaaccac | |
| | tacacgcagaagagcctctccctgtctccggggggtacccatacgatgttccagattacgc | |
| | ttga | |
| ESelSushi2 | atgattgcttcacagtttctctcagctctcactttggtgcttctcattaaagagagtggagcctgg | 130 |
| LALAP329G | tcttacaacacctccacggaagctatgacttatgatgaggccagtgcttattgtcagcaaag | |
| Holes Fc HA | gtacacacacctggttgcaattcaaaacaaagaagagattgagtacctaaactccatattg | |
| | agctattcaccaagttattactggattggaatcagaaaagtcaacaatgtgtgggtctgggta | |
| | ggaacccagaaacctctgacagaagaagccaagaactgggctccaggtgaacccaac | |
| | aataggcaaaaagatgaggactgcgtggagatctacatcaagagagaaaaagatgtgg | |
| | gcatgtggaatgatgagaggtgcagcaagaagaagcttgccctatgctacacagctgcct | |
| | gtaccaatacatcctgcagtggccacggtgaatgtgtagagaccatcaataattacacttgc | |
| | aagtgtgaccctggcttcagtggactcaagtgtgagcaaattgtgaactgtacagccctgga | |
| | atcccctgagcatggaagcctggtttgcagtcacccactgggaaacttcagctacaattcttc | |
| | ctgctctatcagctgtgataggggttacctgccaagcagcatggagaccatgcagtgtatgt | |
| | cctctggagaatggagtgctcctattccagcctgcaatgtggttgagtgtgatgctgtgacaa | |
| | atccagccaatgggttcgtggaatgtttccaaaaccctggaagcttcccatggaacacaac | |
| | ctgtacatttgactgtgaagaaggatttgaactaatgggagcccagagccttcagtgtacctc | |
| | atctgggaattgggacaacgagaagccaacgtgtaaagctggcggaggtggaggcccc | |
| | aaatcttgtgacaaaactcacacatgcccaccgtgcccagcacctgaagcagccggggg | |
| | accgtcagtcttcctcttccccccaaaacccaaggacaccctcatgatctcccggacccctg | |
| | aggtcacatgcgtggtggtggacgtgagccacgaagaccctgaggtcaagttcaactggt | |
| | acgtggacggcgtggaggtgcataatgccaagacaaagccgcgggaggagcagtaca | |
| | acagcacgtaccgtgtggtcagcgtcctcaccgtcctgcaccaggactggctgaatggca | |
| | aggagtacaagtgcaaggtctccaacaaagccctcggagcccccatcgagaaaaccat | |
| | ctccaaagccaaagggcagccccgagaaccacaggtgtgcaccctgcccccatcccgg | |
| | gatgagctgaccaagaaccaggtcagcctgagctgcgcggtcaaaggcttctatcccagc | |
| | gacatcgccgtggagtgggagagcaatgggcagccggagaacaactacaagaccacg | |
| | cctcccgtgctggactccgacggctccttcttcctcgtgagcaagctcaccgtggacaagag | |
| | caggtggcagcaggggaacgtcttctcatgctccgtgatgcatgaggctctgcacaaccac | |
| | tacacgcagaagagcctctccctgtctccggggggtacccatacgatgttccagattacgc | |
| | ttga | |
| PSelSushi2 | atggccaactgccaaatagccatcttgtaccagagattccagagagtggtctttggaatttcc | 131 |
| LALAP329G | caactcctttgcttcagtgccctgatctctgaactaacaaaccagaaagaagtggcagcatg | |
| Holes Fc HA | gacttatcattacagcacaaaagcatactcatggaatatttcccgtaaatactgccagaatc | |
| | gctacacagacttagtggccatccagaataaaaatgaaattgattacctcaataaggtccta | |
| | ccctactacagctcctactactggattgggatccgaaagaacaataagacatggacatgg | |
| | gtgggaaccaaaaaggctctcaccaacgaggctgagaactgggctgataatgaacctaa | |
| | caacaaaaggaacaacgaggactgcgtggagatatacatcaagagtccgtcagcccct | |
| | ggcaagtggaatgatgagcactgcttgaagaaaaagcacgcattgtgttacacagcctcct | |
| | gccaggacatgtcctgcagcaaacaaggagagtgcctcgagaccatcgggaactacac | |
| | ctgctcctgttaccctggattctatgggccagaatgtgaatacgtgagagagtgtggagaact | |
| | tgagctccctcaacacgtgctcatgaactgcagccaccctctgggaaacttctcttttaactcg | |
| | cagtgcagcttccactgcactgacgggtaccaagtaaatgggcccagcaagctggaatgc | |
| | ttggcttctggaatctggacaaataagcctccacagtgtttagctgcccagtgcccacccctg | |
| | aagattcctgaacgaggaaacatgacctgccttcattctgcaaaagcattccagcatcagtc | |
| | tagctgcagcttcagttgtgaagagggatttgcattagttggaccggaagtggtgcaatgca | |
| | cagcctcgggggtatggacagccccagccccagtgtgtaaagctggcggaggtggaggc | |
| | cccaaatcttgtgacaaaactcacacatgcccaccgtgcccagcacctgaagcagccgg | |
| | gggaccgtcagtcttcctcttccccccaaaacccaaggacaccctcatgatctcccggacc | |
| | cctgaggtcacatgcgtggtggtggacgtgagccacgaagaccctgaggtcaagttcaac | |
| | tggtacgtggacggcgtggaggtgcataatgccaagacaaagccgcgggaggagcagt | |
| | acaacagcacgtaccgtgtggtcagcgtcctcaccgtcctgcaccaggactggctgaatg | |
| | gcaaggagtacaagtgcaaggtctccaacaaagccctcggagcccccatcgagaaaac | |
| | catctccaaagccaaagggcagccccgagaaccacaggtgtgcaccctgcccccatccc | |
| | gggatgagctgaccaagaaccaggtcagcctgagctgcgcggtcaaaggcttctatccca | |
| | gcgacatcgccgtggagtgggagagcaatgggcagccggagaacaactacaagacca | |
| | cgcctcccgtgctggactccgacggctccttcttcctcgtgagcaagctcaccgtggacaag | |
| | agcaggtggcagcaggggaacgtcttctcatgctccgtgatgcatgaggctctgcacaacc | |
| | actacacgcagaagagcctctccctgtctccggggggtacccatacgatgttccagattac | |
| | gcttga | |
| Teplizumab HC | atgggatggtcatgtatcatcctttttctagtagcaactgcaaccggtgtacattcccaagttca | 132 |
| LALA knobs | actggtccagagtggaggcggagtagtgcaacccggtaggtcactccggctgagttgtaa | |
| 6xHis | ggcttcagggtatactttcacgcgctacacgatgcactgggtacgacaagcccccggaaa | |
| | agggctggaatggataggctacatcaatccgagtagaggctacacaaactataatcagaa | |
| | ggtcaaagataggtttactatttcccgcgataacagcaaaaatacggcttttctccaaatgga | |
| | ctctttgaggcccgaagacactggggtttatttttgtgccaggtattacgatgaccactattgttt | |
| | ggactactggggacagggcaccccagtcacggttagcagtgctagcaccaagggcccat | |
| | cggtcttccccctggcaccctcctccaagagcacctctgggggcacagcggccctgggctg | |
| | cctggtcaaggactacttccccgaaccggtgacggtgtcgtggaactcaggcgccctgacc | |
| | agcggcgtgcacaccttcccggctgtcctacagtcctcaggactctactccctcagcagcgt | |
| | ggtgaccgtgccctccagcagcttgggcacccagacctacatctgcaacgtgaatcacaa | |
| | gcccagcaacaccaaggtggacaagaaagttgagcccaaatcttgtgacaaaactcaca | |
| | catgcccaccgtgcccagcacctgaagcagccgggggaccgtcagtcttcctcttcccccc | |
| | aaaacccaaggacaccctcatgatctcccggacccctgaggtcacatgcgtggtggtgga | |
| | cgtgagccacgaagaccctgaggtcaagttcaactggtacgtggacggcgtggaggtgc | |
| | ataatgccaagacaaagccgcgggaggagcagtacaacagcacgtaccgtgtggtcag | |
| | cgtcctcaccgtcctgcaccaggactggctgaatggcaaggagtacaagtgcaaggtctc | |
| | caacaaagccctcggagcccccatcgagaaaaccatctccaaagccaaagggcagcc | |
| | ccgagaaccacaggtgtacaccctgcccccatgccgggatgagctgaccaagaaccag | |
| | gtcagcctgtggtgcctggtcaaaggcttctatcccagcgacatcgccgtggagtgggaga | |
| | gcaatgggcagccggagaacaactacaagaccacgcctcccgtgctggactccgacgg | |
| | ctccttcttcctctacagcaagctcaccgtggacaagagcaggtggcagcaggggaacgt | |
| | cttctcatgctccgtgatgcatgaggctctgcacaaccactacacgcagaagagcctctccc | |
| | tgtctccggggggcatcaccaccatcaccattga | |
| Teplizumab LC | atgggatggtcatgtatcatcctttttctagtagcaactgcaaccggtgtacattccgatataca | 133 |
| | aatgacccaatcaccatctagcctctctgcctcagtaggtgaccgggttacgataacctgttc | |
| | tgcctcctctagcgtctcctatatgaattggtatcaacaaacaccaggcaaagcgcccaaa | |
| | cgatggatctacgacacgtccaagttggcatctggagtgccctcacgcttctcaggaagcg | |
| | gatcagggacggattacacctttaccattagcagcctgcaaccagaggacattgcaacttat | |
| | tattgccagcaatggtcatcaaatccattcaccttcggtcaaggcactaagctccagataact | |
| | cgcaccgtcgccgcgcccagcgtgttcatcttcccgcccagcgacgagcagctgaagag | |
| | cggcacggccagcgtggtctgcctgctcaacaacttctacccccgggaggccaaggtgca | |
| | gtggaaggtggacaacgccctgcagtcggggaacagccaggagagcgtcaccgagca | |
| | ggacagcaaggacagcacctacagcctgtcgagcaccctcacgctgagcaaggccgac | |
| | tacgagaagcacaaggtgtacgcgtgcgaggtgacccaccagggcctgagcagccccg | |
| | tcaccaagtcgttcaaccgcggagaatgctga | |
| Teplizumab HC | atgggatggtcatgtatcatcctttttctagtagcaactgcaaccggtgtacattcccaagttca | 134 |
| LALA knobs | actggtccagagtggaggcggagtagtgcaacccggtaggtcactccggctgagttgtaa | |
| 6xHis | ggcttcagggtatactttcacgcgctacacgatgcactgggtacgacaagcccccggaaa | |
| | agggctggaatggataggctacatcaatccgagtagaggctacacaaactataatcagaa | |
| | ggtcaaagataggtttactatttcccgcgataacagcaaaaatacggcttttctccaaatgga | |
| | ctctttgaggcccgaagacactggggtttatttttgtgccaggtattacgatgaccactattgttt | |
| | ggactactggggacagggcaccccagtcacggttagcagtgctagcaccaagggcccat | |
| | cggtcttccccctggcaccctcctccaagagcacctctgggggcacagcggccctgggctg | |
| | cctggtcaaggactacttccccgaaccggtgacggtgtcgtggaactcaggcgccctgacc | |
| | agcggcgtgcacaccttcccggctgtcctacagtcctcaggactctactccctcagcagcgt | |
| | ggtgaccgtgccctccagcagcttgggcacccagacctacatctgcaacgtgaatcacaa | |
| | gcccagcaacaccaaggtggacaagaaagttgagcccaaatcttgtgacaaaactcaca | |
| | catgcccaccgtgcccagcacctgaagcagccgggggaccgtcagtcttcctcttcccccc | |
| | aaaacccaaggacaccctcatgatctcccggacccctgaggtcacatgcgtggtggtgga | |
| | cgtgagccacgaagaccctgaggtcaagttcaactggtacgtggacggcgtggaggtgc | |
| | ataatgccaagacaaagccgcgggaggagcagtacaacagcacgtaccgtgtggtcag | |
| | cgtcctcaccgtcctgcaccaggactggctgaatggcaaggagtacaagtgcaaggtctc | |
| | caacaaagccctcggagcccccatcgagaaaaccatctccaaagccaaagggcagcc | |
| | ccgagaaccacaggtgtacaccctgcccccatgccgggatgagctgaccaagaaccag | |
| | gtcagcctgtggtgcctggtcaaaggcttctatcccagcgacatcgccgtggagtgggaga | |
| | gcaatgggcagccggagaacaactacaagaccacgcctcccgtgctggactccgacgg | |
| | ctccttcttcctctacagcaagctcaccgtggacaagagcaggtggcagcaggggaacgt | |
| | cttctcatgctccgtgatgcatgaggctctgcacaaccactacacgcagaagagcctctccc | |
| | tgtctccggggggcatcaccaccatcaccattga | |
| Teplizumab LC | atgggatggtcatgtatcatcctttttctagtagcaactgcaaccggtgtacattccgatataca | 135 |
| | aatgacccaatcaccatctagcctctctgcctcagtaggtgaccgggttacgataacctgttc | |
| | tgcctcctctagcgtctcctatatgaattggtatcaacaaacaccaggcaaagcgcccaaa | |
| | cgatggatctacgacacgtccaagttggcatctggagtgccctcacgcttctcaggaagcg | |
| | gatcagggacggattacacctttaccattagcagcctgcaaccagaggacattgcaacttat | |
| | tattgccagcaatggtcatcaaatccattcaccttcggtcaaggcactaagctccagataact | |
| | cgcaccgtcgccgcgcccagcgtgttcatcttcccgcccagcgacgagcagctgaagag | |
| | cggcacggccagcgtggtctgcctgctcaacaacttctacccccgggaggccaaggtgca | |
| | gtggaaggtggacaacgccctgcagtcggggaacagccaggagagcgtcaccgagca | |
| | ggacagcaaggacagcacctacagcctgtcgagcaccctcacgctgagcaaggccgac | |
| | tacgagaagcacaaggtgtacgcgtgcgaggtgacccaccagggcctgagcagccccg | |
| | tcaccaagtcgttcaaccgcggagaatgctga | |
| Galectin-1 LALA | atgctgctgctgctgctgcccctgctctgggggagggagagggcggaaggacaggcttgt | 136 |
| Holes Fc HA | ggtctggtcgccagcaacctgaatctcaaacctggagagtgccttcgagtgcgaggcgag | |
| | gtggctcctgacgctaagagcttcgtgctgaacctgggcaaagacagcaacaacctgtgc | |
| | ctgcacttcaaccctcgcttcaacgcccacggcgacgccaacaccatcgtgtgcaacagc | |
| | aaggacggcggggcctgggggaccgagcagcgggaggctgtctttcccttccagcctgg | |
| | aagtgttgcagaggtgtgcatcaccttcgaccaggccaacctgaccgtcaagctgccagat | |
| | ggatacgaattcaagttccccaaccgcctcaacctggaggccatcaactacatggcagct | |
| | gacggtgacttcaagatcaaatgtgtggcctttgacggcggaggtggaggccccaaatctt | |
| | gtgacaaaactcacacatgcccaccgtgcccagcacctgaagcagccgggggaccgtc | |
| | agtcttcctcttccccccaaaacccaaggacaccctcatgatctcccggacccctgaggtca | |
| | catgcgtggtggtggacgtgagccacgaagaccctgaggtcaagttcaactggtacgtgg | |
| | acggcgtggaggtgcataatgccaagacaaagccgcgggaggagcagtacaacagca | |
| | cgtaccgtgtggtcagcgtcctcaccgtcctgcaccaggactggctgaatggcaaggagta | |
| | caagtgcaaggtctccaacaaagccctcccagcccccatcgagaaaaccatctccaaag | |
| | ccaaagggcagccccgagaaccacaggtgtgcaccctgcccccatcccgggatgagct | |
| | gaccaagaaccaggtcagcctgagctgcgcggtcaaaggcttctatcccagcgacatcg | |
| | ccgtggagtgggagagcaatgggcagccggagaacaactacaagaccacgcctcccgt | |
| | gctggactccgacggctccttcttcctcgtgagcaagctcaccgtggacaagagcaggtgg | |
| | cagcaggggaacgtcttctcatgctccgtgatgcatgaggctctgcacaaccactacacgc | |
| | agaagagcctctccctgtctccggggggtacccatacgatgttccagattacgcttga | |
| Galectin-3 LALA | atgctgctgctgctgctgcccctgctctgggggagggagagggcggaaggacaggcaga | 137 |
| Holes Fc HA | caatttttcgctccatgatgcgttatctgggtctggaaacccaaaccctcaaggatggcctgg | |
| | cgcatgggggaaccagcctgctggggcagggggctacccaggggcttcctatcctgggg | |
| | cctaccccgggcaggcacccccaggggcttatcctggacaggcacctccaggcgcctac | |
| | catggagcacctggagcttatcccggagcacctgcacctggagtctacccagggccaccc | |
| | agcggccctggggcctacccatcttctggacagccaagtgcccccggagcctaccctgcc | |
| | actggcccctatggcgcccctgctgggccactgattgtgccttataacctgcctttgcctgggg | |
| | gagtggtgcctcgcatgctgataacaattctgggcacggtgaagcccaatgcaaacagaa | |
| | ttgctttagatttccaaagagggaatgatgttgccttccactttaacccacgcttcaatgagaa | |
| | caacaggagagtcattgtttgcaatacaaagctggataataactggggaagggaagaaa | |
| | gacagtcggttttcccatttgaaagtgggaaaccattcaaaatacatgtactggttgaacctg | |
| | accacttcaaggttgcagtgaatgatgctcacttgttgcagtacaatcatcgggttaaaaaac | |
| | tcaatgaaatcagcaaactgggaatttctggtgacatagacctcaccagtgcttcatatacc | |
| | atgataggcggaggtggaggccccaaatcttgtgacaaaactcacacatgcccaccgtgc | |
| | ccagcacctgaagcagccgggggaccgtcagtcttcctcttccccccaaaacccaagga | |
| | caccctcatgatctcccggacccctgaggtcacatgcgtggtggtggacgtgagccacga | |
| | agaccctgaggtcaagttcaactggtacgtggacggcgtggaggtgcataatgccaagac | |
| | aaagccgcgggaggagcagtacaacagcacgtaccgtgtggtcagcgtcctcaccgtcc | |
| | tgcaccaggactggctgaatggcaaggagtacaagtgcaaggtctccaacaaagccctc | |
| | ccagcccccatcgagaaaaccatctccaaagccaaagggcagccccgagaaccacag | |
| | gtgtgcaccctgcccccatcccgggatgagctgaccaagaaccaggtcagcctgagctgc | |
| | gcggtcaaaggcttctatcccagcgacatcgccgtggagtgggagagcaatgggcagcc | |
| | ggagaacaactacaagaccacgcctcccgtgctggactccgacggctccttcttcctcgtg | |
| | agcaagctcaccgtggacaagagcaggtggcagcaggggaacgtcttctcatgctccgtg | |
| | atgcatgaggctctgcacaaccactacacgcagaagagcctctccctgtctccggggggt | |
| | acccatacgatgttccagattacgcttga | |

### REFERENCES

Barkal, A. A., R. E. Brewer, M. Markovic, M. Kowarsky, S. A. Barkal, B. W. Zaro, V. Krishnan, et al. 2019. “CD24 Signalling through Macrophage Siglec-10 Is a Target for Cancer Immunotherapy.” Nature 572: 392-96.
Garcia-Manero, Guillermo, Naval Guastad Daver, Jin Xu, Mark Chao, Trisha Chung, Anderson Tan, Vivian Wang, Andrew Wei, Paresh Vyas, and David Andrew Sallman. 2021. “Magrolimab+Azacitidine versus Azacitidine+Placebo in Untreated Higher Risk (HR) Myelodysplastic Syndrome (MDS): The Phase 3, Randomized, ENHANCE Study.” Journal of Clinical Orthodontics: JCO 39 (15_suppl): TPS7055-TPS7055.
Gray, M. A., M. A. Stanczak, N. R. Mantuano, H. Xiao, J. F. A. Pijnenborg, S. A. Malaker, C. L. Miller, et al. 2020. “Targeted Glycan Degradation Potentiates the Anticancer Immune Response in Vivo.” Nature Chemical Biology 16: 1376-1384.
Ibarlucea-Benitez, Itziar, Polina Weitzenfeld, Patrick Smith, and Jeffrey V. Ravetch. 2021. “Siglecs-7/9 Function as Inhibitory Immune Checkpoints in Vivo and Can Be Targeted to Enhance Therapeutic Antitumor Immunity.” Proceedings of the National Academy of Sciences of the United States of America 118 (26). https://doi.org/10.1073/pnas.2107424118.
Liu, Jie, Lijuan Wang, Feifei Zhao, Serena Tseng, Cyndhavi Narayanan, Lei Shura, Stephen Willingham, et al. 2015. “Pre-Clinical Development of a Humanized Anti-CD47 Antibody with Anti-Cancer Therapeutic Potential.” PloS One 10 (9): e0137345.
Wisnovsky, Simon, Leonhard Möckl, Stacy A. Malaker, Kayvon Pedram, Gaelen T. Hess, Nicholas M. Riley, Melissa A. Gray, et al. 2021. “Genome-Wide CRISPR Screens Reveal a Specific Ligand for the Glycan-Binding Immune Checkpoint Receptor Siglec-7.” Proceedings of the National Academy of Sciences of the United States of America 118 (5). https://doi.org/10.1073/pnas.2015024118.
Yang, Riyao, Linlin Sun, Ching-Fei Li, Yu-Han Wang, Jun Yao, Hui Li, Meisi Yan, et al. 2021. “Galectin-9 Interacts with PD-1 and TIM-3 to Regulate T Cell Death and Is a Target for Cancer Immunotherapy.” Nature Communications 12 (1): 1-17.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A bispecific molecule comprising:
a cell-targeting moiety; and
a glycan-binding moiety.

2. The bispecific molecule of claim 1, wherein the cell-targeting moiety is a cancer cell-targeting moiety.

3. The bispecific molecule of claim 2, wherein the cancer cell-targeting moiety binds to a cancer cell surface molecule selected from the group consisting of: 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD20, CD22, CD25, CD27L, CD30, CD33, CD37, CD44, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvIII, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), GD2 ganglioside, glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), programmed cell death receptor ligand 1 (PD-L1), programmed cell death receptor ligand 2 (PD-L2), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), Tn antigen, trophoblast cell-surface antigen (TROP-2), and Wilms' tumor 1 (WT1).

4. The bispecific molecule of claim 1, wherein the cell-targeting moiety is an immune cell-targeting moiety.

5. The bispecific molecule of claim 4, wherein the immune cell-targeting moiety binds to an immune cell surface molecule selected from the group consisting of: PD-1, PD-L1, PD-L2, CLTA-4, VISTA, LAG-3, TIM-3, CD24, CD47, SIRPalpha, CD3, CD8, CD4, CD28, CD80, CD86, CD19, ICOS, OX40, OX40L, GD3 ganglioside, TIGIT, Siglec-2, Siglec-3, Siglec-7, Siglec-8, Siglec-9, Siglec-10, Siglec-15, galectin-9, B7-H3, B7-H4, CD40, CD40L, B7RP1, CD70, CD27, BTLA, HVEM, KIR, 4-1BB, 4-1BBL, CD226, CD155, CD112, GITR, GITRL, A2aR, CD137, CD137L, CD45, CD206, CD163, TRAIL, NKG2D, CD16, and TGF-beta.

6. The bispecific molecule of any one of claims 1 to 5, wherein the glycan-binding moiety comprises a sialoglycan-binding moiety.

7. The bispecific molecule of claim 6, wherein the sialoglycan-binding moiety comprises the sialoglycan-binding domain of a lectin.

8. The bispecific molecule of claim 7, wherein the lectin is a sialic acid-binding immunoglobulin-like lectin (Siglec).

9. The bispecific molecule of claim 8, wherein the Siglec is a CD33-related Siglec.

10. The bispecific molecule of claim 9, wherein the CD33-related Siglec is selected from the group consisting of: Siglec-7, Siglec-9, and Siglec-10.

11. The bispecific molecule of claim 10, wherein the CD33-related Siglec is Siglec-7.

12. The bispecific molecule of claim 10, wherein the CD33-related Siglec is Siglec-9.

13. The bispecific molecule of claim 8, wherein the Siglec is Siglec-15.

14. The bispecific molecule of claim 6, wherein the sialoglycan-binding moiety comprises the sialoglycan-binding domain of a Siglec-like adhesin.

15. The bispecific molecule of any one of claims 1 to 5, wherein the glycan-binding moiety comprises the glycan-binding domain of a C-type lectin.

16. The bispecific molecule of claim 15, wherein the C-type lectin is DECTIN-1, lectin-like oxidized low-density lipoprotein receptor-1 (LOX-1), C-type lectin-like receptor-1 (CLEC-1), C-type lectin-like receptor 2 (CLEC-2), myeloid inhibitory C-type lectin-like receptor (MICL), CLEC9A, DC immunoreceptor (DCIR), DECTIN-2, blood DC antigen-2 (BDCA-2), macrophage-inducible C-type lectin (MINCLE), macrophage galactose lectin (MGL), or asialoglycoprotein receptor (ASGPR).

17. The bispecific molecule of any one of claims 1 to 5, wherein the glycan-binding moiety comprises the glycan-binding domain of a galectin.

18. The bispecific molecule of claim 17, wherein the galectin is Gal-1, Gal-2, Gal-3, Gal-4, Gal-5, Gal-6, Gal-7, Gal-8, Gal-9, Gal-10, Gal-11, Gal-12, Gal-13, Gal-14, or Gal-15.

19. The bispecific molecule of claim 17, wherein the galectin is Gal-1.

20. The bispecific molecule of claim 17, wherein the galectin is Gal-3.

21. The bispecific molecule of any one of claims 1 to 5, wherein the glycan-binding moiety comprises the glycan-binding domain of a selectin.

22. The bispecific molecule of claim 21, wherein the selectin is P-Selectin (CD62P), E-Selectin (CD62E), or L-Selectin (CD62L).

23. The bispecific molecule of any one of claims 1 to 22, wherein the cell-targeting moiety comprises a ligand for a receptor on the surface of a target cell, or a small molecule that binds to a cell surface molecule on a target cell.

24. The bispecific molecule of any one of claims 1 to 22, wherein the cell-targeting moiety comprises the antigen-binding domain of an antibody.

25. The bispecific molecule of any one of claims 1 to 22, wherein the cell-targeting moiety comprises an antibody heavy chain comprising a variable heavy chain (V_{H}) region and an antibody light chain comprising a variable light chain (V_{L}) region.

26. The bispecific molecule of claim 25, wherein the antibody heavy chain comprises a CH1 domain, a hinge region, a CH2 domain, a CH3 domain, or any combination thereof.

27. The bispecific molecule of claim 25 or claim 26, wherein the antibody heavy chain comprises a CH2 domain, a CH3 domain, or both.

28. The bispecific molecule of any one of claims 25 to 27, wherein the antibody heavy chain comprises a fragment crystallizable (Fc) region.

29. The bispecific molecule of any one of claims 1 to 28, wherein the glycan-binding moiety comprises an antibody heavy chain domain.

30. The bispecific molecule of claim 29, wherein the antibody heavy chain domain of the glycan-binding moiety comprises a CH1 domain, a hinge region, a CH2 domain, a CH3 domain, or any combination thereof.

31. The bispecific molecule of claim 29 or claim 30, wherein the antibody heavy chain domain of the glycan-binding moiety comprises a CH2 domain, a CH3 domain, or both.

32. The bispecific molecule of any one of claims 29 to 31, wherein the antibody heavy chain domain of the glycan-binding moiety comprises a fragment crystallizable (Fc) region.

33. The bispecific molecule of claim 31 or claim 32, wherein the cell-targeting moiety comprises an antibody heavy chain comprising a CH3 domain, and wherein the bispecific molecule is a heterodimer comprising knobs-into-holes modified CH3 domains.

34. The bispecific molecule of any one of claims 1 to 32, wherein the bispecific molecule is a fusion protein comprising the cell-targeting moiety fused to the glycan-binding moiety.

35. The bispecific molecule of claim 34, wherein the cell-targeting moiety is fused directly to the glycan-binding moiety.

36. The bispecific molecule of claim 34, wherein the cell-targeting moiety is fused indirectly to the glycan-binding moiety via a linker.

37. The bispecific molecule of any one of claims 1 to 32, wherein the bispecific molecule is a conjugate comprising the cell-targeting moiety conjugated to the glycan-binding moiety.

38. A nucleic acid that encodes:
a cell-targeting moiety of the bispecific molecule of any one of claims 1 to 36;
a glycan-binding moiety of the bispecific molecule of any one of claims 1 to 36; or
both.

39. An expression vector comprising the nucleic acid of claim 38.

40. A pharmaceutical composition comprising:
the bispecific molecule of any one of claims 1 to 37; and
a pharmaceutically-acceptable carrier.

41. The pharmaceutical composition of claim 40, wherein the cell-targeting moiety is a cancer cell-targeting moiety.

42. The pharmaceutical composition of claim 40, wherein the cell-targeting moiety is an immune cell-targeting moiety.

43. A kit comprising:
one or more unit dosages of the pharmaceutical composition of any one of claims 40 to 42; and
instructions for administering the one or more unit dosages of the pharmaceutical composition to an individual in need thereof.

44. The kit of claim 43, comprising two or more unit dosages of the pharmaceutical composition.

45. The kit of claim 43 or claim 44, wherein the cell-targeting moiety is a cancer cell-targeting moiety, and wherein the instructions comprise instructions for administering the one or more unit dosages of the pharmaceutical composition to an individual in need of enhancement of anti-tumor immunity.

46. The kit of claim 43 or claim 44, wherein the cell-targeting moiety is an immune cell-targeting moiety, and wherein the instructions comprise instructions for administering the one or more unit dosages of the pharmaceutical composition to an individual in need of enhancement or suppression of an immune response.

47. A method of enhancing anti-tumor immunity in an individual in need thereof, comprising:
administering an effective amount of the pharmaceutical composition of claim 41 to the individual.

48. A method of enhancing or suppressing an immune response in an individual in need thereof, comprising:
administering an effective amount of the pharmaceutical composition of claim 42 to the individual.

49. The method according to claim 47 or claim 48, wherein the administering is by parenteral administration.

50. A bispecific molecule comprising:
a cell-targeting moiety fused to an Fc region; and
a moiety comprising a ligand-binding domain of a receptor fused to an Fc region,
wherein the cell-targeting moiety and the moiety comprising a ligand-binding domain of a receptor are heterodimerized via the Fc regions.

51. The bispecific molecule of claim 50, wherein the cell targeting moiety is as defined in any one of claims 2 to 5.

52. The bispecific molecule of claim 50 or claim 51, wherein the moiety comprising a ligand-binding domain of a receptor comprises the ligand-binding domain of a receptor that binds to a cell surface ligand.

53. The bispecific molecule of any one of claims 50 to 52, wherein the bispecific molecule is a heterodimer comprising knobs-into-holes modified CH3 domains.
